SEQUENCE LISTING (25 sequences)

SEQ ID NO: 1 - protein, 447 aa, RAT
MDMGNQHPSISRLQEIQREVKSIEQQVVGFSGLSDDKNYKRLERILTKQLFEIDSVDTEGKGDIQQARKRAAQDTERLLKELEQNANHPHRIEIKNIFQEAQALVKEKTVPFYSGSNCVTSEFEEAIQDIILRLTHVKTGGKISLRKARYHTLTKICAVQEIIEDCVRKQPSLPLSEDVHPSVAKINSVMCEVNKARGTLIALLMGVDSSETCRHLSCVLSGLMADLDALDVCGRTEIRNYRREVVEDINKLLKYLDLEEEADNTHAFDLGQNHSIIKIENVLKRMREMKNELLQAQSPPELYLRSKTELQGLIGQLDEVSLEKNPCIREARRRAVIEVQTLITYLDLKEALEKRKLFPCEETPPHKAVWNVLGNLSEIQGEVLSFGGNRTDKNYIRLEELLTKQLLTLDAVDPLGEEKCKAARKQAVKLAQNILSYLDMKSDEWEY

SEQ ID NO: 2 - protein, 447 aa, HUMAN
MDMGNQHPSISRLQEIQKEVKSVEQQVIGFSGLSDDKNYKKLERILTKQLFEIDSVDTEGKGDIQQARKRAAQETERLLKELEQNANHPHRIEIQNIFEEAQSLVREKIVPFYNGGNCVTDEFEEGIQDIILRLTHVKTGGKISLRKARYHTLTKICAVQEIIEDCMKKQPSLPLSEDAHPSVAKINFVMCEVNKARGVLIALLMGVNNNETCRHLSCVLSGLIADLDALDVCGRTEIRNYRREVVEDINKLLKYLDLEEEADTTKAFDLRQNHSILKIEKVLKRMREIKNELLQAQNPSELYLSSKTELQGLIGQLDEVSLEKNPCIREARRRAVIEVQTLITYIDLKEALEKRKLFACEEHPSHKAVWNVLGNLSEIQGEVLSFDGNRTDKNYIRLEELLTKQLLALDAVDPQGEEKCKAARKQAVRLAQNILSYLDLKSDEWEY

SEQ ID NO: 3 - protein, 447 aa, MOUSE
MDMGNQHPSISRLQEIQREVKAIEPQVVGFSGLSDDKNYKRLERILTKQLFEIDSVDTEGKGDIQQARKRAAQETERLLKELEQNANHPHRIEIQNIFKEAQALVKDKIVPFYSGGNCVTDEFEEGIQDIILRLTHVKTGGKVSLRKARYRTLTKICAVQEVIEDCMKKQPSLPLSEDVHPSVAKINSVMCEVNKARGTLIALLMGVDSSETCRHLSCVLSGLIADLDALDVCGRTEIRNYRREVVEDINKLLKYLDLEEEADSTHAFDLGQNHSIIKIENVLKRMREIKNELLQAQSPPELYLRAKTELQGLIGQLDEVSLEKNPCIREARRRAVIEVQILITYLDLKEALEKRKLFPCEEHPPHKAVWEILGNLSEILGEVLSFGGNRTDKNYIRLEELLTKQLLALDAVDPQGEEKCKAARKQAVKLAQNILSYLDMKSDEWEY

SEQ ID NO: 4 - DNA, 1709 bp, RAT
tgatgaaaattccctgagagggacgaccggctgcggggcccgcacggacgaggaggcgagttttctccgggctcgaagctgaggggcccgctgtgcgaggacggcgacaacgctgcggaagtggaggtggcggagcgcgatggcgcgtgcggcacagtgcttgtgaaactgaacacagaagtatggatatgggaaaccaacacccttctattagtaggcttcaggagatccaacgggaagtaaagagtatagagcagcaagtggttggcttcagtggcctgtcggatgacaagaattacaagagactggagaggattctgacgaaacaactttttgaaatcgactctgtggatacagaagggaaaggagacatccagcaagctaggaagagggcagcacaggacacagaacgccttctcaaagagttggagcagaacgcgaaccacccacaccggattgaaatcaagaatatctttcaagaagctcaggcccttgtgaaagaaaagactgtgcccttttacagtggaagcaactgtgtgactagcgagtttgaggaagccatccaggacatcattctgaggctgacacatgttaaaacaggagggaagatctccttgcggaaagcaaggtaccacacactaaccaagatctgtgcggtacaagagattattgaagactgtgtgaggaaacagccttccctgccactttccgaggatgtgcatccttctgtcgccaagatcaattctgtgatgtgtgaggtgaacaaggccagaggtactctgattgcacttctgatgggcgtggacagttctgagacttgcaggcacttatcatgcgtgctgtcaggactgatggctgatttagatgctctagatgtgtgtgggcgtacagagatcagaaattatcggagggaggtggtggaagatatcaacaaattactaaaatatctagatttagaagaggaagctgacaatacacatgcatttgacctgggacagaaccattccattataaagatagaaaatgtcctcaagaggatgagagaaatgaaaaatgaacttctccaggcacagagccctcctgaattgtacctgaggtccaagacagagctgcagggcttgatcgggcagctggacgaagtgagtctggagaaaaacccctgcatccgagaggccaggagaagagcggtgattgaggtgcagaccctcatcacgtacctggacctgaaggaggccctcgagaagaggaagctgttcccgtgtgaggagacccccccacataaggctgtgtggaatgtccttggaaacctgtcagagatccagggtgaagtcctctcctttggtgggaaccgaactgataagaactacattcgtctggaggagctactgaccaaacagttgctgactctggatgctgttgacccactgggagaagagaagtgtaaggctgccaggaagcaggccgtgaagctggcccagaacatcctcagctaccttgacatgaagtctgatgagtgggagtactgacggaccgctgatcctgccgctttgcactttctctcctctggagagctttccgctcatggagcctaaatgtaatgtctatgtgcatagtcaatctcagtatttatgatttaatcaaattatattcagtatttctgctgcttttgatgttgcaaaacgaatatcattatagcacattaccttttc

SEQ ID NO: 5 - DNA, 4285 bp, HUMAN
gaagacgcccggagcggctgctgcagccagtagcggccccttcaccggctgccccgctcagacctagtcgggaggggtgcgaggcatgcagctgggggcccagctccggtgccgcaccccgtaaagggctgatcttccacctcgccacctcagccacgggacgccaagaccgcatccaattcagacttcttttggtgcttgtgaaactgaacacaacaaaagtatggatatgggaaaccaacatccttctattagtaggcttcaggaaatccaaaaggaagtaaaaagtgtagaacagcaagttatcggcttcagtggtctgtcagatgacaagaattacaagaaactggagaggattctaacaaaacagctttttgaaatagactctgtagatactgaaggaaaaggagatattcagcaagctaggaagcgggcagcacaggagacagaacgtcttctcaaagagttggagcagaatgcaaaccacccacaccggattgaaatacagaacatttttgaggaagcccagtccctcgtgagagagaaaattgtgccattttataatggaggcaactgcgtaactgatgagtttgaagaaggcatccaagatatcattctgaggctgacacatgttaaaactggaggaaaaatctccttgcggaaagcaaggtatcacactttaaccaaaatctgtgcggtgcaagagataatcgaagactgcatgaaaaagcagccttccctgccgctttccgaggatgcacatccttccgttgccaaaatcaacttcgtgatgtgtgaggtgaacaaggcccgaggggtcctgattgcacttctgatgggtgtgaacaacaatgagacctgcaggcacttatcctgtgtgctctcggggctgatcgctgacctggatgctctagatgtgtgcggccggacagaaatcagaaattatcggagggaggtagtagaagatatcaacaaattattgaaatatctggatttggaagaggaagcagacacaactaaagcatttgacctgagacagaatcattccattttaaaaatagaaaaggtcctcaagagaatgagagaaataaaaaatgaacttctccaagcacaaaacccttctgaattgtacctgagctccaaaacagaattgcagggtttaattggacagttggatgaggtaagtcttgaaaaaaacccctgcatccgggaagccaggagaagagcagtgatcgaggtgcaaactctgatcacatatattgacttgaaggaggcccttgagaaaagaaagctgtttgcttgtgaggagcacccatcccataaagccgtctggaacgtccttggaaacttgtctgagatccagggagaagttctttcatttgatggaaatcgaaccgataagaactacatccggctggaagagctgctcaccaagcagctgctagccctggatgctgttgatccgcagggagaagagaagtgtaaggctgccaggaaacaagctgtgaggcttgcgcagaatattctcagctatctcgacctgaaatctgatgaatgggagtactgaaataccagagatctcacttttgatactgttttgcacttcatatgtgcttctatgtatagagagctttcagttcattgatttatacgtgcatatttcagtctcagtatttatgattgaagcaaattctattcagtatctgctgcttttgatgttgcaagacaaatatcattacagcacgttaacttttccattcggatcattatctgtatgatgtggtgtggtttgtttggtttgtccttttttttgcgtttttaatcagaaaacaaaatagaggcagcttttgtagattttaaatgggttgtgcaagcattaaaatgcaggtctttcagaatctagaactaggcataaccttacataatactaggaaaattatgagaaaggggaaatttttggttaaataagagtaaggttcaaacacaagcagtacatgttctgtttcattatgctcgatagaaggcttttttttcacttataaggcctgattggtcctacccagcttaacggggtggggtttttttgtttgttcagacagtctgttcttttgtaaacatttttagttggaaaaacagcatctgcattttccccatcctctacgttttagagaggaatcttgtttttgtgtgcaacataagaaaattatgaaaactaatagccaaaaaacctttgagattgcattaaagagaagggataaaggaccagcaataataccttgtaagttgcttttgtttgtaaaatctgagcttatagttttccttagtgagtaaattcataaggatgggaacatttaaattaagttaatgggcctttaaaaaaaaaaaaggaaacactcatacctgtagttggaggatgaatactggagacgggttaccaatgtcaggttatactaaaactaaatcagaaagtctgaatgtagcacataatggttctcttctgttgtccaaggctgtaaaatggacagccttgtcacacctccccggtgctgttttacaacgtgagggtagacgctgtcagtaacccagagggaccaggccttcctaggttttctaggcagtcagctgttaaccactcacttagtaaatgtcataactacacctgctccaggaccaatcagtgaaacctgctcggaattaaaggcttcctctgggtgcctgctgaacaactgagctcatgtcatgggcatgtggtggtttctctgttgcctgaaagagccattaaagtcagtcgtgcgtgaagcatctctcttctaaaggatgtgtatttccataaatgctttctgaggatccggtacaaaatgatttcccaaagttctgaagtgccttgagaacatgtgggtccgagtgttataacagactcctcccccgggtcaccttttgcctggtcatcctgttagagtacatctttggaaatccagggtaatattctctttcagagatgctcattgtgtaactctgtgtagggagatagtcactttaaacagctcaaagtagctagctaaaggagtagccttaaatacctaaaagatgacagaagcatagcccttaacaaatcttcagcttgtctctcagtatttcccaatcatgaaaatcccttgctatgtctttcctactagaaatgttctagaatcgctggacggtggggtcagagggcagtcggtatttaggccgtgagcttcccatactactgcaggtccaactcctggcaaccgcgggctcaaggcaggtcattggaatccacgttttggccacagtagttgtaggattgcttttctgtatcataattttagaatgctcttaaaatcttgaggaagagtttttattttttatttatttttgagatggagtctctgttgcccaggctgcagtgcagtggtgccatctcagctcactgcaacctccacctcccaggttcaagcgattctcctgcctcagccacctgagtagctgggagtacaggcatgtggcaccatgcctggctaatttttgtatttttaatagagttgagatttcaccatgatggtcaggctggtctcgaactcctgacctcgtgatccgcccgcctcggccccccaaagtgctgggattaacgggtgtgagccacggcgcccagcccaggaagagtttttaaattagagctctgtttaattataccactgggaaatcatggttacgcttcaggcatattcttccccagagtactacttacattttaaatttcattttgtaaagttaaatgtcagcattccctttaaaagtgtccattgttctttgaaagtagacgtttcagtcattcttttcaaacaagtgtttgtgtaccttttgccaagctgtgggcatcgtgtgtgagtacagggtgctcagctcttccaccgtcattttgaattgttcacatgggtaattggtcatggaaatgatcagattgaccttgattgactgtcaggcatggctttgtttctagtttcaatctgttctcgttccttgtaccggattattctactcctgcaatgaaccctgttgacaccggatttagctcttgtcggccttcgtggggagctgtttgtgttaatatgagctactgcatgtaattcttaaactgggcttgtcacattgtattgtatttttgtgatctgtaatgaaaagaatctgtactgcaagtaaaacctactccccaaaaatgtgtggctttgggtctgcattaaacgctgtagtccatgttcatgcc

SEQ ID NO: 6 - DNA, 93 bp, Artificial Sequence
  Synthetic Primer
aaaaaaatcaagataactaagaatattctgcgcaacaagcttcttgcgcagaatattctcagctatctcgacggtgtttcgtcctttccacaa

SEQ ID NO: 7 - DNA, 2136 bp, MOUSE
tggaaaggttggcgttacggcggcgtcgcaaagcccgagccgacgcgcggcgaggccgaccgtgcgcattccctgggacggacgccgggcggcggggcccgcgcgggacgaggagaggacaagttttccccgggctcactgctgacgggcccgctctgcgaggacggcgacaacgctgcggaagtgtaggtggcggggcgcgatggcgcgtgcggcccagtgcttgtgaaactgaacacagaagtatggatatgggaaaccaacacccctccattagtaggcttcaggagatccagcgggaagtaaaggctatagagccgcaagtggtcggcttcagcggcctgtcggatgacaagaactacaagaggctggagaggattctgaccaaacaactttttgaaattgactctgtggatacggaaggaaaaggagatatccagcaagctaggaagcgggcggcacaggagacagaacgtctcctcaaggagttggagcagaatgccaaccacccacaccgcatcgaaatccagaatatctttaaggaggctcaggcccttgtgaaagacaagattgtgcccttttacagcggaggcaactgtgtgactgacgagtttgaggaaggcatccaggacatcattctgaggctgacgcacgtgaagaccggagggaaggtctccttgcggaaagccaggtaccgcacgctcaccaagatctgcgcggtacaggaggttattgaggactgtatgaagaagcagccgtccctgccactgtctgaggatgtgcatccttcagtggccaaaatcaactctgtcatgtgcgaggtgaacaaggccagagggacgctgatcgcacttctgatgggcgtggacagttccgagacttgcaggcacttatcctgcgtgctgtcaggactgattgctgacttagatgctctagatgtgtgtgggcgtacagagatcagaaattatcggagggaggtggtggaagatatcaacaaattactgaaatacctagacttagaagaggaagctgacagtacacatgcatttgacctgggacagaaccattccattataaaaatagaaaatgttctcaagaggatgagagagataaaaaatgaacttctccaggcgcagagccctcctgagttgtacctgagggccaagacagagctgcagggcttgatcggacagctggatgaagtgagtctggagaaaaacccctgcatccgggaggccaggaggagggcggtgattgaggtgcagatcctgatcacgtacctggacctgaaggaagccctcgagaagaggaagctgttcccatgtgaggagcaccccccacacaaggccgtgtgggagatccttgggaacctgtctgagatcctgggcgaagtcctttcatttggtggaaaccgaactgataagaactacatccgtctggaggagctactgaccaaacagttgctggctctggatgccgttgacccacagggagaagagaagtgtaaggctgccaggaaacaggctgtgaaactggcccagaacatcctcagctacctggacatgaagtcgacgagtgggagtattgatgggccaggagctgatctcgctggttcgcacctcatctcctttagagagctctcagctcatggagcctaaatgtaatttctatgtgcatagtcaatctcagtatttatgatttaatcaaattatattcagtatttctgctgcttttgatgttgcaaacgaatatcattatagcacattaccttttccatctggctcattatccttacaatgtgtggtttgtgtttattttgtgtgtttttaatgggaacacgaaaaaggcagcattcctaagttttaaatgggttgtgcaagcattagatgcagaatttgagagtctagaaacagattagacttttctaactctagcctaatttttaactctagcctaattaagaggaggaggagggaattccagggttgaataaaatccaagcatagaaagagacgctttattatgctaaacagtgctgggactttttaaagttacaaggcctaatgggtcctgcccagttttaatggggtggggtttttgtttggatggtctgttttatcataaacatttttgctt

SEQ ID NO: 8 - protein, 447 aa, HUMAN
MDMGNQHPSISRLQEIQKEVKSVEQQVIGFSGLSDDKNYKKLERILTKQLFEIASVDTEGKGDIQQAAKRAAQETERLLKELEQNANHPHRIEIQNIFEEAQSLVREKIVPFYNGGNCVTDEFEEGIQDIILRLTHVKTGGKISLRKARYHTLTKICAVQEIIEDCMKKQPSLPLSEDAHPSVAKINFVMCEVNKARGVLIALLMGVNNNETCRHLSCVLSGLIADLAALDVCGRTEIRNYAREVVEDINKLLKYLDLEEEADTTKAFDLRQNHSILKIEKVLKRMREIKNELLQAQNPSELYLSSKTELQGLIGQLAEVSLEKNPCIREAARRAVIEVQTLITYIDLKEALEKRKLFACEEHPSHKAVWNVLGNLSEIQGEVLSFDGNRTDKNYIRLEELLTKQLLALAAVDPQGEEKCKAAAKQAVRLAQNILSYLDLKSDEWEY

SEQ ID NO: 9 - DNA, 1344 bp, Human
atggatatgggaaaccaacatccttctattagtaggcttcaggaaatccaaaaggaagtaaaaagtgtagaacagcaagttatcggcttcagtggtctgtcagatgacaagaattacaagaaactggagaggattctaacaaaacagctttttgaaatagcctctgtagatactgaaggaaaaggagatattcagcaagctgcgaagcgggcagcacaggagacagaacgtcttctcaaagagttggagcagaatgcaaaccacccacaccggattgaaatacagaacatttttgaggaagcccagtccctcgtgagagagaaaattgtgccattttataatggaggcaactgcgtaactgatgagtttgaagaaggcatccaagatatcattctgaggctgacacatgttaaaactggaggaaaaatctccttgcggaaagcaaggtatcacactttaaccaaaatctgtgcggtgcaagagataatcgaagactgcatgaaaaagcagccttccctgccgctttccgaggatgcacatccttccgttgccaaaatcaacttcgtgatgtgtgaggtgaacaaggcccgaggggtcctgattgcacttctgatgggtgtgaacaacaatgagacctgcaggcacttatcctgtgtgctctcggggctgatcgctgacctggctgctctagatgtgtgcggccggacagaaatcagaaattatgcgagggaggtagtagaagatatcaacaaattattgaaatatctggatttggaagaggaagcagacacaactaaagcatttgacctgagacagaatcattccattttaaaaatagaaaaggtcctcaagagaatgagagaaataaaaaatgaacttctccaagcacaaaacccttctgaattgtacctgagctccaaaacagaattgcagggtttaattggacagttggctgaggtaagtcttgaaaaaaacccctgcatccgggaagccgcgagaagagcagtgatcgaggtgcaaactctgatcacatatattgacttgaaggaggcccttgagaaaagaaagctgtttgcttgtgaggagcacccatcccataaagccgtctggaacgtccttggaaacttgtctgagatccagggagaagttctttcatttgatggaaatcgaaccgataagaactacatccggctggaagagctgctcaccaagcagctgctagccctggctgctgttgatccgcagggagaagagaagtgtaaggctgccgcgaaacaagctgtgaggcttgcgcagaatattctcagctatctcgacctgaaatctgatgaatgggagtactga

SEQ ID NO: 10 - DNA, 1291 bp, HUMAN
acgccgcgctcagcttccatcgctgggcggtcaacaagtgcgggcctggctcagcgcgggggggcgcggagaccgcgaggcgaccgggagcggctgggttcccggctgcgcgcccttcggccaggccgggagccgcgccagtcggagcccccggcccagcgtggtccgcctccctctcggcgtccacctgcccggagtactgccagcgggcatgaccgacccaccaggggcgccgccgccggcgctcgcaggccgcggatgaagaagaaaacccggcgccgctcgacccggagcgaggagttgacccggagcgaggagttgaccctgagtgaggaagcgacctggagtgaagaggcgacccagagtgaggaggcgacccagggcgaagagatgaatcggagccaggaggtgacccgggacgaggagtcgacccggagcgaggaggtgaccagggaggaaatggcggcagctgggctcaccgtgactgtcacccacagcaatgagaagcacgaccttcatgttacctcccagcagggcagcagtgaaccagttgtccaagacctggcccaggttgttgaagaggtcataggggttccacagtcttttcagaaactcatatttaagggaaaatctctgaaggaaatggaaacaccgttgtcagcacttggaatacaagatggttgccgggtcatgttaattgggaaaaagaacagtccacaggaagaggttgaactaaagaagttgaaacatttggagaagtctgtggagaagatagctgaccagctggaagagttgaataaagagcttactggaatccagcagggttttctgcccaaggatttgcaagctgaagctctctgcaaacttgataggagagtaaaagccacaatagagcagtttatgaagatcttggaggagattgacacactgatcctgccagaaaatttcaaagacagtagattgaaaaggaaaggcttggtaaaaaaggttcaggcattcctagccgagtgtgacacagtggagcagaacatctgccaggagactgagcggctgcagtctacaaactttgccctggccgagtgaggtgtagcagaaaaaggctgtgctgccctgaagaatggcgccaccagctctgccgtctctggatcggaatttacctgatttcttcagggctgctgggggcaactggccatttgccaattttcctactctcacactggttctcaatgaaaaatagtgtctttgtgatttgagtaaagctcctattctgtttttcacaaaaaaaaaaaaa

SEQ ID NO: 11 - protein, 345 aa, HUMAN
MAQRGGARRPRGDRERLGSRLRALRPGREPRQSEPPAQRGPPPSRRPPARSTASGHDRPTRGAAAGARRPRMKKKTRRRSTRSEELTRSEELTLSEEATWSEEATQSEEATQGEEMNRSQEVTRDEESTRSEEVTREEMAAAGLTVTVTHSNEKHDLHVTSQQGSSEPVVQDLAQVVEEVIGVPQSFQKLIFKGKSLKEMETPLSALGIQDGCRVMLIGKKNSPQEEVELKKLKHLEKSVEKIADQLEELNKELTGIQQGFLPKDLQAEALCKLDRRVKATIEQFMKILEEIDTLILPENFKDSRLKRKGLVKKVQAFLAECDTVEQNICQETERLQSTNFALAE

SEQ ID NO: 12 - DNA, 1311 bp, Human
tagtcgggcggggttgtgagacgccgcgctcagcttccatcgctgggcggtcaacaagtgcgggcctggctcagcgcgggggggcgcggagaccgcgaggcgaccgggagcggctgggttcccggctgcgcgcccttcggccaggccgggagccgcgccagtcggagcccccggcccagcgtggtccgcctccctctcggcgtccacctgcccggagtactgccagcgggcatgaccgacccaccaggggcgccgccgccggcgctcgcaggccgcggatgaagaagaaaacccggcgccgctcgacccggagcgaggagttgacccggagcgaggagttgaccctgagtgaggaagcgacctggagtgaagaggcgacccagagtgaggaggcgacccagggcgaagagatgaatcggagccaggaggtgacccgggacgaggagtcgacccggagcgaggaggtgaccagggaggaaatggcggcagctgggctcaccgtgactgtcacccacagcaatgagaagcacgaccttcatgttacctcccagcagggcagcagtgaaccagttgtccaagacctggcccaggttgttgaagaggtcataggggttccacagtcttttcagaaactcatatttaagggaaaatctctgaaggaaatggaaacaccgttgtcagcacttggaatacaagatggttgccgggtcatgttaattgggaaaaagaacagtccacaggaagaggttgaactaaagaagttgaaacatttggagaagtctgtggagaagatagctgaccagctggaagagttgaataaagagcttactggaatccagcagggttttctgcccaaggatttgcaagctgaagctctctgcaaacttgataggagagtaaaagccacaatagagcagtttatgaagatcttggaggagattgacacactgatcctgccagaaaatttcaaagacagtagattgaaaaggaaaggcttggtaaaaaaggttcaggcattcctagccgagtgtgacacagtggagcagaacatctgccaggagactgagcggctgcagtctacaaactttgccctggccgagtgaggtgtagcagaaaaaggctgtgctgccctgaagaatggcgccaccagctctgccgtctctggatcggaatttacctgatttcttcagggctgctgggggcaactggccatttgccaattttcctactctcacactggttctcaatgaaaaatagtgtctttgtgatttgagtaaagctcctattctgtttttcacaaaaaaaaaaaaa

SEQ ID NO: 13 - protein, 345 aa, Human; identical to SEQ ID NO: 11

SEQ ID NO: 14 - DNA, 1556 bp, HUMAN
gcagccgcggtgtcgcgaagtcctcccgggttgcccccgcggcgtcagagggagggcgggcgccgcgttggtgacggcgaccctgcagcccaaggagcgctccactcgctgccgccggagggccggtgacctcttggctaccccgcgtcggaggcttagatggctcaggcgaagatcaacgctaaagccaacgaggggcgcttctgccgctcctcctccatggctgaccgctccagccgcctgctggagagcctggaccagctggagctcagggttgaagctttgagagaagcagcaactgctgttgagcaagagaaagaaatccttctggaaatgatccacagtatccaaaatagccaggacatgaggcagatcagtgacggagaaagagaagaattaaatctgactgcaaaccgtttgatgggaagaactctcaccgttgaagtgtcagtagaaacaattagaaacccccagcagcaagaatccctaaagcatgccacaaggattattgatgaggtggtcaataagtttctggatgatttgggaaatgccaagagtcatttaatgtcgctctacagtgcatgttcatctgaggtgccacatgggccagttgatcagaagtttcaatccatagtaattggctgtgctcttgaagatcagaagaaaattaagagaagattagagactctgcttagaaatattgaaaactctgacaaggccatcaagctattagagcattctaaaggagctggttccaaaactctgcaacaaaatgctgaaagcagattcaattagtcttcaaacctaagagcatttacacaatacacaaggtgtaaaaatgataaaatactattttaattgataactagttctttgttaggtataaccacttagttgacactgatagttgtttcagatgaggaaaatattccatcaagtatcttcagttttgtgaataacaaaactagcaatattttaattatctatctagagattttttagattgaattcttgtcttgtactaggatctagcatatttcactattctgtggatgaatacatagtttgtggggaaaacaaacgttcagctaggggcaaaaagcatgactgctttttcctgtctggcatggaatcacgcagtcaccttgggcatttagtttactagaaattctttactgaagcagcacacacatttactacacacacagtgttaacaaagcactgtgcttagagggtaaaaaggaatcacaaaacaagaatctttccaaagttgtctcattcagcaatgttaaggcatctgtatcaaattattttggatgtaaagattcctgtgtctcataatatgaatgtattttttgatatacaagaaactgacataaaatgtgagaaaaccacctataatttaccactgtgaacaattatatatctatctgcttcatcttttctcaaatgcatcaattctctaaaattcctatattgtaacttgcctttttttaaaaaagttagatgctgatataaagtctgcttaattgtcaacttaaaaaaaaaaaaaaaaa

SEQ ID NO: 15 - protein, 211 aa, HUMAN
MAQAKINAKANEGRFCRSSSMADRSSRLLESLDQLELRVEALREAATAVEQEKEILLEMIHSIQNSQDMRQISDGEREELNLTANRLMGRTLTVEVSVETIRNPQQQESLKHATRIIDEVVNKFLDDLGNAKSHLMSLYSACSSEVPHGPVDQKFQSIVIGCALEDQKKIKRRLETLLRNIENSDKAIKLLEHSKGAGSKTLQQNAESRFN

SEQ ID NO: 16 - DNA, 2605 bp, HUMAN
gcggagctccgcatccaaccccgggccgcggccaacttctctggactggaccagaagtttctagccggccagttgctacctccctttatctcctccttcccctctggcagcgaggaggctatttccagacacttccacccctctctggccacgtcacccccgcctttaattcataaaggtgcccggcgccggcttcccggacacgtcggcggcggagaggggcccacggcggcggcccggccagagactcggcgcccggagccagcgccccgcacccgcgccccagcgggcagaccccaacccagcatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtgcaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtagcctctgccctgtaaaaatcagactcggaaccgatgtgtgctttagggaattttaagttgcatgcatttcagagactttaagtcagttggtttttattagctgcttggtatgcagtaacttgggtggaggcaaaacactaataaaagggctaaaaaggaaaatgatgcttttcttctatattcttactctgtacaaataaagaagttgcttgttgtttgagaagtttaaccccgttgcttgttctgcagccctgtctacttgggcacccccaccacctgttagctgtggttgtgcactgtcttttgtagctctggactggaggggtagatggggagtcaattacccatcacataaatatgaaacatttatcagaaatgttgccattttaatgagatgattttcttcatctcataattaaaatacctgactttagagagagtaaaatgtgccaggagccataggaatatctgtatgttggatgactttaatgctacattttaaaaaaagaaaataaagtaataatataactcaaaaaaaaaaaaaaaaaaaaaaaaaaaaaaaaaaaaaaaaa

SEQ ID NO: 17 - protein, 575 aa, HUMAN
MSAATHSPMMQVASGNGDRDPLPPGWEIKIDPQTGWPFFVDHNSRTTTWNDPRVPSEGPKETPSSANGPSREGSRLPPAREGHPVYPQLRPGYIPIPVLHEGAENRQVHPFHVYPQPGMQRFRTEAAAAAPQRSQSPLRGMPETTQPDKQCGQVAAAAAAQPPASHGPERSQSPAASDCSSSSSSASLPSSGRSSLGSHQLPRGYISIPVIHEQNVTRPAAQPSFHQAQKTHYPAQQGEYQTHQPVYHKIQGDDWEPRPLRAASPFRSSVQGASSREGSPARSSTPLHSPSPIRVHTVVDRPQQPMTHRETAPVSQPENKPESKPGPVGPELPPGHIPIQVIRKEVDSKPVSQKPPPPSEKVEVKVPPAPVPCPPPSPGPSAVPSSPKSVATEERAAPSTAPAEATPPKPGEAEAPPKHPGVLKVEAILEKVQGLEQAVDNFEGKKTDKKYLMIEEYLTKELLALDSVDPEGRADVRQARRDGVRKVQTILEKLEQKAIDVPGQVQVYELQPSNLEADQPLQAIMEMGAVAADKGKKNAGNAEDPHTETQQPEATAAATSNPSSMTDTPGNPAAP

SEQ ID NO: 18 - DNA, 2182 bp, HUMAN
aggtaagaggaaactccattggataaatggcgaggaaacgtatactccctcttaaggaacacggtgtcttccttcgtctccgggttcccgagaccccagagtcactgacctccgtccctcagctttcggggttcggcagcagaaggggcgggcccgggcctgggattggctggcgtcgtccgacccccttcgctgctctccattcgcaatcgcccgcgggcgcctgcgcgatgggtcggccgtggggagcggggcgggaagcgcttcagggcagcggatcccatgtcggccctgaggcgctcgggctacggccccagtgacggtccgtcctacggccgctactacgggcctgggggtggagatgtgccggtacacccacctccacccttatatcctcttcgccctgaacctccccagcctcccatttcctggcgggtgcgcgggggcggcccggcggagaccacctggctgggagaaggcggaggaggcgatggctactatccctcgggaggcgcctggccagagcctggtcgagccggaggaagccaccaggagcagccaccatatcctagctacaattctaactattggaattctactgcgagatctagggctccttacccaagtacatatcctgtaagaccagaattgcaaggccagagtttgaattcttatacaaatggagcgtatggtccaacataccccccaggccctggggcaaatactgcctcatactcaggggcttattatgcacctggttatactcagaccagttactccacagaagttccaagtacttaccgttcatctggcaacagcccaactccagtctctcgttggatctatccccagcaggactgtcagactgaagcaccccctcttagggggcaggttccaggatatccgccttcacagaaccctggaatgaccctgccccattatccttatggagatggtaatcgtagtgttccacaatcaggaccgactgtacgaccacaagaagatgcgtgggcttctcctggtgcttatggaatgggtggccgttatccctggccttcatcagcgccctcagcaccacccggcaatctctacatgactgaaagtacttcaccatggcctagcagtggctctccccagtcacccccttcacccccagtccagcagcccaaggattcttcatacccctatagccaatcagatcaaagcatgaaccggcacaactttccttgcagtgtccatcagtacgaatcctcggggacagtgaacaatgatgattcagatcttttggattcccaagtccagtatagtgctgagcctcagctgtatggtaatgccaccagtgaccatcccaacaatcaagatcaaagtagcagtcttcctgaagaatgtgtaccttcagatgaaagtactcctccgagtattaaaaaaatcatacatgtgctggagaaggtccagtatcttgaacaagaagtagaagaatttgtaggaaaaaagacagacaaagcatactggcttctggaagaaatgctaaccaaggaacttttggaactggattcagttgaaactgggggccaggactctgtacggcaggccagaaaagaggctgtttgtaagattcaggccatactggaaaaattagaaaaaaaaggattatgaaaggatttagaacaaagtggaagcctgttactaacttgaccaaagaacacttgatttggttaattaccctctttttgaaatgcctgttgatgacaagaagcaatacattccagctttcctttgattttatacttgaaaaactggcaaaggaatggaagaatattttagtcatgagttgttttcagttttcagacgaatgaatgtaataggaaactatggagttaccaatattgccaagtagactcactccttaaaaaatttatggatatctacaagctgcttcttaccagcaggagggaaacacacttcacacaacaggcttatcagaaacctaccagatgaaactggatataatctgagacaaacaggatgtgtttttttaaacatctggatatcttgtcacatttttgtacattgtgactgctttcaacatatacttcatgtgtaattatagcttagactttagccttcttggacttctgttttgttttgttatttgcagtttacaaatatagtattattctct

SEQ ID NO: 19 - protein, 457 aa, HUMAN
MSALRRSGYGPSDGPSYGRYYGPGGGDVPVHPPPPLYPLRPEPPQPPISWRVRGGGPAETTWLGEGGGGDGYYPSGGAWPEPGRAGGSHQEQPPYPSYNSNYWNSTARSRAPYPSTYPVRPELQGQSLNSYTNGAYGPTYPPGPGANTASYSGAYYAPGYTQTSYSTEVPSTYRSSGNSPTPVSRWIYPQQDCQTEAPPLRGQVPGYPPSQNPGMTLPHYPYGDGNRSVPQSGPTVRPQEDAWASPGAYGMGGRYPWPSSAPSAPPGNLYMTESTSPWPSSGSPQSPPSPPVQQPKDSSYPYSQSDQSMNRHNFPCSVHQYESSGTVNNDDSDLLDSQVQYSAEPQLYGNATSDHPNNQDQSSSLPEECVPSDESTPPSIKKIIHVLEKVQYLEQEVEEFVGKKTDKAYWLLEEMLTKELLELDSVETGGQDSVRQARKEAVCKIQAILEKLEKKGL

SEQ ID NO: 20 - DNA, 3853 bp, Xenopus laevis
ggcacgaggcgagactgtggtgcaattcccgtagcgctggcctgccggggagcaggagctgaaaagctggacatttaaacagcgattcggggcgagttgtgttatgaaaatcgggactgttgatcggttgaagaatggcagctaatgagaaaatggaagtgacggtgaaaacactggattcgcaaacaagaacattcacagtggagacagagatctcagtaaaggatttcaaagcacatatttcatctgatgttgggatttcgccagaaaagcaacggcttatatatcaggggcgagtcctgcaagaggacaaaaaactgaaagaatacaatgttgatgggaaggtcattcacttggttgagcgagcgcctcctcagacacaaccttcaacaggtggaccatctacgtcttcaagtacttcacccaccagcagtaatgcagcacctgtacctggggctcctgaaaggaatggaaacagctatgttatggttgggacattcaaccttccgcatgtaatgtctggtctggtgaggcagcagaggccaagtgtctctacagtgaatgggaatgatggctccacacttgatgttcatataaatctggatcaacagctccctgtccagagtgaaccacgggttcgtcttgttttggctcagcacatactgcaggacatacagaggatactagatcgtttggagggacaagcagttaatgagcaggctgcggaaccaatggacacagcagagtctgaaggagaggccagcagtagagaaacgttgcctcagactacacagaatacagatgggcagtcgaatacaacacctaccagtcacccttcaccatcagaatatgttgaggttctacaatctctgtcccgggttgaggaacgtctagcccctttcatgcaacgttacagggaaatcctcagtagtgctacctctgatgcctatgagaatcaggaagaaagggagcagtcccaacggataattaatttggttggagaatctcttcgtctcttggggaatgccctcgttgctgtatccgacttgcgctgcaatctctcgtctgcttctcccagacacttgcatgttgttcgcccaatgtcccattactctgggcccatgttactgcagcaggctgctataccaatacagattaatgttggtaccacagtcactgcaactgggaatgggacccatgctggtcacatgccttcagatgggaatgctgctcaacctccttcaaccaatacttcagaaccacagcgacctaacactgagaaccaacctccatccaatggagagagacctgcttctgatgcccctcccacttcagtcccacatccccaccctcgtgtgatacgcattacacaccagactgtagagcctgtcatgatgatgcatatgaacattcaggattctgcatctggtggacctacaaccattcccccaccaactgcaggacatgggggaagtgcacacatacatatgccagggttacctcctgagttcatgcaggcaatttcacaccagataacacagcaagctatggcagcagcttcagggcaacaaattcctggcttccaagcaccaccacgctttgtttttactagacctgctgccccttcatttcaattccagcctgggacagctactaccccaccaggaccaggaggggcaactaccactgtaccaggagcaacggtgggcccagcaggaaatgcctccttggctcagatgatcagtgggctagttggacagcttctgatgcacccagttgttgttgctcagggtggcagcagcacttcctcttctacttcctcttcaacattcacatcgacgtcctcctctgcctcctcttcatcctccactgataccacttcaacaacaaccacctcttctacagccaaccccactgtgtcctcagttccctcttcccagcccccacctggcacagatcaacacctctctcaacttctgggctctttattgggaactgccagctcagggatgtcaaacatcactatgggttcgccaagcattacagtgacagtgcctggaatgccggcctttttacagggcgtcacagatatattacaggctacacagactgttcctgtttccaccgctcctacccagtcggcttctcaggctcctcctccttcatcacctccaccacctcctgcccattcttcaccacctccggctgccgcaccagagtctcttcctccagagtttttcacaagtgttgttcagggtgtcctttcgtccatgctggggtctttgagtgcagctgatcagagtggaacagaaagcattgcagctttcattcagaggctcagtggcacccacaacatcttccagcctgatgcagaggggcctggaggattttttggtgacttactcaccttgatttgtcacaatttctcgctggtggatatggtaatgttactgcatggtcattcccagcctttgcagaacctgcagcctcaactgcgatccttctttcttcaggagtatctacaccaggtggatcccacccctaataacatccagatggcatcaagaaatcttaccaatggcctggaagagtacatacgagaaagctttgcctccgttactgtccgtgatgatgttgatatcacaagaacaaacattgaatttctacaggatcagttcaacaggatcacaacccacatcctgcattgtgcagattccacatttggacaacggcttctcgagatgtgtaaccagtcactttttgaatggcttgctctcaatctgtattgtctcagaggggaccagaatgctctgacatctgtcattaatgagagaattcgaaggctctctcttgatgtttcacctgtgctcgtctcgtgggtgacatctgtcctatccctccgtcttcaagtcctgttgggacagatgcctgtgactgaaggcgagatacagagacatgtacggagagtgggagatgctcctcaggttcctgaaccatcatctcaggagcaacctatggaaactatgcctgtcgattgccagaatggtgctgcctctccagtccttgccacccacagtggaggagttcttttccttccccctcaaagttccgtacccaccatatgcccagattctgatcacccaacacaagaagacggcggatctgaacaatgggcagcatctgtgcccccagaatgggttcctgtcatcagacaagatatgcagaaccaacgcaagataaaacagcagccacccttgagtgatgcctacctgagtggcatgcctgcaaagcgccgcaaaacaatgcaaggcgaggggccacacctttccctctctgaagctgtgagccgtgcaatgaaagccacaggagctaaacctgagagcagcgcagaatgtgtaaggagggagctggataacagtgaggcacaaggaaggtacagagaacagctgtgtcaggatatccagaaaacccttcaagacaacgagtcctacagtgcccagagattccccaacacacagagggcattcaggggggacccctaaataatcactcaaactctccttcagttatgtatatacactgcagaaagttcttttctgatcacatcccagtacactctgcacacggacgttgcatttaatttctgtaatatgtgcacaacaaagccttacagttgttacccctgcactccccacgcataatctaataaacctgagtctaaaataggggatccaattgccgtagatgaagcccaggcaagagtacatttctgctgtcaatcttccccagtctttgttgtgtgttctgtgagatttgtccattttgcaatataaataaaagaaccctgcccatt

SEQ ID NO: 21 - protein, 1135 aa, Xenopus laevis
MAANEKMEVTVKTLDSQTRTFTVETEISVKDFKAHISSDVGISPEKQRLIYQGRVLQEDKKLKEYNVDGKVIHLVERAPPQTQPSTGGPSTSSSTSPTSSNAAPVPGAPERNGNSYVMVGTFNLPHVMSGLVRQQRPSVSTVNGNDGSTLDVHINLDQQLPVQSEPRVRLVLAQHILQDIQRILDRLEGQAVNEQAAEPMDTAESEGEASSRETLPQTTQNTDGQSNTTPTSHPSPSEYVEVLQSLSRVEERLAPFMQRYREILSSATSDAYENQEEREQSQRIINLVGESLRLLGNALVAVSDLRCNLSSASPRHLHVVRPMSHYSGPMLLQQAAIPIQINVGTTVTATGNGTHAGHMPSDGNAAQPPSTNTSEPQRPNTENQPPSNGERPASDAPPTSVPHPHPRVIRITHQTVEPVMMMHMNIQDSASGGPTTIPPPTAGHGGSAHIHMPGLPPEFMQAISHQITQQAMAAASGQQIPGFQAPPRFVFTRPAAPSFQFQPGTATTPPGPGGATTTVPGATVGPAGNASLAQMISGLVGQLLMHPVVVAQGGSSTSSSTSSSTFTSTSSSASSSSSTDTTSTTTTSSTANPTVSSVPSSQPPPGTDQHLSQLLGSLLGTASSGMSNITMGSPSITVTVPGMPAFLQGVTDILQATQTVPVSTAPTQSASQAPPPSSPPPPPAHSSPPPAAAPESLPPEFFTSVVQGVLSSMLGSLSAADQSGTESIAAFIQRLSGTHNIFQPDAEGPGGFFGDLLTLICHNFSLVDMVMLLHGHSQPLQNLQPQLRSFFLQEYLHQVDPTPNNIQMASRNLTNGLEEYIRESFASVTVRDDVDITRTNIEFLQDQFNRITTHILHCADSTFGQRLLEMCNQSLFEWLALNLYCLRGDQNALTSVINERIRRLSLDVSPVLVSWVTSVLSLRLQVLLGQMPVTEGEIQRHVRRVGDAPQVPEPSSQEQPMETMPVDCQNGAASPVLATHSGGVLFLPPQSSVPTICPDSDHPTQEDGGSEQWAASVPPEWVPVIRQDMQNQRKIKQQPPLSDAYLSGMPAKRRKTMQGEGPHLSLSEAVSRAMKATGAKPESSAECVRRELDNSEAQGRYREQLCQDIQKTLQDNESYSAQRFPNTQRAFRGDP

SEQ ID NO: 22 - DNA, 3708 bp, MOUSE
gcggcggcggctcctccgggtggtcggctccctcccatctcggccggcccgggcccgtctcgccccccagaaactcactgttcggggctgtggactttctcatcgtgccccacgaaagtaaatcgggggagactgggggagccggaagtaccgcctcgagatccccccaaatacgatcggggaaacggaagtggcccttggtgccaggttgggggagaccggaagtgacgagagctgtcggccatggagccgagtgatagtgccagtaccgctatggaggagcctgacagcctggaggtactggtgaagaccctggactctcagactcggacttttattgtgggggcccagatgaatgtaaaggagtttaaggaacacatagctgcctctgtcagcatcccttccgagaaacagcggctcatctaccagggccgggtcctacaagacgacaagaagctccaggagtacaacgttgggggaaaggttattcacctggtggaacgggctcctcctcagactcagcttccttctggagcatcttctgggacaggctctgcctcagcaactcatggtggggcacccctgcctggcactcggggccctggggcttctgttcatgaccggaatgccaacagctatgtcatggttggaaccttcaatcttcctagtgacggctctgctgtggatgttcacatcaacatggaacaggccccaattcagagtgagccccgggtccggctggtgatggctcagcacatgattagggatatacagaccttactgtcccggatggagtgtcgagggggaacccaagctcaggccagtcagccacccccgcagacaccgcagactgtggcctcagagacggtagccttgaactcacaaacatcagaaccagtcgaaagtgaagctcctcctcgagagcccatggagtcagaagaaatggaggaacgtcccccaacccagactccagagcttgcgccctcaggcccagctcccgcaggcccagctcccgcaggcccagctcccgcgccagagacaaatgcacccaaccacccttcccccgcagagcacgtggaggtgctccaggagcttcagcgcttgcagcgccgtcttcagcctttcctacagcgctactgcgaggtcctgggcgctgctgccaccacagactacaacaacaaccatgagggccgtgaggaggaccagaggttgatcaacttggttggggagagcctgcggctactgggcaacactttcgtggcattgtctgatctgcgctgcaatctagcctgtgcacccccacggcacctgcacgtggttcggcctatgtctcactacacgactcccatggtgctccagcaggcagccattcccattcagatcaatgtggggactactgtgaccatgacaggcaatggggctcggcctccaccagctcctggtgcggaggcagcaaccccaggttctgcccaggccacatccctgcctccctcttccaccactgttgattcatcaactgaaggagctcccccaccagggccagcaccgccaccagcctccagccacccacgggtcatccggatttcccaccagagtgtggagcccgtggtcatgatgcacatgaacattcaagattctggagcacagcctggtggtgtgccaagtgctcccactggtccactgggacctcctggtcatggacagaccctgggacagcaggtgcccggcttcccaacagcaccaactcgggtggtgattgcccggccaactcctccacaggctcggccttcccatcctgggggacctccagtctctggagctctgcagggcgctgggctgggtacaaacacttcattggcccagatggtgagcggccttgtggggcaacttcttatgcagcctgttcttgtggctcaggggactccaggaatggctcaggctcaggcccaggcccaggctcaggcccaggcccaggcccaggctccagctccggctccggctccagctccagctcctgccactgcttcagctagtgctggtactaccaacacagctaccacagctggccctgctcctgggggtcctgcccagcctccacctcctcagccctctgcagctgatcttcagttctctcagctcctgggaaatctgctggggcctgcagggccaggggctggggggccaggcatggcttctcccaccatcactgtggcaatgcctggtgtccctgcttttctccagggcatgactgattttttgcaggcatcacagactgcccctccaccccctccacctcctccacccccaccccctgccccagagcagcagagcacacccccaccagggtctccttctggtggaacagcaagccctggaggcttaggtcctgagagcctgccaccagagtttttcacctcagtggtgcagggcgtgttgagctccctcctgggctccttgggggctcgagctggcagcagtgagagtatcgctgccttcatccaacgcctcagtggatccagcaacatctttgagcctggggctgatggcgcccttggattcttcggagctctgctctctctcctgtgccagaatttctccatggtggatgtggtgatgcttctccatggccatttccagccactgcagcggctccagccacagctgcgatctttcttccaccagcactacctgggtggccaggagcccacgcctagcaacatccggatggcgacccacacactgatcactgggctggaagagtatgtaagggagagtttttctttggtacaggttcagccaggtgtggatatcatccggacaaatttagaatttctccaagagcagtttaacagcattgctgctcatgtgctgcactgtacagacagtggatttggagcccggttgctggagctgtgtaaccagggcctgtttgaatgcttggccctgaacctccactgcttggggggacagcaaatggagcttgctgctgtcatcaatggtcgaattcgccgcatgtctcgcggggtgaatccatccttggtgagctggctgacaaccatgatgggactgaggcttcaggtggtcttggagcacatgcctgtgggtcccgacgccatcctcagatatgttcgtagggttggtgatcctcctcagacacttcctgaagagccgatggaagttcagggagcagaaagaacttcccctgaacctcagagagagaatgcttccccagcccctggaacaacagcagaagaagccatgtcccgaggcccgccccctgctcctgaaggaggttcccgagatgaacaggatggagcttcagctgatgcagaaccctgggcagctgcagtcccccctgaatgggtccctattatccagcaggacattcagagccagcggaaggtgaaacctcagccgcccctgagtgatgcctacctcagtggcatgcctgccaagagacgaaagacaatgcagggtgagggcccccagctgctactctcagaggcagtgagccgggcagctaaggcagccggagctcggcccctgacaagccccgagagcctgagccgggacctggaggcaccagaggttcaggagagctacaggcagcagctccggtctgatatccaaaaacgactgcaggaagatcccaactacagcccccagcgcttccctaatgcccatcgggcatttgctgatgacccctag

SEQ ID NO: 23 - protein, 1154 aa, MOUSE
MEPSDSASTAMEEPDSLEVLVKTLDSQTRTFIVGAQMNVKEFKEHIAASVSIPSEKQRLIYQGRVLQDDKKLQEYNVGGKVIHLVERAPPQTQLPSGASSGTGSASATHGGAPLPGTRGPGASVHDRNANSYVMVGTFNLPSDGSAVDVHINMEQAPIQSEPRVRLVMAQHMIRDIQTLLSRMECRGGTQAQASQPPPQTPQTVASETVALNSQTSEPVESEAPPREPMESEEMEERPPTQTPELAPSGPAPAGPAPAGPAPAPETNAPNHPSPAEHVEVLQELQRLQRRLQPFLQRYCEVLGAAATTDYNNNHEGREEDQRLINLVGESLRLLGNTFVALSDLRCNLACAPPRHLHVVRPMSHYTTPMVLQQAAIPIQINVGTTVTMTGNGARPPPAPGAEAATPGSAQATSLPPSSTTVDSSTEGAPPPGPAPPPASSHPRVIRISHQSVEPVVMMHMNIQDSGAQPGGVPSAPTGPLGPPGHGQTLGQQVPGFPTAPTRVVIARPTPPQARPSHPGGPPVSGALQGAGLGTNTSLAQMVSGLVGQLLMQPVLVAQGTPGMAQAQAQAQAQAQAQAQAPAPAPAPAPAPATASASAGTTNTATTAGPAPGGPAQPPPPQPSAADLQFSQLLGNLLGPAGPGAGGPGMASPTITVAMPGVPAFLQGMTDFLQASQTAPPPPPPPPPPPPAPEQQSTPPPGSPSGGTASPGGLGPESLPPEFFTSVVQGVLSSLLGSLGARAGSSESIAAFIQRLSGSSNIFEPGADGALGFFGALLSLLCQNFSMVDVVMLLHGHFQPLQRLQPQLRSFFHQHYLGGQEPTPSNIRMATHTLITGLEEYVRESFSLVQVQPGVDIIRTNLEFLQEQFNSIAAHVLHCTDSGFGARLLELCNQGLFECLALNLHCLGGQQMELAAVINGRIRRMSRGVNPSLVSWLTTMMGLRLQVVLEHMPVGPDAILRYVRRVGDPPQTLPEEPMEVQGAERTSPEPQRENASPAPGTTAEEAMSRGPPPAPEGGSRDEQDGASADAEPWAAAVPPEWVPIIQQDIQSQRKVKPQPPLSDAYLSGMPAKRRKTMQGEGPQLLLSEAVSRAAKAAGARPLTSPESLSRDLEAPEVQESYRQQLRSDIQKRLQEDPNYSPQRFPNAHRAFADDP

SEQ ID NO: 24 - DNA, 18 bp, Artificial Sequence
  Synthetic Primer
gccccgctcagacctagt

SEQ ID NO: 25 - DNA, 20 bp, Artificial Sequence
  Synthetic Primer
acgttctgtctcctgtgctg

(19) United States
(12) Patent Application Publication
(10) Pub. No.: US 2006/0159681 A1
(43) Pub. Date: Jul. 20, 2006

(54) Compositions and methods to inhibit cell loss by using inhibitors of BAG

(75) Inventors: Andres M. Lozano, Toronto (CA); Suneil K. Kalia, Toronto (CA)

Correspondence Address: FULBRIGHT & JAWORSKI, LLP, 1301 MCKINNEY, SUITE 5100, HOUSTON, TX, 77010-3095, US

(73) Assignee: Functional Neuroscience Inc., Toronto (CA)

(21) Appl. No.: 11/301,457

(22) Filed: Dec. 13, 2005

Related U.S. Application Data
(60) Provisional application No. 60/635,929, filed on Dec. 14, 2004.

(51) Int. Cl. A61K 48/00; A61K 39/395

(52) U.S. Cl. 424/143.1; 514/44

(57) ABSTRACT

The present invention relates to inhibitors of BAG and uses thereof, such as to inhibit cell loss, inhibit parkin sequestration, and/or inhibit formation of protein aggregates. More specifically, the present invention relates to BAG inhibitors used to inhibit neurodegeneration and to treat a neurodegenerative disease, such as Parkinson's disease.

### CROSS-REFERENCE TO RELATED APPLICATIONS

This application claims priority to U.S. Provisional Application No. 60/635,929 filed Dec. 14, 2004, which is incorporated herein by reference in its entirety.

### TECHNICAL FIELD

The present invention relates to the field of cell biology. More specifically, it relates to inhibitors of BAG and their uses thereof, for example the use of a BAG inhibitor to inhibit cell loss, such as neuronal cell loss. Yet further, the present invention relates to inhibitors of BAG5 to inhibit parkin sequestration, inhibit formation of protein aggregates, inhibit neurodegeneration and/or to treat a neurodegenerative disease, such as Parkinson's disease, for example.

### BACKGROUND OF THE INVENTION

Parkinson's disease (PD) is a common neurodegenerative disease characterized by the progressive loss of dopaminergic neurons in the substantia nigra pars compacta (SNpc) (Lang and Lozano, 1998). Protein aggregates known as Lewy Bodies (LBs) are pathological hallmarks of PD, and protein aggregation is a common feature among many neurodegenerative diseases (Lang and Lozano, 1998; Sherman and Goldberg, 2001). Recently, genes linked to hereditary PD have been identified including alpha-synuclein, DJ-1, PINK1 and two components of the ubiquitin-proteasome system (UPS): ubiquitin carboxy-terminal hydrolase L1 (UCH-L1) and parkin (PARK2) (Vila and Przedborski, 2004). Current evidence implicates the impaired regulation of protein aggregation and dysfunction of the UPS as a common pathway in the progression of both genetic and sporadic forms of PD (Giasson and Lee, 2003; Sherman and Goldberg, 2001).

The UPS mediates the ubiquitinylation of a substrate by a multi-step enzymatic process that includes an ubiquitin-activator (E1), an ubiquitin-conjugator (E2), and an ubiquitin-ligase (E3). Ubiquitinylated substrates may then be targeted for degradation by the proteasome (Hershko and Ciechanover, 1998). Parkin is an E3 ubiquitin-ligase (Imai et al., 2000; Shimura et al., 2000; Zhang et al., 2000) and mediates the ubiquitinylation of itself (Staropoli et al., 2003; Zhang et al., 2000) and various protein substrates (Chung et al., 2001; Corti et al., 2003; Huynh et al., 2003; Imai et al., 2001; Ren et al., 2003; Shimura et al., 2001; Staropoli et al., 2003; Tsai et al., 2003; Zhang et al., 2000). A subset of mutations associated with autosomal recessive Juvenile Parkinson's Disease (AR-JPD) results in the loss of parkin E3 function (Imai et al., 2001; Imai et al., 2000; Shimura et al., 2000; Zhang et al., 2000), which likely leads to UPS dysfunction and the accumulation of parkin substrates resulting in neurodegeneration (Giasson and Lee, 2003). Indeed, overexpression of parkin suppresses neurodegeneration associated with UPS dysfunction (Petrucelli et al., 2002) or endoplasmic reticulum (ER) stress (Imai et al., 2001; Yang et al., 2003), whereas targeted decreases in parkin expression augment cell death (Petrucelli et al., 2002; Yang et al., 2003).

In brain, parkin associates with the molecular chaperone Heat shock protein 70 (Hsp70) (Imai et al., 2002) and both Hsp70 (Auluck et al., 2002; McLean et al., 2002) and parkin (Schlossmacher et al., 2002) are found in LBs in sporadic PD. Hsp70 prevents the formation of protein aggregates (Adachi et al., 2003; Chan et al., 2000; Muchowski et al., 2000) and is neuroprotective in both Drosophila (Auluck et al., 2002; Chan et al., 2000; Warrick et al., 1999) and mammalian models (Adachi et al., 2003; Cummings et al., 2001) of neurodegenerative disease.

The role of parkin and Hsp70 in sporadic PD is not clearly understood. However, it is possible that loss of parkin (Ardley et al., 2003; Winklhofer et al., 2003) and Hsp70 (Sherman and Goldberg, 2001) function may occur through their sequestration in LBs as a result of UPS dysfunction and cellular stress. It is also possible that negative modulators of parkin and Hsp70 exist and contribute to the pathogenesis of sporadic PD. Indeed it has been recently shown that S-nitrosylation of parkin results in the inhibition of parkin E3 activity both in vitro and in vivo providing a possible mechanistic link between the genetic and sporadic forms of PD (Chung et al., 2004; Yao et al., 2004).

BAG1, an Hsp70 interacting protein that contains a prototypic BAG domain at its C-terminus, was cloned based on its synergistic interaction with bcl-2 (Takayama et al., 1995) and is generally considered to confer resistance to cell death (Takayama and Reed, 2001). BAG1 functions as a co-chaperone of Hsp70 through the interaction of its BAG domain and the ATPase domain of Hsp70 (Briknarova et al., 2001; Hohfeld and Jentsch, 1997; Sondermann et al., 2001; Takayama et al., 1997; Zeiner et al., 1997). There are currently six known human BAG-family members (BAG1-6), and homologues have been identified in yeast, invertebrates, mammals and plants (Takayama and Reed, 2001). BAG5 is the only BAG-family member predicted to contain multiple BAG domains and currently remains uncharacterized. Aside from the formation of the BAG-Hsp70 complex, BAG proteins functionally interact with a variety of binding partners and coordinate diverse cellular processes, such as stress signaling (Song et al., 2001), cell division, cell death and cell differentiation (Takayama and Reed, 2001), for example.

The present invention is the first to describe the use of BAG inhibitors (such as the exemplary BAG5) to attenuate or decrease cell death or apoptosis and/or treat diseases associated with cell death and protein aggregation, such as neurodegenerative diseases, for example.

### BRIEF SUMMARY OF THE INVENTION

The present invention relates to the identification of BAG inhibitors and the uses thereof to attenuate cell loss. The inhibitors can be used, for example, to attenuate cell loss, neurodegeneration, the loss of neuronal cells, apoptosis of neuronal cells, and/or the progressive or acute loss of neuronal function. The inhibitors may be employed to attenuate or inhibit protein aggregation, in particular aspects of the invention. BAG inhibitors or related compounds of the present invention can be used to increase or enhance cell survival, and in these specific embodiments the inhibitors of the present invention may be considered to be protective. The compositions and methods of the invention may be employed as therapeutic and/or preventative embodiments.

An embodiment of the present invention is a method of attenuating cell loss comprising the step of administering to the cell a bcl-2 associated athanogene (BAG) inhibitor, wherein the BAG inhibitor decreases expression or activity of BAG thereby attenuating cell loss.

Cells types that can be used in the present invention may be any type, although in specific embodments they include blood cells, immune cells, muscle cells, nerve or neuronal cells, or gland cells, for example. More specifically, cells that can be associated with the invention can be isolated from the following exemplary tissues: ameloblasts, axon, basal cells, blood (e.g., lymphocytes), blood vessel, bone, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, prostate, skin, small intestine, spleen, stem cells, stomach, and/or testes.

In certain embodiments, the cell loss comprises neuronal cell loss, and the neuronal cells may be selected from the group consisting of cholinergic, adrenergic, noradrenergic, dopaminergic, serotonergic, glutaminergic, GABAergic, and glycinergic. More particularly, the cell loss is associated with neurodegeneration, and in further specific embodiments the cell is a dopaminergic cell.

The BAG family comprises several members, for example, but is not limited to BAG1, BAG2, BAG3, BAG4, BAG5 and/or BAG6, and/or derivatives thereof. Thus, in the present invention an inhibitor is an inhibitor that attenuates or inhibits the activity and/or expression of at least one of any BAG family member. Such inhibitors can comprise a nucleic acid molecule, a protein (e.g., an antibody), a small molecule, derivatives thereof, or combinations thereof.

In certain embodiments, the BAG inhibitor comprises a nucleic acid molecule and/or protein that inhibits the expression and/or activity of BAG. More specifically, the BAG inhibitor is nucleic acid molecule, for example, a mutant BAG nucleic acid, including a mutant BAG5 nucleic acid molecule (SEQ. ID. NO. 9), for example, that encodes a polypeptide having the sequence of SEQ. ID. NO. 8. Still further, the BAG inhibitor is an antisense molecule, more specifically, an siRNA molecule or an shRNA molecule, for example, the shRNA molecule that is encoded by the nucleic acid sequence of SEQ. ID. NO. 7.

In further embodiments, the nucleic acid sequence is comprised in an expression vector, for example, a viral or a non-viral vector, such as a plasmid vector. The viral vector may be an adenoviral vector, an adeno-associated viral vector, a retroviral vector, a lentiviral vector, a herpes viral vector, polyoma viral vector or a hepatitis B viral vector, for example. Yet further, the expression vector can be in a non-viral delivery system, for example, a non-viral delivery system that comprises one or more lipids.

Still further, the BAG inhibitor may be prepared by the process of designing or selecting a candidate substance suspected of having the ability of decreasing BAG activity or BAG expression. The inhibitor increases HSP-70 chaperone activity, increases parkin E3 ubiquitin-ligase activity, decreases sequestration of parkin, and/or decreases protein aggregation, in specific embodiments.

Yet further, the cell may be in a subject suffering from a neurodegenerative disease, wherein the subject is a human, such as a human patient, for example. The neurodegenerative disease may be selected from the group consisting of Alzheimer's Disease, Parkinson's Disease, Huntington's Disease, amyotrophic lateral sclerosis (ALS), Guillain-Barre syndrome, multiple sclerosis, epilepsy, myasthenia gravis, chronic idiopathic demyelinating disease (CID), neuropathy, ataxia, dementia, chronic axonal neuropathy, and/or stroke. More specifically, the neurodegenerative disease is Parkinson's Disease, preferably, sporadic Parkinson's Disease.

Thus, in the present invention, blocking cell loss and/or enhancing neuroprotection by removing the deleterious consequences of BAG activity can have several applications. This form of therapy can be used in any medical condition wherein at least one symptome of ht econditions is ameliorated at least in part. For example, it may be employed in a number of neurological and psychiatric disorders affected by neuronal loss and where there can be clinical benefit from decreasing neuronal loss. For example, targeting the hippocampal neuronal loss can be important in depression, epilepsy, post cranial irradiation, steroid induced impairment in neurogenesis, stress disorders, cognitive disorders, and/or in Alzheimer's disease, for example. Targeting the cortical, striatal, substantia nigra, brainstem and cerebellar loss is important in Huntington's Disease, Alzheimers, multiple system atrophy, Parkinson's disease, post-cranial irradiation disorders, paraneoplastic disorders, and the Spinocerebellar ataxias, for example. This method may also be applicable to treat the neuronal loss that occurs as a consequence of a number of disorders including, but not limited to, congenital disorders, stroke, anoxia, hypoxia, stroke, hypoglycemia, metabolic disorders, head injury, drug and alcohol toxicity, nutritional deficiencies, auto-immune infectious and inflammatory processes including Multiple sclerosis, Guillain-Barre syndrome, chronic idiopathic demyelinating neuropathy (CID), neuropathy, ataxias and myasthenia gravis, for example.

Still further, the present invention comprises an isolated nucleic acid sequence that encodes an siRNA molecule or that encodes a mutant polypeptide. Using the nucleic acid sequence of any BAG-family protein and the publicly available siRNA techniques of obtaining siRNA molecules, or techniques that are available for producing mutant polypeptides, one of skill in the would be able to obtain such nucleic acid sequences encoding BAG siRNA molecules or nucleic sequences encoding a mutant BAG polypeptide. An exemplary nucleic acid sequence that encodes an BAG5 siRNA molecule may include the sequence of SEQ. ID. NO. 7; and an exemplary mutant BAG5 nucleic sequence is SEQ. ID. NO. 9. The nucleic sequence may be comprised in an expression vector, for example, a viral or plasmid vector. The viral vector may be an adenoviral vector, an adeno-associated viral vector, a retroviral vector, a lentiviral vector, a herpes viral vector, polyoma viral vector or hepatitis B viral vector. In other embodiments, the isolated nucleic acid sequence that encodes a BAG5 siRNA molecule or mutant BAG5 nucleic acid molecule may be comprised in a non-viral delivery system, for example, the non-viral delivery system that comprises one or more lipids.

Another embodiment of the invention includes a method of making a BAG inhibitor comprising: (a) providing a candidate substance suspected of decreasing BAG expression or activity; (b) selecting the BAG inhibitor by assessing the ability of the candidate substance to decrease BAG expression or activity; and (c) making the selected BAG inhibitor. The candidate substance may be a protein, a nucleic acid molecule, an organo-pharmaceutical, or a combination thereof, for example. Still further, the providing step may be further defined as providing in a cell or a cell-free system a BAG polypeptide, and the BAG polypeptide is contacted with the candidate substance.

Still further, another embodiment of the present invention is a pharmaceutical composition comprising an inhibitor that is in accordance with the present invention, such as made by any of the methods described herein or suitable in the art. Still further, another embodiment is a pharmaceutical composition comprising an inhibitor of the present invention admixed with a pharmaceutical carrier. The inhibitor can be a protein, a nucleic acid molecule, an organo-pharmaceutical, or a combination thereof. More specifically, the inhibitor is a nucleic acid molecule. More particularly, the nucleic acid molecule is an antisense molecule and/or an siRNA molecule and/or a mutated nucleic acid molecule that encodes a mutated protein or polypeptide. In certain embodiments, the inhibitor that is admixed with a pharmaceutical carrier comprises an siRNA molecule that is encoded by a nucleic acid sequence further defined as SEQ. ID. NO. 7. Still further, the inhibitor that is admixed with a pharmaceutical carrier comprises a mutated nucleic acid molecule that is encoded by SEQ. ID. NO. 9.

In additional embodments, a kit comprising one or more compositions of the invention is contemplated. The kit may comprise a BAG inhibitor and, in some embodiments, an additional agent that treats and/or prevents at least one symptom of neuronal loss, such as with a neurodegenerative disease. The components of the kit will be housed in suitable container(s). In specific aspects the kit comprises one or more apparatuses to deliver the composition(s). Exemplary apparatuses in the kit include a syringe, spoon, or cup.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A-1E show the cloning and characterization of BAG5, as an exemplary BAG. FIG. 1A shows the sequence of cloned rat BAG5 aligned with human and mouse sequences. The four BAG domains are shaded and a putative “short” fifth BAG domain (Briknarova et al., 2002) is underlined. Substituted residues in BAG5(DARA) are indicated with an asterisk (*). FIG. 1B shows Northern blots of BAG5 expression in rat and human. FIG. 1C shows in situ hybridization (ISH) of adult rat brain using a BAG5-specific anti-sense radiolabeled probe. The lower panel is a representative ISH using a control sense probe. FIG. ID shows combined ISH and TH immunohistochemistry (IHC) in SNpc of rat brain using a DIG-BAG5 probe shows TH+ neurons are also BAG5-mRNA+ (arrows, upper panels). The lower panels show a representative combined ISH and IHC using a control sense BAG5 probe. The upper and middle panels are from the SNpc ipsilateral and contralateral to MFB axotomy (MFBx) respectively. FIG. 1E shows quantitative analysis of ISH signal density in the SNpc at 24 hours post axotomy.

FIGS. 2A-2H show that BAG5 protein is expressed in the SNpc of rodent and human brain. FIG. 2A shows a Western blot (WB) of transfected SH-SY5Y lysate (control (−) or pDEST-FLAG-BAG5 vector (+)) probed with anti-BAG5 and anti-FLAG. Pre-absorption of anti-BAG5 antibody with BAG5 peptide immunogen is shown on the right. FIG. 2B shows WB of mouse brain and human brain lysates using the anti-BAG5 antibody. FIG. 2C shows IHC of human cortex with anti-BAG5 (left panel) or preabsorbed anti-BAG5 (right panel). IHC of mouse (FIG. 2D) and rat (FIG. 2E) SNpc shows BAG5 immunoreactivity (green) co-localized with TH+-neurons (red). BAG5 immunoreactivity co-localizes with TH immunoreactivity throughout the SNpc and ventral tegmental area (VTA) of midbrain ((FIG. 2E), right panel). FIG. 2F shows BAG5 immunoreactivity (green) is co-localized in TH+ neurons (red) in the SNpc from a patient with DLB. FIG. 2G shows BAG5 immunoreactivity (green) co-localizes in alpha-synuclein+ neurons (red) in human DLB SNpc. FIG. 2H shows BAG5 immunoreactivity is also present in alpha-synuclein+perinuclear Lewy Bodies. Scale bars, 50 μm (FIG. 2C), 10 μm (FIGS. 2D, E), and 20 μm (FIG. 2F-H). Staining in (FIG. 2D-H) is the nuclear-stain Hoechst33342. Molecular weight markers are shown in kDa.

FIGS. 3A-3G show that BAG5 is a co-chaperone of Hsp70. FIG. 3A shows GST-BAG5 pull-down assay with His-Hsp70, His-Hsp70 ATPase domain, His-Hsp70 PBD or human brain SNpc lysate (400 μg). FIG. 3B shows GST-Hsp70 pull-down assay from human brain SNpc lysate shows Hsp70 binding to endogenous BAG5. FIG. 3C shows xo-immunoprecipitation of endogenous Hsp70 and BAG5 from human brain lysate (2 mg). FIG. 3D shows luciferase refolding assay in HEK293T cells. FIG. 3E shows that BAG5 associates with Hsp70 and itself as shown by co-immunoprecipitation of GFP-BAG5 and FLAG-BAG5 from transfected HEK293T lysate. The asterisk (*) indicates the immunoglobulin heavy chain. FIG. 3F shows GST-BAG5 and GST-BAG5(DARA) pull-down assays from HEK293T cell lysates transfected with GFP, GFP-BAG5 or GFP-BAG5(DARA). Inputs are shown in the lower Western Blot (WB). FIG. 3G shows that luciferase refolding assay in HEK293T cells, and shows BAG5(DARA) mitigates BAG5 inhibition of Hsp70-mediated refolding of thermally inactivated luciferase. Representative WBs are shown above the graph.

FIGS. 4A-4G show that BAG5 interacts directly with parkin and inhibits parkin E3 activity. FIG. 4A shows BAG5 associates with both parkin and Hsp70 as shown by co-immunoprecipitation from lysates of transfected HEK293T cells. FIG. 4B shows GST pull-down assay demonstrating BAG5, BAG5(DARA) and Hsp70 directly interact with His-parkin. FIG. 4C shows schematic of parkin deletion constructs. FIG. 4D shows GST-BAG5 pull-down assays from HEK293T cell lysate transfected with indicated GFP-parkin deletion constructs. FIG. 4E shows in vitro ubiquitinylation assay shows BAG5 inhibits parkin auto-ubiquitinylation. FIG. 4F shows ubiquitinylation assay in transfected HEK293T cells shows parkin enhances the ubiquitinylation of synphilin (FLAG-sphl). FIG. 4G shows biquitinylation assays in transfected HEK293T cells show both BAG5 and BAG5(DARA) inhibit parkin-mediated ubiquitinylation of the substrate synphilin.

FIGS. 5A-5G show that BAG5 prevents Hsp70-mediated inhibition of the formation of parkin containing protein aggregates. FIG. 5A shows Parkin is sequestered in protein aggregates following proteasome inhibition by MG132 in HEK293T cells (arrows). FIG. 5B shows Parkin containing protein aggregates are also immunoreactive for BAG5 and ubiquitin (left panels) and are surrounded by immunoreactivity for Hsp70 (right panels). Scale bars=10 μm. FIG. 5C shows BAG5 inhibits Hsp70-mediated prevention of parkin aggregation whereas BAG5(DARA) does not. FIG. 5D shows the mutant BAG5(DARA) mitigates BAG5-mediated inhibition of Hsp70 in the parkin aggregation assay. Corresponding representative WBs are shown above the graphs in (FIG. 5C) and (FIG. 5D). FIG. 5E shows U6_shRNA_b5 depresses FLAG-huBAG5 protein expression but is unable to depress FLAG-rtBAG5 expression as shown by WBs. Lower panels show U6_shRNA_b5 depresses GFP-BAG5 expression in HEK293T cells whereas empty vector (Control) and an unrelated shRNA (U6_shRNA_control) do not. FIG. 5F shows U6_shRNA_b5 but not U6_shRNA_control depresses endogenous BAG5 mRNA and protein expression in HEK293T cells as shown by RT-PCR and WB. FIG. 5G shows U6_shRNA_b5-mediated depression of BAG5 significantly decreases the relative sequestration of parkin in protein aggregates whereas U6_shRNA_control does not. Protein aggregation in the graph is relative to aggregation of GFP-parkin alone.

FIGS. 6A-6E show that BAG5 mitigates parkin-mediated inhibition of proteasome stress and cell death. FIG. 6A shows synphilin (sphl) enhances the accumulation of GFPu (upper panels). DsRed (middle panels) is a transfection control and Hoechst 33342 (lower panels) shows total cell numbers in the same field. FIG. 6B shows that WB shows a synphilin-mediated dose-dependent increase in GFPu accumulation. FIG. 6C shows BAG5 prevents parkin-mediated rescue of GFPu accumulation due to synphilin. FIG. 6D shows BAG5(DARA) mitigates the effect of BAG5 on GFPu accumulation. Graphs in (FIG. 6C) and (FIG. 6D) show beta-galactosidase normalized GFPu signal and corresponding WB. Corresponding WBs of each condition are shown above the graph. FIG. 6E shows BAG5 prevents parkin-mediated inhibition of cell death. Graphs show cell death observed in SH-SY5Y cells transfected with sphl and synuclein (syn) and treated with the irreversible proteasome inhibitor lactacystin (15 μM).

FIGS. 7-7G show that adenovirus-mediated expression of BAG5 in the SNpc enhances dopaminergic neurodegeneration post-axotomy and in the MPTP model of PD. FIG. 7A shows WB of lysates of SH-SY5Y infected with Ad.BAG5 and Ad.BAG5(DARA). FIG. 7B shows IHC showing expression of FLAG-BAG5(DARA) and FLAG-BAG5 in the SNpc of rats ipsilateral to the adenovirus injected striatum and not in the contralateral SNpc (Control). FIG. 7C shows TH immunoreactivity in control SNpc and lesioned SNpc 3 days and 7 days post-axotomy in rats injected with adenovirus. FIG. 7D shows quantitation of TH positive neurons as a percent of the contralateral unlesioned. FIG. 7E shows IHC showing expression of adenovirus in the SNpc of mice ipsilateral to the injected striatum and not in the contralateral SNpc (Control). FIG. 7F shows TH immunoreactivity in the SNpc ipsilateral to injection with adenovirus in mice at 2 weeks following treatment with saline or MPTP. FIG. 7G shows quantitation of TH positive neurons in the adenovirus injected SNpc relative to contralateral SNpc.

FIG. 8 shows an exemplary model for the role of BAG5 in enhancing neurodegeneration.

### DETAILED DESCRIPTION OF THE INVENTION

I. Definitions

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

As used herein, the term “BAG inhibitor” refers to an inhibitor of any BAG-family member, for example, but not limited to BAG1, BAG2, BAG3, BAG4, BAG5, and BAG. One of skill in the art is aware that other synonyms for BAG6 include, G3, Scythe, and D17H6S52E. An inhibitor of one BAG may inhibit one or more other BAG, in specific embodiments, and one of skill in the art is aware how to test inhibition by suitable methods, such as those described herein. The inhibitor may inhibit at least in part the activity and/or expression of at least one BAG or derivative thereof. The term “inhibitor” as used herein refers to a molecule or compound that acts to suppress the expression or function of another compound. More specifically, the “inhibitor” decreases the biological activity of a gene, an oligonucleotide, protein, enzyme, inhibitor, signal transducer, receptor, transcription activator, co-factor, and the like. Such inhibition may be contingent upon occurrence of a specific event, such as activation of a signal transduction pathway and/or may be manifest only in particular cell types. In specific embodiments, inhibitor decreases the ability of a BAG-family protein to interact with Hsp70 and/or more specifically, to decrease the ability of BAG5 to interact with parkin. Thus, the inhibition of BAG activity or expression decreases cell loss (e.g., neurodegeneration or neuronal cell loss), attenuates parkin sequestration and protein aggregation, and enhances Hsp70 chaperone activity.

The term “effective amount” as used herein is defined as an amount of the agent that will decrease, reduce, inhibit or otherwise abrogate the degeneration or death of neuronal cells. Thus, an effective amount is an amount sufficient to detectably and repeatedly ameliorate, reduce, minimize or limit the extent of the disease or its symptoms.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, the term “cell loss” refers to the loss of cells, or cell death, or cell apoptosis.

As used herein, the term “expression construct” or “transgene” is defined as any type of genetic construct containing a nucleic acid coding for gene products in which part or all of the nucleic acid-encoding sequence that is capable of being transcribed can be inserted into the vector. The transcript is translated into a protein, but it need not be. For example, the expression construct may comprise nucleic acid sequences encoding antisense molecules or siRNA or shRNA molecules. In certain embodiments, expression includes both transcription of a gene and translation of mRNA into a gene product. In other embodiments, expression only includes transcription of the nucleic acid encoding polynucleotides (which may be referred to as genes, in specific embodiments) of interest. In the present invention, the term “therapeutic construct” may also be used to refer to the expression construct or transgene. One skilled in the art realizes that the present invention utilizes the expression construct or transgene as a therapy to treat and/or prevent neurodegenerative diseases or disorders, such as cancer; thus, the expression construct or transgene is a therapeutic construct and/or a prophylactic construct.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes or siRNA or shRNA (short-hairpin RNA). Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described infra. In the present invention, the term “therapeutic vector” may also be used to refer to the expression vector. One skilled in the art realizes that the present invention utilizes the expression vector as a therapy to treat neurodegenerative diseases.

As used herein, the term “gene” is defined as a functional protein, polypeptide, or peptide-encoding unit. As will be understood by those in the art, this functional term includes genomic sequences, cDNA sequences, and smaller engineered gene segments that express, or is adapted to express, proteins, polypeptides, domains, peptides, fusion proteins, and mutants.

As used herein, the term “neuronal” or “neuron” refers to one or more cells that are a morphologic and functional unit of the brain, spinal column, and peripheral nerves consisting of nerve cell bodies, dendrites, and axons. Neuron cell types can include, but are not limited to, typical nerve cell body showing internal structure, horizontal cell from cerebral cortex, Martinotti cell, biopolar cell, unipolar cell, Purkinje cell, and pyramidal cell of motor area of cerebral cortex. Exemplary neuronal cells can include, but are not limited to, cholinergic, adrenergic, noradrenergic, dopaminergic, serotonergic, glutaminergic, GABAergic, and glycinergic.

As used herein, the term “neurodegenerative disease” or “degenerative disease” is defined as a disease or condition in which there is a progressive loss of neurons or loss of neuronal function. Thus, a neurodegenerative disease, as used in the current context, should be obvious to one skilled in the art, but is meant to include any abnormal physical or mental behavior or experience where the death of neuronal cells is involved in the etiology of the disorder, or is affected by the disorder. As used herein, neurodegenerative diseases encompass disorders affecting the central and peripheral nervous systems, and include such afflictions as memory loss, stroke, dementia, personality disorders, gradual, permanent or episodic loss of muscle control. Examples of neurodegenerative diseases for which the current invention can be used preferably include, but are not limited to, Alzheimer's Disease, Parkinson's Disease, Huntington's Disease, amyotrophic lateral sclerosis (ALS), Guillain-Barre syndrome, multiple sclerosis, epilepsy, myasthenia gravis, chronic idiopathic demyelinating disease (CID), neuropathy, ataxia, dementia, chronic axonal neuropathy and stroke.

As used herein, the term “neurodegeneration” refers to the progressive loss or function of at least one neuron or neuronal cell. One of skill in the art realizes that the term progressive loss can refer to cell death or cell apoptosis.

As used herein, the term “neuronal cell loss” refers to the loss of neuronal cells. The loss of neuronal cells may be a result of a genetic predisposition, congenital dysfunction, apoptosis, ischemic event, immune-mediated, free-radical induced, chemical induced, or any injury that results in a loss of neuronal cells, as well as a progressive loss of neuronal cells.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, one skilled in the art is cognizant that polynucleotides include mutations of the polynucleotides, including but not limited to, mutation of the nucleotides or nucleosides by methods well known in the art.

As used herein, the term “polypeptide” is defined as a chain of amino acid residues, usually having a defined sequence. As used herein, the term polypeptide is interchangeable with the terms “peptides” and “proteins”.

As used herein, the term “siRNA” is meant to be equivalent to other terms used to describe nucleic acid molecules that are capable of mediating sequence-specific RNAi, for example short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

As used herein, the term “RNAi” is meant to be equivalent to other terms used to describe sequence-specific RNA interference, such as post transcriptional gene silencing, translational inhibition, or epigenetics. For example, siRNA molecules of the invention can be used to epigenetically silence genes at both the post-transcriptional level or the pre-transcriptional level.

The term “treating” and “treatment” as used herein refers to administering to a subject an effective amount of an inhibitor of BAG so that the subject has an improvement in the disease or condition, such as neuronal cell loss. The improvement is any improvement or remediation of the symptoms. The improvement is an observable or measurable improvement. Thus, one of skill in the art realizes that a treatment may improve at least one symptom of the disease condition, but may not be a complete cure for the disease.

The term “variant” or “variants” as used herein refers to polynucleotides or polypeptides that differ from a reference polynucleotide or polypeptide respectively. Variants in this sense are described below and elsewhere in the present disclosure in greater detail. For example, changes in the nucleotide sequence of the variant may be silent, i.e., they may not alter the amino acids encoded by the polynucleotide. Where alterations are limited to silent changes of this type a variant will encode a polypeptide with the same amino acid sequence as the reference polypeptide. Changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence, as discussed below. Generally, differences in amino acid sequences are limited so that the sequences of the reference and the variant are closely similar overall and, in many regions, identical. A variant and reference polypeptide may differ in amino acid sequence by one or more substitutions, additions, deletions, fusions and truncations, which may be present in any combination. A variant may also be a fragment of a polynucleotide or polypeptide of the invention that differs from a reference polynucleotide or polypeptide sequence by being shorter than the reference sequence, such as by a terminal or internal deletion. For example, a variant may be a result of alternative mRNA splicing. Alternative mRNA splicing can lead to tissue-specific patterns of gene expression by generating multiple forms of mRNA that can be translated into different protein products with distinct functions and regulatory properties. These splice variants may occur by a mutation in an intron or exon or may be influenced post-transcriptionally. For example, the gene for inducible nitric-oxide synthase has the capacity to generate four mRNA isoforms by alternative mRNA splicing. These alternatively spliced mRNA transcripts are regulated in a tissue-specific manner and induced by cytokines (Eissa et al., 1996). Another variant of a polypeptide of the invention also includes a polypeptide which retains essentially the same function or activity as such polypeptide, e.g., proproteins which can be activated by cleavage of the proprotein portion to produce an active mature polypeptide.

A variant may also be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide or a proprotein sequence. A variant of the polynucleotide or polypeptide may be a naturally occurring variant such as a naturally occurring allelic variant, or it may be a variant that is not known to occur naturally. Such non-naturally occurring variants of the polynucleotide may be made by mutagenesis techniques, including those applied to polynucleotides, cells or organisms, or may be made by recombinant means. Among polynucleotide variants in this regard are variants that differ from the aforementioned polynucleotides by nucleotide substitutions, deletions or additions. The substitutions, deletions or additions may involve one or more nucleotides. The variants may be altered in coding or non-coding regions or both. Alterations in the coding regions may produce conservative or non-conservative amino acid substitutions, deletions or additions. In specific embodiments, a variant may be further defined as a derivative. All such variants defined above are deemed to be within the scope of those skilled in the art from the teachings herein and from the art.

II. Present Invention

The present invention relates to inhibitors of BAG family proteins or polynucleotides, such as genes. An exemplary BAG, BAG5, is a previously uncharacterized BAG domain-containing family member that interacts with both Hsp70 and parkin with deleterious functional consequences. Through these interactions BAG5 inhibits Hsp70 chaperone activity, parkin E3 ubiquitin-ligase activity, and enhances the sequestration of parkin within LB-like protein aggregates. In contrast, a BAG5 inhibitor, for example, short-hairpin RNA (shRNA)-mediated depression of BAG5 expression results in decreases in the sequestration of parkin within protein aggregates. The present invention also indicates that targeted expression of BAG5 in a model of PD enhances dopaminergic neurodegeneration, whereas a mutant form of BAG5 that can inhibit BAG5 function results in enhanced cell survival.

In addition to BAG5 inhibitors, inhibitors of the other BAG proteins can also be used in the present invention. It is known that BAG-family proteins contain a conserved BAG domain near their C-terminus that binds Hsc70/Hsp70. Thus, the present invention relates to the identification of BAG inhibitors and the uses thereof. The inhibitors can be used to attenuate loss of cells and/or protein aggregation. More specifically, the inhibitors can be used to attenuate neurodegeneration or the loss of neuronal cells or apoptosis and/or the progressive or acute loss of neuronal function. BAG inhibitors or related compounds of the present invention can be used to increase or enhance cell survival, more specifically neuronal cell survival, thus the inhibitors of the present invention are protective or neuroprotective.

III. BAG Inhibitors

The Bcl-2 associated athanogene (BAG) contains several members, for example, BAG1, BAG2, BAG3, BAG4, BAG5, and BAG6. It is known that the BAG-family members interact with Hsc70/Hsp70 and this domain, which is highly conserved, is located near the C-terminus.

In certain embodiments, inhibitors of a BAG-family protein, for example, BAG1, BAG2, BAG3, BAG4, BAG5 and BAG6, are administered to a subject to reduce or inhibit the activity and/or expression of BAG. This reduction in BAG expression or activity is protective for cells or enhances cell survival. If the cells are neuronal cells, then the reduction in BAG expression or activity is considered neuroprotective.

The inhibitors of the present invention include, but are not limited to polynucleotides (RNA, DNA, or mixtures or combinations thereof), polypeptides, antibodies, small molecules or other compositions that are capable of inhibiting either the activity and/or the expression of a BAG-family related protein or gene (e.g., BAG1, BAG2, BAG3, BAG4, BAG5, and BAG6).

In the present invention, the term “BAG gene product” refer to proteins and polypeptides having amino acid sequences that are substantially identical to the native BAG-family amino acid sequences, for example, BAG1, BAG2, BAG3, BAG4, BAG5 or BAG6 (or RNA, if applicable) or that are biologically active, in that they are capable of performing functional activities similar to an endogenous BAG-family protein and/or cross-reacting with an anti-BAG antibody raised against a BAG-family protein.

The term “BAG gene product” also include related-compounds of the respective molecules that exhibit at least some biological activity in common with their native counterparts. Such related-compounds include, but are not limited to, truncated polypeptides and polypeptides having fewer amino acids than the native polypeptide. BAG gene product refers to any BAG-family member, for example, the BAG5 polypeptide sequences include, but are not limited to SEQ.ID.NO. 1 (GenBank®® accession AAR13081), SEQ.ID.NO. 2 (GenBank® accession NP_{—}004864), and SEQ.ID.NO. 3 (GenBank® accession No. XP_{—}127149). Sequences for other BAG-family related proteins include, but are not limite to BAG1 SEQ ID NO. 11 (GenBank® accession number AAC34258); BAG1 SEQ ID NO. 13 (GenBank® accession number NP_{—}004314); BAG2 SEQ ID NO. 15 (GenBank® accession No. NP_{—}004273); BAG3 SEQ ID NO 17 (GenBank® accession No. NP_{—}004272); BAG4 SEQ ID NO. 19 (GenBank® accession No. NP_{—}004865); BAG6 SEQ ID NO 21 (GenBank® accession No. AAC83822); BAG6 SEQ ID NO. 23 (GenBank® accession No. NP_{—}476512). BAG5 and other BAG gene products (BAG1, BAG2, BAG3, and BAG4), which can also be used in the present are more fully described in U.S. Pat. No. 6,696,558, which is incorporated herein by reference in its entirety.

The term “BAG gene” “BAG polynucleotide” or “BAG nucleic acid” refers to at least one molecule or strand of DNA (e.g., genomic DNA, cDNA) or RNA sequence (antisense RNA, RNAi, siRNA, shRNA) a derivative or mimic thereof, comprising at least one nucleotide base, such as, for example, a naturally occurring purine or pyrimidine base found in DNA (e.g., adenine “A,” guanine “G,” thymine “T,” and cytosine “C”) or RNA (e.g., A, G, uracil “U,” and C). The term “nucleic acid” encompasses the terms “oligonucleotide” and “polynucleotide.” These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. An “isolated nucleic acid” as contemplated in the present invention may comprise transcribed nucleic acid(s), regulatory sequences, coding sequences, or the like, isolated substantially away from other such sequences, such as other naturally occurring nucleic acid molecules, regulatory sequences, polypeptide or peptide encoding sequences, etc.

More particularly, a “BAG gene or BAG polynucleotide” may also comprise any combination of associated control sequences. One of skill in the art realizes that any BAG-family member can be used in the present invention. In certain embodiments, the exemplary BAG5 polynucleotide sequences include, but are not limited to SEQ.ID.NO. 4 (GenBank® accession No. AY366364), SEQ.ID.NO.5 (GenBank® accession No. NM_{—}004873) and SEQ.ID.NO. 6 (GenBank® accession No. XM_{—}127149). Sequences for other BAG-family related proteins include, but are not limited to BAG1 of SEQ ID NO. 10 (GenBank® accession number AF022224); BAG1 of SEQ ID NO. 12 (GenBank® accession number NM_{—}004323); BAG2 of SEQ ID NO. 14 (GenBank® accession No. NM_{—}004282); BAG3 of SEQ ID NO 16 (GenBank® accession No. NM_{—}004281); BAG4 of SEQ ID NO. 18 (GenBank® accession No. NM_{—}004874); BAG6 of SEQ ID NO 20 (GenBank® accession No. AF098511); and BAG6 of SEQ ID NO. 22 (GenBank® accession No. NM_{—}057171). BAG5 gene or polynucleotide is also described in U.S. Pat. No. 6,696,558, which is incorporated herein by reference in its entirety, and also describes other BAG gene or polynucleotides, such as BAG1, BAG2, BAG3, and BAG4. Thus, nucleic acid compositions encoding BAG, more specifically BAG5, are herein provided and are also available to a skilled artisan at accessible databases, including the National Center for Biotechnology Information's GenBank® database and/or commercially available databases, such as from Celera Genomics, Inc. (Rockville, Md.). Also included are splice variants that encode different forms of the protein, if applicable. The nucleic acid sequences may be naturally occurring or synthetic.

Still further, the “BAG nucleic acid sequence,” “BAG polynucleotide,” and “BAG gene product” refer to nucleic acids provided herein, homologs thereof, and sequences having substantial similarity and function of any of the BAG-family members, respectively. The term “substantially identical”, when used to define either a BAG amino acid sequence or BAG polynucleotide sequence, means that a particular subject sequence, for example, a mutant sequence, varies from the sequence of natural BAG, respectively, by one or more substitutions, deletions, or additions, the net effect of which is to retain at least some of the biological activity found in the native BAG protein, respectively. Alternatively, DNA analog sequences are “substantially identical” to specific DNA sequences disclosed herein if: (a) the DNA analog sequence is derived from coding regions of the natural BAG gene, respectively; or (b) the DNA analog sequence is capable of hybridization to DNA sequences of BAG under moderately stringent conditions and BAG, respectively having biological activity similar to the native proteins; or (c) DNA sequences that are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s)” or “moderately stringent conditions”.

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium or moderate stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Naturally, the present invention also encompasses nucleic acid sequences that are complementary, or essentially complementary, to the sequences set forth herein, for example, in SEQ.ID.NO. 4, SEQ.ID.NO. 5, SEQ.ID.NO.6, SEQ.ID.NO.9, SEQ.ID.NO.10, SEQ.ID.NO.12, SEQ.ID.NO.14, SEQ.ID.NO.16, SEQ.ID.NO.18, SEQ.ID.NO.20, and SEQ.ID.NO.22. Nucleic acid sequences that are “complementary” are those that are capable of base-pairing according to the standard Watson-Crick complementarity rules. As used herein, the terms “complementary sequences” and “essentially complementary sequences” means nucleic acid sequences that are substantially complementary to, as may be assessed by the same nucleotide comparison set forth above, or are able to hybridize to a nucleic acid segment of one or more sequences set forth herein. Such sequences may encode an entire BAG molecule or functional or non-functional fragments or variants thereof.

A. Mutagenesis

In some embodiments of the invention a mutant BAG nucleic acid is utilized. Where employed, mutagenesis of BAG nucleic acid sequences will be accomplished by a variety of standard, mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

Random mutagenesis also may be introduced using error prone PCR (Cadwell and Joyce, 1992). The rate of mutagenesis may be increased by performing PCR in multiple tubes with dilutions of templates.

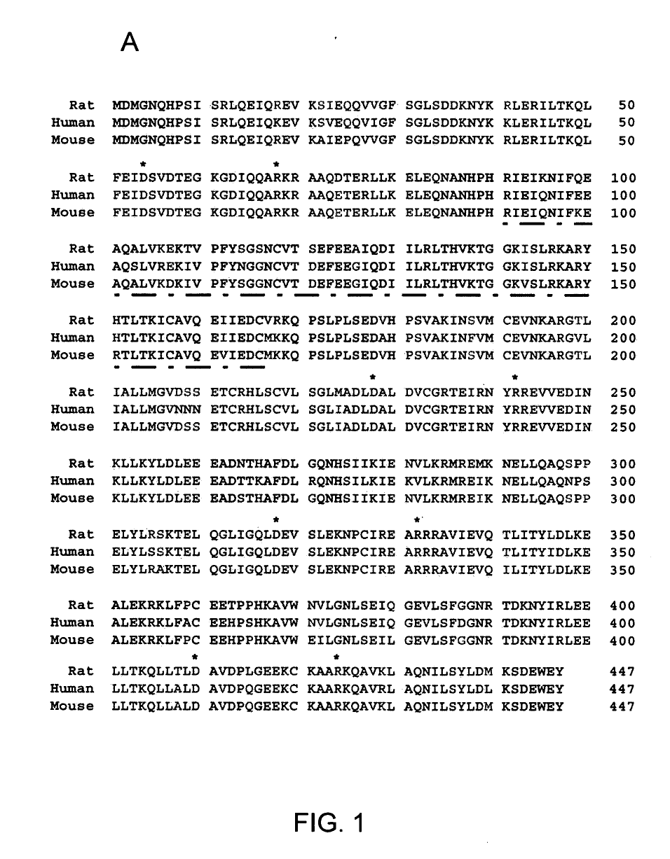

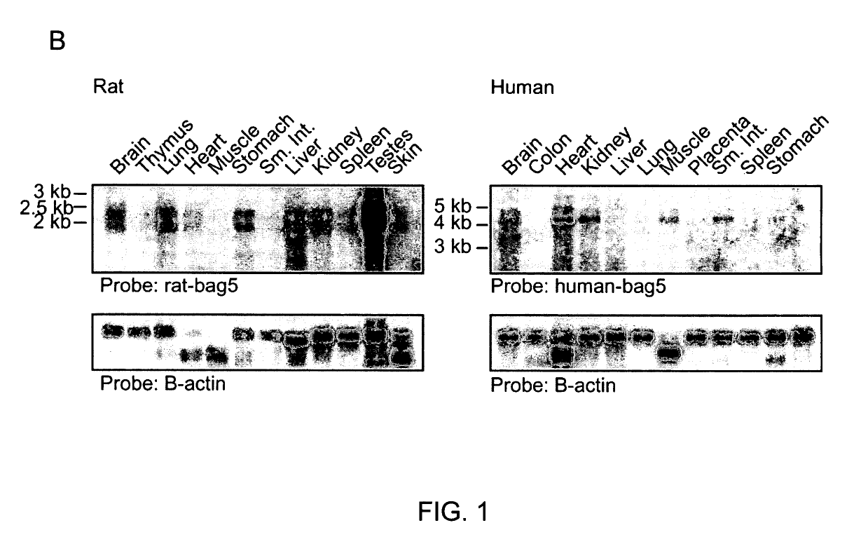

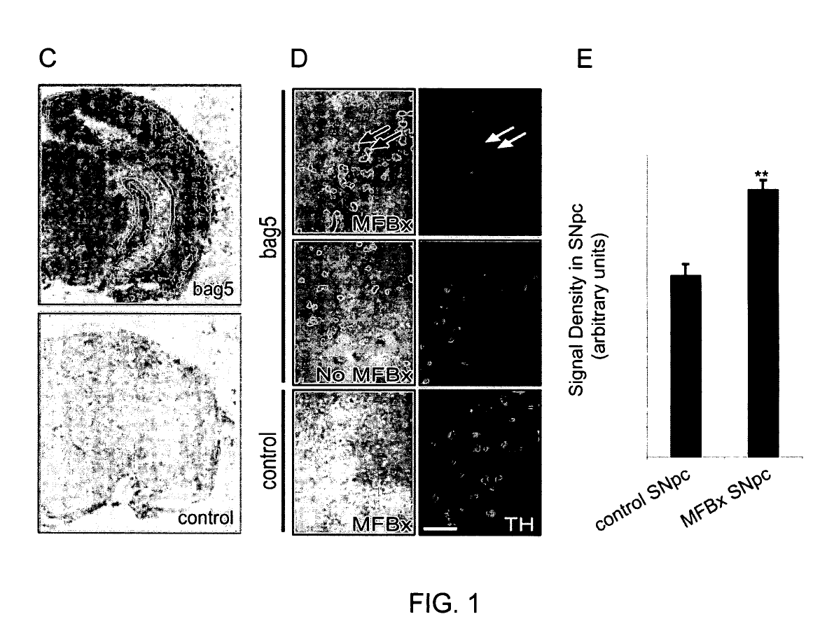

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989). This type of mutagenesis technique can be used to produce a mutant nucleic acid molecule of a BAG-family molecule that encodes a mutant BAG polypeptide, such as BAG5, for example, SEQ.ID.NO.8, also shown in FIG. 1A are the BAG5 nucleic acid sequences form rat, mouse and human. The asterisk indicates the residues that were substituted with alanine to produce a mutant nucleic acid molecule.

Another type of mutagenesis technique that can be employed is in vitro scanning saturation mutagenesis which provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

Yet further, structure-guided site-specific mutagenesis represents a powerful tool for the dissection and engineering of protein-ligand interactions (Wells, 1996, Braisted et al., 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

B. Expression Vectors

The present invention may involve using expression constructs as the pharmaceutical composition. In certain embodiments, it is contemplated that the expression construct comprises polynucleotide sequences encoding polypeptides which can act as inhibitors of BAG and/or BAG-related compounds or related-compounds, for example the expression construct may comprise a nucleic acid sequence encoding an antisense molecule or an shRNA molecule (e.g., SEQ.ID.NO. 7), or a nucleic acid sequence encoding a mutant nucleic acid molecule (e.g., SEQ.ID.NO.9), or a mutant nucleic acid sequence encoding a mutant polypeptide (e.g., SEQ.ID.NO. 8).

In certain embodiments, the present invention involves the manipulation of genetic material to produce expression constructs that encode inhibitors of BAG and/or BAG-related compounds, more specifically BAG5. Thus, the BAG inhibitor and/or related-compound is contained in an expression vector. Such methods involve the generation of expression constructs containing, for example, a heterologous nucleic acid sequence encoding an inhibitor of interest and a means for its expression, replicating the vector in an appropriate cell, obtaining viral particles produced therefrom, and infecting cells with the recombinant virus particles.

As used in the present invention, the term “expression vector” refers to any type of genetic construct comprising a nucleic acid coding for a BAG inhibitor and/or related compounds. In some cases, DNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra. It is contemplated in the present invention, that virtually any type of vector may be employed in any known or later discovered method to deliver nucleic acids encoding an inhibitor of BAG or related molecules. Where incorporation into an expression vector is desired, the nucleic acid encoding an BAG inhibitor or related molecule may also comprise a natural intron or an intron derived from another gene. Such vectors may be viral or non-viral vectors as described herein, and as known to those skilled in the art. An expression vector comprising a nucleic acid encoding an BAG inhibitor or related molecule may comprise a virus or engineered construct derived from a viral genome.

In particular embodiments of the invention, a plasmid vector is contemplated for use to transform a host cell. In general, plasmid vectors containing replicon and control sequences that are derived from species compatible with the host cell are used in connection with these hosts. The vector ordinarily carries a replication site, as well as marking sequences that are capable of providing phenotypic selection in transformed cells. Plasmid vectors are well known and are commercially available. Such vectors include, but are not limited to, the commercially available pSupervector (OligoEngine, Seattle, Wash.), pSuppressorNeo vector (IMGENEX Corporation) and pSilencer™ siRNA expression vectors (Ambion, Austin Tex.). Other vectors that may be employed in the present invention include, but are not limited to, the following exemplary eukaryotic vectors: pWLNEO, pSV2CAT, pOG44, PXT1, pSG (Stratagene) pSVK3, pBSK, pBR322, pUC vectors, vectors that contain markers that can be selected in mammalian cells, such as pcDNA3.1, episomally replicating vectors, such as the pREP series of vectors, pBPV, pMSG, pSVL (Pharmacia), adenovirus vector (AAV; pCWRSV, Chatterjee et al. (1992)); retroviral vectors, such as the pBABE vector series, a retroviral vector derived from MoMuLV (pGINa, Zhou et al., (1994)); and pTZ18U (BioRad, Hercules, Calif.).

In one embodiment, a gene encoding a BAG inhibitor or structural/functional domain thereof or a BAG-related compound is introduced in vivo in a viral vector. The ability of certain viruses to enter cells via receptor-mediated endocytosis and to integrate into the host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Such vectors include an attenuated or defective DNA virus, such as but not limited to herpes simplex virus (HSV), papilloma virus, Epstein Barr virus (EBV), adenovirus, adeno-associated virus (AAV), lentivirus and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, any tissue can be specifically targeted. Examples of particular vectors include, but are not limited to, a defective herpes virus 1 (HSV1) vector (Kaplitt et al., 1991) an attenuated adenovirus vector, (Stratford-Perricaudet et al., 1992), and a defective adeno-associated virus vector (Samulski et al., 1987 and Samulski et al., 1989). Such vectors may be used to (i) transform cell lines in vitro for the purpose of expressing the BAG molecules or inhibitors thereof, such as antisense or RNAi molecules (e.g., siRNA molecules or shRNA) (ii) to transform cells in vitro or in vivo to provide therapeutic molecules for gene therapy. Thus, the present invention contemplates viral vectors such as, but not limited to, an adenoviral vector, an adeno-associated viral vector, a retroviral vector, a lentiviral vector, a herpes viral vector, polyoma viral vector or hepatitis B viral vector.

Preferably, for in vitro administration, an appropriate immunosuppressive treatment is employed in conjunction with the viral vector, e.g., adenovirus vector, to avoid immunodeactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors (Wilson, Nature Medicine (1995). In addition, it is advantageous to employ a viral vector that is engineered to express a minimal number of antigens.

In another embodiment the gene can be introduced in a retroviral vector, e.g., as described in U.S. Pat. No. 5,399,346; Mann et al., 1983; U.S. Pat. No. 4,650,764; U.S. Pat. No. 4,980,289; Markowitz et al., 1988; U.S. Pat. No. 5,124,263; International Patent Publication No. WO 95/07358; and Kuo et al., 1993, each of which is incorporated herein by reference in its entirety. Targeted gene delivery is described in International Patent Publication WO 95/28494.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes. The use of lipofection to introduce exogenous genes into the specific organs in vivo has certain practical advantages. Molecular targeting of liposomes to specific cells represents one area of benefit. Lipids may be chemically coupled to other molecules for the purpose of targeting. Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (Wu and Wu, 1988).

As used herein, the terms “cell,” “cell line,” and “cell culture” may be used interchangeably. All of these terms also include their progeny, which is any and all subsequent generations formed by cell division. It is understood that all progeny may not be identical due to deliberate or inadvertent mutations. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, BAG molecule or antisense or siRNA or shRNA or a construct thereof. Therefore, recombinant cells are distinguishable from naturally occurring cells that do not contain a recombinantly introduced nucleic acid.

In certain embodiments, it is contemplated that nucleic acid or proteinaceous sequences may be co-expressed with other selected nucleic acid or proteinaceous sequences in the same host cell. Co-expression may be achieved by co-transfecting the host cell with two or more distinct recombinant vectors. Alternatively, a single recombinant vector may be constructed to include multiple distinct coding regions for nucleic acids, which could then be expressed in host cells transfected with the single vector.

A gene therapy vector as described above can employ a transcription control sequence operably associated with the sequence for the BAG inhibitor or related compound inserted in the vector. Such an expression vector is particularly useful to regulate expression of a therapeutic BAG inhibitor.

C. Transcription Factors and Nuclear Binding Sites

Transcription factors are regulatory proteins that binds to a specific DNA sequence (e.g., promoters and enhancers) and regulate transcription of an encoding DNA region. Typically, a transcription factor comprises a binding domain that binds to DNA (a DNA binding domain) and a regulatory domain that controls transcription. Where a regulatory domain activates transcription, that regulatory domain is designated an activation domain. Where that regulatory domain inhibits transcription, that regulatory domain is designated a repression domain.

Activation domains, and more recently repression domains, have been demonstrated to function as independent, modular components of transcription factors. Activation domains are not typified by a single consensus sequence but instead fall into several discrete classes: for example, acidic domains in GAL4 (Ma et al., 1987), GCN4 (Hope et al., 1987), VP16 (Sadowski et al., 1988), and GATA-1 (Martin, et al. 1990); glutamine-rich stretches in Sp1 (Courey et al., 1988) and Oct-2/OTF2 (Muller-Immergluck et al., 1990; Gerster et al., 1990); proline-rich sequences in CTF/NF-1 (Mermod et al., 1989); and serine/threonine-rich regions in Pit-1/GH-F-1 (Theill et al., 1989) all function to activate transcription. The activation domains of fos and jun are rich in both acidic and proline residues (Abate et al, 1991; Bohmann et al., 1989); for other activators, like the CCAAT/enhancer-binding protein C/EBP (Friedman et al., 1990), no evident sequence motif has emerged. Still further, other activation domains may include heat shock factor 1 (hsf1) (Hong et al., FEBS Lett. 2004 Feb. 13;559:165-70; Xie et al., J Biol. Chem. 2003; 278:4687-98).

Thus, in the present invention, it is contemplated that transcription factors can be used to inhibit the expression of BAG.

D. Antisense and Ribozymes

An antisense molecule that binds to a translational or transcriptional start site, or splice junctions, are ideal inhibitors. Antisense, ribozyme, and double-stranded RNA molecules target a particular sequence to achieve a reduction or elimination of a particular polypeptide, such as BAG, more particularly BAG5. Thus, it is contemplated that antisense, ribozyme, and double-stranded RNA, and RNA interference molecules are constructed and used to inhibit BAG expression.

1. Antisense Molecules

Antisense methodology takes advantage of the fact that nucleic acids tend to pair with complementary sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, are employed to inhibit gene transcription or translation or both within a host cell, either in vitro or in vivo, such as within a host animal, including a human subject.

Antisense constructs are designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction are used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

It is advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

2. RNA Interference

It is also contemplated in the present invention that double-stranded RNA is used as an interference molecule, e.g., RNA interference (RNAi). RNA interference is used to “knock down” or inhibit a particular gene of interest by simply injecting, bathing or feeding to the organism of interest the double-stranded RNA molecule. This technique selectively “knock downs” gene function without requiring transfection or recombinant techniques (Giet, 2001; Hammond, 2001; Stein P, et al., 2002; Svoboda P, et al., 2001; Svoboda P, et al., 2000).

Another type of RNAi is often referred to as small interfering RNA (siRNA), which may also be utilized to inhibit BAG. A siRNA may comprises a double stranded structure or a single stranded structure, the sequence of which is “substantially identical” to at least a portion of the target gene (See WO 04/046320, which is incorporated herein by reference in its entirety). “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides between such sequences. Identity can be readily calculated. See, for example: Computational Molecular Biology, Lesk, A. M., ed. Oxford University Press, New York, 1988; Biocomputing: Informatics and Genome Projects, Smith, D. W., ea., Academic Press, New York, 1993, and the methods disclosed in WO 99/32619, WO 01/68836, WO 00/44914, and WO 01/36646, specifically incorporated herein by reference. While a number of methods exist for measuring identity between two nucleotide sequences, the term is well known in the art. Methods for determining identity are typically designed to produce the greatest degree of matching of nucleotide sequence and are also typically embodied in computer programs. Such programs are readily available to those in the relevant art. For example, the GCG program package (Devereux et al.), BLASTP, BLASTN, and FASTA (Atschul et al.) and CLUSTAL (Higgins et al., 1992; Thompson, et al., 1994).

Thus, siRNA contains a nucleotide sequence that is essentially identical to at least a portion of the target gene, BAG. More particularly, the siRNA molecule is a BAG5 siRNA molecule that contains a nucleotide sequence that is essentially identical to at least a portion of SEQ.ID.NO. 4, SEQ.ID.NO. 5, SEQ.ID.NO.6, SEQ.ID.NO.9, SEQ.ID.NO.10, SEQ.ID.NO.12, SEQ.ID.NO.14, SEQ.ID.NO.16, SEQ.ID.NO.18, SEQ.ID.NO.20, and/or SEQ.ID.NO.22. Preferably, the siRNA contains a nucleotide sequence that is completely identical to at least a portion of the target gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. It is preferred that there be 100% sequence identity between the dsRNA for use as siRNA and at least 15 contiguous nucleotides of the target gene (e.g., BAG1, BAG2, BAG3, BAG4, and BAG5), although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

There are several methods for preparing siRNA, such as chemical synthesis, in vitro transcription, siRNA expression vectors, and PCR expression cassettes. For example, siRNAs can be constructed in vitro using DNA oligonucleotides. These oligonucleotides can be constructed to include an 8 base sequence complementary to the 5′ end of the T7 promoter primer included in the Silencer siRNA (Ambion Construction Kit 1620). Each gene specific oligonucleotide is annealed to a supplied T7 promoter primer, and a fill-in reaction with Klenow fragment generates a full-length DNA template for transcription into RNA. Two in vitro transcribed RNAs (one the antisense to the other) are generated by in vitro transcription reactions and then hybridized to each other to make double-stranded RNA. The double-stranded RNA product is treated with DNase (to remove the DNA transcription templates) and RNase (to polish the ends of the double-stranded RNA), and column purified to provide the siRNA that can be delivered and tested in cells.

Irrespective of which method one uses, the first step in designing an siRNA molecule is to choose the siRNA target site, which can be any site in the target gene. In certain embodiments, one of skill in the art may manually select the target selecting region of the gene, which may be an ORF (open reading frame) as the target selecting region and may preferably be 50-100 nucleotides downstream of the “ATG” start codon. However, there are several readily available programs available to assist with the design of siRNA molecules, for example siRNA Target Designer by Promega, siRNA Target Finder by GenScript Corp., siRNA Retriever Program by Imgenex Corp., EMBOSS siRNA algorithm, siRNA program by Qiagen, Ambion siRNA predictor, Ambion siRNA predictor, Whitehead siRNA prediction, and Sfold. Thus, it is envisioned that any of the above programs may be utilized to produce siRNA molecules that can be used in the present invention.

Construction of siRNA vectors that express siRNAs within mammalian cells typically use an RNA polymerase III promoter to drive expression of a short hairpin RNA (shRNA) that mimics the structure of an siRNA. The insert that encodes this hairpin is designed to have two inverted repeats separated by a short spacer sequence. One inverted repeat is complementary to the mRNA to which the siRNA is targeted. A string of six consecutive thymidines added to the 3′ end serves as a pol III transcription termination site. Once inside the cell, the vector constitutively expresses the hairpin RNA. The hairpin RNA is processed into an siRNA which induces silencing of the expression of the target gene, which is called RNA interference (RNAi).

In most siRNA expression vectors described to date, one of three different RNA polymerase III (pol III) promoters is used to drive the expression of a small hairpin siRNA (See US Application No. US20040220132, which is incorporated herein by reference in its entirety). These promoters include the well-characterized human and mouse U6 promoters and the human H1 promoter. RNA pol III was chosen to drive siRNA expression because it expresses relatively large amounts of small RNAs in mammalian cells and it terminates transcription upon incorporating a string of 3-6 uridines.

3. Ribozymes

Ribozymes are RNA-protein complexes that cleave nucleic acids in a site-specific fashion. Ribozymes have specific catalytic domains that possess endonuclease activity (Kim and Cech, 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression is particularly suited to therapeutic applications (Scanlon et al., 1991; Sarver et al., 1990; Sioud et al., 1992). Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis δ virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in BAG targeted ribozymes is simply a matter of preparing and testing a given sequence, and is a routinely practiced screening method known to those of skill in the art.

E. Protein Variants

Amino acid sequence variants of the BAG-family proteins can be used as inhibitors of BAG activity, for example SEQ.ID.NO.8. These variants can be substitutional, insertional or deletion variants. These variants may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

Substitutional variants or replacement variants typically contain the exchange of one amino acid for another at one or more sites within the protein. Substitutions can be conservative, that is, one amino acid is replaced with one of similar shape and charge. Conservative substitutions are well known in the art and include, for example, the following: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtains a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

1. Fusion Proteins

A specialized kind of insertional variant is the fusion protein. This molecule generally has all or a substantial portion of the native molecule, linked at the N- or C-terminus, to all or a portion of a second polypeptide. For example, a fusion protein of the present invention can includes the addition of a protein transduction domains, for example, but not limited to Antennepedia transduction domain (ANTP), HSV1 (VP22) and HIV-1(Tat). Fusion proteins containing protein transduction domains (PTDs) can traverse biological membranes efficiently, thus delivering the protein of interest into the cell. This type of fusion protein is part of the transport system that is mediated by cell-penetrating peptide, which consist of short peptide sequences that rapidly translocate large molecules into the cell interior in a seemingly energy- and receptor-independent manner (See, Jarver P, Langel U, Drug Discov Today. 2004 May 1;9(9):395-402)).

Yet further, inclusion of a cleavage site at or near the fusion junction will facilitate removal of the extraneous polypeptide after purification. Other useful fusions include linking of functional domains, such as active sites from enzymes, glycosylation domains, other cellular targeting signals or transmembrane regions.

2. Domain Switching

An interesting series of variants can be created by substituting homologous regions of various proteins. This is known, in certain contexts, as “domain switching.”

Domain switching involves the generation of chimeric molecules using different but, in this case, related polypeptides. By comparing various BAG proteins, one can make predictions as to the functionally significant regions of these molecules. It is possible, then, to switch related domains of these molecules in an effort to determine the criticality of these regions to function of the protein. These molecules may have additional value in that these “chimeras” can be distinguished from natural molecules, while possibly providing the same function.

3. Synthetic Peptides

The present invention also describes smaller BAG-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

F. Antigen Compositions

Other inhibitors of BAG-related proteins can be antibody compositions. Thus, present invention also provides for the use of BAG proteins or polypeptides as antigens for the immunization of animals relating to the production of antibodies, which can further be used as BAG inhibitors. It is envisioned that BAG proteins, polypeptides or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyanin (KLH) or bovine serum albumin (BSA).

1. Antibody Production

In certain embodiments, the present invention provides antibodies that bind with high specificity to the BAG proteins or polypeptides provided herein. Thus, antibodies that bind to the a BAG-family protein, such as the ones described in U.S. Pat. No. 6,696,558, which is incorporated herein by reference, are provide. More specifically, SEQ.ID.NO:1, SEQ.ID.NO:2, SEQ.ID.NO:3, SEQ.ID.NO.8, SEQ.ID.NO.11, SEQ.ID.NO.13, SEQ.ID.NO.15, SEQ.ID.NO.17, SEQ.ID.NO.19, SEQ.ID.NO.21, and/or SEQ.ID.NO.23 are provided. In addition to antibodies generated against the full length proteins, antibodies may also be generated in response to smaller constructs comprising epitopic core regions, including wild-type and mutant epitopes. Monoclonal antibodies and/or polyclonal antibodies can be generated using techniques that are well known and used in the art. MAbs may be readily prepared through use of well-known techniques, such as those exemplified in U.S. Pat. No. 4,196,265, incorporated herein by reference.

Humanized antibodies are also contemplated, as are chimeric antibodies from mouse, rat, or other species, bearing human constant and/or variable region domains, bispecific antibodies, recombinant and engineered antibodies and fragments thereof.

2. Antibody Conjugates

The present invention further provides antibodies against BAG, generally of the monoclonal type, that are linked to one or more other agents to form an antibody conjugate. Any antibody of sufficient selectivity, specificity and affinity may be employed as the basis for an antibody conjugate. Such properties may be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected, and further quantified if desired. Another such example is the formation of a conjugate comprising an antibody linked to a cytotoxic or anti-cellular agent, as may be termed “immunotoxins” (described in U.S. Pat. Nos. 5,686,072, 5,578,706, 4,792,447, 5,045,451, 4,664,911 and 5,767,072, each incorporated herein by reference).

Still further, the antibody can be conjugated to a molecule such that the conjugated antibody can be delivered into the brain by receptor-mediated transcytosis through the blood-brain barrier. Suitable transportable molecules that can be conjugated to the antibody include, for example, histone, insulin, transferrin, insulin-like growth factor I (IGF-1), insulin-like growth factor II (IGF-II), basic albumin and prolactin. The use of transportable molecules is further described in U.S. Pat. No. 4,902,505, which is incorporated herein by reference. Another example of a protein that can be conjugated to the antibody for high-capacity transport across the blood-brain barrier is receptor-associated protein (RAP) (Pan et al., J Cell Sci., 2004, 117:5071-8).

IV. Screening for Inhibitors

The present invention comprises methods for identifying inhibitors that affect the activity and/or expression of a BAG-family protein and/or gene. These assays may comprise random screening of large libraries of candidate substances; alternatively, the assays may be used to focus on particular classes of compounds selected with an eye towards structural attributes that are believed to make them more likely to modulate the function or activity or expression of a BAG-family protein or gene. Yet further, the present invention encompasses inhibitors identified in U.S. Pat. No. 6,596,527, which is incorporated herein by reference in its entirety.

By function, it is meant that one may assay for mRNA expression, protein expression, protein activity, binding activity, or ability to associate and/or dissociate from other members of the complex and otherwise determine functions contingent on the BAG proteins or nucleic acid molecules.

A. Inhibitors

The present invention further comprises methods for identifying, making, generating, providing, manufacturing or obtaining inhibitors of BAG activity or expression. BAG nucleic acid sequences or polypeptide sequences may be used as a target in identifying compounds that inhibit cell loss (e.g., neuronal cell loss and/or neuronal degeneration, specifically dopaminergic degeneration), and/or enhance cell survival. In further embodiments the inhibitors of BAG activity or expression enhance Hsp70 chaperone activity, such as enhance Hsp 70-mediated refolding of misfolded proteins. Still further, the inhibitors of BAG activity or expression (e.g., BAG5) enhance parkin E3 ubiquitin-ligase activity or inhibit parkin sequestration and protein aggregation. These assays may comprise random screening of large libraries of candidate substances; alternatively, the assays may be used to focus on particular classes of compounds selected with an eye towards structural attributes that are believed to make them more likely to inhibit the function of BAG molecules. By function, it is meant that one may assay for inhibition of activity of BAG in cells, or inhibition of expression of BAG. Such assays may include for example luciferase reporter system in which luciferase activity is measured. Other assays may include generation of stable cell lines and the use of tranasgeneic animals.

To identify, make, generate, provide, manufacture or obtain a BAG inhibitor, one generally will determine the activity of the BAG molecule in the presence, absence, or both of the candidate substance, wherein an inhibitor is defined as any substance that down-regulates, reduces, inhibits or decreases BAG expression or activity. For example, a method may generally comprise: providing a candidate substance suspected of decreasing BAG expression or activity; assessing the ability of the candidate substance to decrease BAG expression or activity; selecting an BAG inhibitor; and manufacturing the inhibitor.

In further embodiments, a BAG polypeptide or nucleic acid may be provided in a cell or a cell free system and the BAG polypeptide or nucleic acid may be contacted with the candidate substance. Next, an inhibitor is selected by assessing the effect of the candidate substance on BAG activity or BAG expression. Upon identification of the inhibitor, the method may further provide manufacturing of the inhibitor.

As used herein, the term “candidate substance” refers to any molecule that may potentially inhibit BAG activity, expression or function. Candidate compounds may include fragments or parts of naturally-occurring compounds or may be found as active combinations of known compounds that are otherwise inactive. The candidate substance can be a nucleic acid (e.g., antisense molecule, siRNA molecule or shRNA molecules), a polypeptide (e.g., antibodies), a small molecule, etc. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

In specific embodiments, a small molecule library that is created by chemical genetics may be screened to identify a candidate substance that may be a modulator of the present invention (Clemons et al., 2001; Blackwell et al., 2001). Chemical genetics is the technology that uses small molecules to modulate the functions of proteins rapidly and conditionally. The basic approach requires identification of compounds that regulate pathways and bind to proteins with high specificity. Small molecules are prepared using diversity-oriented synthesis, and the split-pool strategy to allow spatial segregation on individual polymer beads. Each bead contains compounds to generate a stock solution that can be used for many biological assays.

The most useful pharmacological compounds may be compounds that are structurally related to compounds which interact naturally with compounds that modulate BAG transcription or activity. Creating and examining the action of such molecules is known as “rational drug design,” and include making predictions relating to the structure of target molecules. Thus, it is understood that the candidate substance identified by the present invention may be a small molecule activator or any other compound (e.g., polypeptide or polynucleotide) that may be designed through rational drug design starting from known inhibitors of BAG.

The goal of rational drug design is to produce structural analogs of biologically active target compounds. By creating such analogs, it is possible to fashion drugs which are more active or stable than the natural molecules, which have different susceptibility to alteration or which may affect the function of various other molecules. In one approach, one would generate a three-dimensional structure for a molecule similar to BAG, and then design a molecule for its ability to interact with an BAG-related molecule. This could be accomplished by X-ray crystallography, computer modeling or by a combination of both approaches. The same approach may be applied to identifying interacting molecules of BAG.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

It will, of course, be understood that all the screening methods of the present invention are useful in themselves notwithstanding the fact that effective candidates may not be found. The invention provides methods for screening for such candidates, not solely methods of finding them.

B. In Vitro Assays

A quick, inexpensive and easy assay to run is a binding assay. Binding of a molecule to a target (e.g., BAG1, BAG2, BAG3, BAG4, BAG5, BAG6) may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to BAG molecules or fragments thereof are provided.

A target BAG protein may be either free in solution, fixed to a support, expressed in or on the surface of a cell. Either the BAG protein or the compound may be labeled, thereby indicating if binding has occurred. In another embodiment, the assay may measure the activation of BAG to a natural or artificial substrate or binding partner. Competitive binding assays can be performed in which one of the agents is labeled. Usually, the target BAG protein will be the labeled species, decreasing the chance that the labeling will interfere with the binding moiety's function. One may measure the amount of free label versus bound label to determine binding or activation of binding. These approaches may be utilized on BAG molecules.

A technique for high throughput screening of compounds is described in WO 84/03564. Large numbers of small peptide test compounds are synthesized on a solid substrate, such as plastic pins or some other surface. The peptide test compounds are reacted with, for example, BAG protein and washed. Bound polypeptide is detected by various methods.

C. In Cyto Assays

Various cell lines that express BAG proteins can be utilized for screening of candidate substances. For example, cells containing BAG proteins with an engineered indicator can be used to study various functional attributes of candidate compounds. In such assays, the compound would be formulated appropriately, given its biochemical nature, and contacted with a target cell. This same approach may utilized to study various functional attributes of candidate compounds that effect BAG.

Depending on the assay, culture may be required. As discussed above, the cell may then be examined by virtue of a number of different physiologic assays (e.g., growth, size, or survival). Alternatively, molecular analysis may be performed in which the function of BAG and BAG related pathways may be explored. This involves assays such as those for protein production, enzyme function, substrate utilization, mRNA expression (including differential display of whole cell or polyA RNA) and others.

D. In Vivo Assays

The present invention particularly contemplates the use of various animal models. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), and U.S. Pat. No. 6,201,165, the teachings of which are generally known and are incorporated herein.

Treatment of animals with test compounds (e.g., inhibitors of BAG, more specifically, inhibitors BAG5) involve the administration of the compound, in an appropriate form, to the animal. Administration is by any route that could be utilized for clinical or non-clinical purposes, including but not limited to oral, nasal, buccal, or even topical. Alternatively, administration may be by intratracheal instillation, bronchial instillation, intradermal, intracranial, intrathecal, subcutaneous, intramuscular, intraperitoneal or intravenous injection. Specifically contemplated are intracranial, and systemic intravenous injection.

E. Production of Inhibitors

In an extension of any of the previously described screening assays, the present invention also provide for methods of producing inhibitors of BAG. The methods comprising any of the preceding screening steps followed by an additional step of “producing or manufacturing the candidate substance identified as an inhibitor of” the screened activity.

V. Methods of Treatment Using at Least One BAG Inhibitor

In certain aspects of the present invention, BAG inhibitors or related-compounds thereof are administered to cells to, inhibit or attenuate cell loss. Such diseases in which cell loss occurs include, but are not limited to eye diseases and liver diseases, as well as neurological conditions and/or diseases, such as neurodegenerative diseases. Other disease or conditions can include, systemic amyloidosis and retinal dystrophies, such as (e.g. retinitis pigmentosa (RP)), for example.

Thus, the present invention pertains to the use of BAG inhibitors for any disease or conditon in which there is an increase in protein aggregation and/or a loss in chaperone activity (Hsp70 or Hsc70), in specific embodiments of the invention. The BAG inhibitors of the present invention are protective of cell death or loss, and thus the inhibitors increase or enhance cell survival.

In certain embodiments, the inhibitors are used to attenuate or inhibit neuronal cell loss. Neuronal cell loss can be due to any event that results in neuronal loss, such as genetic predisposition, congenital dysfunction, apoptosis, ischemic event, immune-mediated, free-radical induced, chemical induced, or any injury that results in a loss of neuronal cells, as well as a progressive loss of neuronal cells. Neurodegeneration typically refers to a progressive loss of neurons or apoptosis of neurons or a progressive loss of neuronal function. Thus, the inhibitors of the present invention are neuroprotective in that they inhibit or attenuate neuronal cell loss and/or neurodegeneration resulting in an increase in survival or function of neuronal cells.

With the administration of an BAG inhibitor, cell loss or cell degeneration is abrogated, slowed, reduced or inhibited due to the decrease in BAG transcription and/or activity. In certain embodiments, the use of a BAG5 inhibitor results in an increase in Hsp70 chaperone activity or an increase in Hsp-70 mediated refolding of misfolded proteins, decreases the levels or amounts of misfolded proteins, increases parkin E3 ubiquition-ligase activity, or inhibits parkin sequestration and protein aggregation. As one of skill in the art is aware, BAG can be acting or interacting with other molecules in addition to Hsp70.

Cells types that can be used in the present invention include, blood cells, immune cells, muscle cells, nerve or neuronal cells, or gland cells. More specifically, cells can be isolated from the following tissues: ameloblasts, axon, basal cells, blood (e.g., lymphocytes), blood vessel, bone, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, prostate, skin, small intestine, spleen, stem cells, stomach, and/or testes.

In certain embodiments, the cells are neuronal cells in which the BAG inhibitors may be administered. Neuronal cells may include, for example, but are not limited to, cholinergic, adrenergic, noradrenergic, dopaminergic, serotonergic, glutaminergic, GABAergic, and glycinergic. In certain embodiments, the neuronal cell is a dopaminergic cell.

An effective amount of a BAG inhibitor that may be administered to a cell includes a dose of about 0.1 nM to about 1000 mM. More specifically, doses of an BAG inhibitor to be administered are from about 0.1 nM to about 10 μM; about 10 μM to about 50 μM; about 50 μM to about 100 μM; about 100 μM to about 150 μM; about 150 μM to about 200 μM; about 200 μM to about 300 μM; about 300 μM to about 400 μM; about 400 μM to about 500 μM; about 500 μM to about 900 μM; about 900 μM to about 20 mM; about 20 mM to about 40 mM; about 40 mM to about 100 mM; and about 100 mM to about 1000 mM. Of course, all of these amounts are exemplary, and any amount in-between these points is also expected to be of use in the invention. Other amounts may be employed so long as they are effective and have little or no toxic effects.

It is envisioned that the BAG inhibitor or related-compound thereof will inhibit the loss of cells or degeneration of at least one cell by measurably slowing, stopping, or reversing the death rate of the cell or apoptotic rate of the cells or loss of function of the cells or decrease protein aggregation in vitro or in vivo. Desirably, the death rate is slowed by 20%, 30%, 50%, or 70% or more, as determined using a suitable assay for determination of cell death rates and/or cell apoptosis.

Still further, the present invention provides methods for the treatment of a disease or condition that comprises cell loss, such as neuronal cell loss, as well as neurodegenerative diseases, by administering a BAG inhibitor (e.g., siRNA, a mutated nucleic acid molecule and/or a small molecule).

The BAG inhibitor or related-compound thereof can be administered parenterally or alimentary. Parenteral administrations include, but are not limited to intravenously, intradermally, intramuscularly, intraarterially, intrathecally, intracranially, subcutaneous, or intraperitoneally U.S. Application No. US20040220132, U.S. Pat. Nos. 6,613,308, 5,466,468, 5,543,158; 5,641,515; and 5,399,363 (each specifically incorporated herein by reference in its entirety). Alimentary administrations include, but are not limited to orally, buccally, rectally, or sublingually.

Blocking cell loss or enhancing neuroprotection by removing the deleterious consequences of BAG activity can have several applications. This form of therapy can be used in a number of neurological and psychiatric disorders affected by neuronal loss and where there can be clinical benefit from decreasing neuronal loss. For example, targeting the hippocampal neuronal loss can be important in depression, epilepsy, post cranial irradiation, steroid induced impairment in neurogenesis, stress disorders, cognitive disorders and in Alzheimer's disease. Targeting the cortical, striatal, substantia nigra, brainstem and cerebellar loss is important in Huntington's Disease, Alzheimers, multiple system atrophy, Parkinson's disease, post-cranial irradiation disorders, paraneoplastic disorders and the Spinocerebellar ataxias. This method may also be applicable to treat the neuronal loss that occurs as a consequence of a number of disorders including but not limited to congenital disorders, stroke, anoxia, hypoxia, stroke, hypoglycemia, metabolic disorders, head injury, drug and alcohol toxicity, nutritional deficiencies, auto-immune infectious and inflammatory processes including Multiple sclerosis, Guillain-Barre syndrome, chronic idiopathic demyelinating neuropathy (CID), neuropathy, ataxias and myasthenia gravis.

Treatment methods will involve treating an individual with an effective amount of a composition containing an BAG inhibitor or related-compound thereof. An effective amount is described, generally, as that amount sufficient to detectably and repeatedly to ameliorate, reduce, minimize or limit the extent of a disease or its symptoms. More specifically, it is envisioned that the treatment with the inhibitor of BAG or related-compounds thereof will decrease or attenuate cell death or apoptosis, enhance Hsp70 chaperone activity, enhance parkin E3 ubiquitin-ligase activity, increase the refolding of misfolded proteins, reduce parkin sequestration and protein aggregation.

The effective amount or “therapeutically effective amounts” of the inhibitor of BAG or related-compounds thereof to be used are those amounts effective to produce beneficial results, particularly with respect to cancer treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as an BAG inhibitor or related-compounds thereof for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

A therapeutically effective amount of a BAG inhibitor or related-compounds thereof as a treatment varies depending upon the host treated and the particular mode of administration. In one embodiment of the invention the dose range of the BAG inhibitor or related-compounds thereof will be about 0.5 mg/kg body weight to about 500 mg/kg body weight. The term “body weight” is applicable when an animal is being treated. When isolated cells are being treated, “body weight” as used herein should read to mean “total cell body weight”. The term “total body weight” may be used to apply to both isolated cell and animal treatment. All concentrations and treatment levels are expressed as “body weight” or simply “kg” in this application are also considered to cover the analogous “total cell body weight” and “total body weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for an BAG inhibitor or related-compounds thereof.

Administration of the therapeutic BAG inhibitor composition of the present invention to a patient or subject will follow general protocols for the administration of pharmaceuticals, taking into account the toxicity, if any, of the BAG inhibitor. It is expected that the treatment cycles would be repeated as necessary. It also is contemplated that various standard therapies, may be applied in combination with the described therapy.

The treatments may include various “unit doses.” Unit dose is defined as containing a predetermined quantity of the therapeutic composition (an BAG inhibitor or its related-compounds thereof) calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time.

Suitable dose ranges for intracranial administration of a viral expression vector containing a BAG inhibitor are generally about 10³ to 10¹⁵ infectious units of viral vector per microliter delivered in 1 to 3000 microliters of single injection volume. Addition amounts of infections units of vector per micro liter would generally contain about 10⁴, 10⁵, 10⁶, 10⁷, 10⁸, 10⁹, 10¹⁰, 10¹¹, 10¹², 10¹³, 10¹⁴ infectious units of viral vector delivered in about 10, 50, 100, 200, 500, 1000, or 2000 microliters. Effective doses may be extrapolated from dose-responsive curves derived from in vitro or in vivo test systems.

In further aspects, an effective amount of an BAG inhibitor or related-compound (e.g., BAG5 inhibitor) thereof may be administered to a subject suffering from Parkinson's disease (PD) more specifically, sporadic PD. The effectiveness of BAG inhibitor therapy according to the present invention can be determined in the treatment of PD by diagnostic methods that are known and used in the art.

In general, neurological diseases or conditions are diagnosed based upon the accumulation of physical, chemical, and/or historical behavioral data on each patient. One of skill in the art is able to perform the appropriate examinations to accumulate such data. One type of examination can include neurological examinations, which can include mental status evaluations, which can further include a psychiatric assessment. Other types of examinations can include, but are not limited to, motor examination, cranial nerve examination, and neuropsychological tests (e.g., Minnesota Multiphasic Personality Inventory, Beck Depression Inventory, or Hamilton Rating Scale for Depression). Thus, any improvement is based upon an improvement in any of the examinations that are used to diagnose a neurological disorder.

In addition to the above examinations, imaging techniques can be used to determine normal and abnormal brain function that can result in disorders. Functional brain imaging allows for localization of specific normal and abnormal functioning of the nervous system. This includes electrical methods such as electroencephalography (EEG), magnetoencephalography (MEG), single photon emission computed tomography (SPECT), (SPECT), as well as metabolic and blood flow studies such as functional magnetic resonance imaging (fMRI), and positron emission tomography (PET), which can be utilized to localize brain function and dysfunction.

In certain embodiments, in order to ascertain if the BAG inhibitors are decreasing neurodegeneration in a subject suffering from Parkinson's, diagnostic techniques such as ¹⁸F-dopa technique can be used to monitor the progression of nigrostriatal dysfunction in subjects. Other presynaptic dopaminergic functions can be measured using ¹¹C-nomifensine (a dopamine reuptake site inhibitor) and ^{99m}Tc-labeled carbomethyoxy-3-beta-(4-idodophenyl)-tropane. Still further, perfusion mapping can be used to indicate the nigrostriatal degeneration.

VI. Combined Therapy with BAG Inhibitors

In the context of the present invention, it is contemplated that the BAG inhibitor or related-compounds thereof may be used in combination with an additional standard agent to more effectively treat the neurodegenerative disease and/or neurological conditions that are associated with neuronal loss.

Standard therapy can include therapies that are administered to treat the neurological symptoms that are associated with the neurodegenerative disease, such symptoms can include, but are not limited to pain, weakness, sensory loss, paresthesias, burning dysesthesias, ataxia, aphasia, dysarthria, dysphagia, and respiratory failure.

Still further, other therapies can include stimulatory or inhibitory drugs or compositions, such as anesthetic agents, synthetic or natural peptides or hormones, neurotransmitters, cytokines, antibodies, and other intracellular and intercellular chemical signals and messengers, and the like. In addition, certain neurotransmitters, hormones, and other drugs are excitatory for some tissues, yet are inhibitory to other tissues. Therefore, where, herein, a drug is referred to as an “excitatory” drug, this means that the drug is acting in an excitatory manner, although it may act in an inhibitory manner in other circumstances and/or locations. Similarly, where an “inhibitory” drug is mentioned, this drug is acting in an inhibitory manner, although in other circumstances and/or locations, it may be an “excitatory” drug.

Similarly, excitatory neurotransmitter agonists (e.g., norepinephrine, epinephrine, glutamate, acetylcholine, serotonin, dopamine), agonists thereof, and agents that act to increase levels of an excitatory neurotransmitter(s) (e.g., edrophonium; Mestinon; trazodone; SSRIs (e.g., flouxetine, paroxetine, sertraline, citalopram and fluvoxamine); tricyclic antidepressants (e.g., imipramine, amitriptyline, doxepin, desipramine, trimipramine and nortriptyline), monoamine oxidase inhibitors (e.g., phenelzine, tranylcypromine, isocarboxasid)), generally have an excitatory effect on neural tissue, while inhibitory neurotransmitters (e.g., dopamine, glycine, and gamma-aminobutyric acid (GABA)), agonists thereof, and agents that act to increase levels of an inhibitory neurotransmitter(s) generally have an inhibitory effect. (Dopamine acts as an excitatory neurotransmitter in some locations and circumstances, and as an inhibitory neurotransmitter in other locations and circumstances.) However, antagonists of inhibitory neurotransmitters (e.g., bicuculline) and agents that act to decrease levels of an inhibitory neurotransmitter(s) have been demonstrated to excite neural tissue, leading to increased neural activity. Similarly, excitatory neurotransmitter antagonists (e.g., prazosin, and metoprolol) and agents that decrease levels of excitatory neurotransmitters may inhibit neural activity. Yet further, lithium salts and anesthetics (e.g., lidocane) may also be used in combination with the present invention.

If the neurodegenerative disease that is treated according to the methods of the present invention is Parkinson's Disease, then standard pharmacologic/surgical treatments can be used. Anti-Parkinsonian agents that can be used in combination with the BAG5 inhibitor can include, but are not limited to selegiline, anticholinergics (e.g., benstropine, procyclinde, trihexyphenidyl), amantadine, dopamine agonist (e.g., bromocriptine, pergolide, pramipexole, ropinirole), levodopa (e.g., carbidopa/levodopa, carbidopa/levodopa controlled release), inhibitors of catechol-O-methyltransferase (COMT) (e.g., tolcapone, entacapone), monamine axidase type B (MAO-B) inhibitors, and Nmethyl-D-aspartate (NMDA).

In addition to anti-Parkinsonian agents, surgical treatment can be used in combination with the BAG inhibitors of the present invention. Such surgical treatments can include, but are not limited to thalamotomy, pallidotomy, and deep brain stimulation of the ventral intermediate nucleus (Vim) of the thalamus, subthalamic nucleus (STN), and globus pallidus intemus (GPi), using an implantable pulse generator (IPG).

When an additional therapeutic agent, such as an anti-Parkinsonian agent, is administered, as long as the dose of the additional therapeutic agent does not exceed previously quoted toxicity levels, the effective amounts of the additional therapeutic agent may simply be defined as that amount effective to inhibit and/or reduce cell death or apoptosis or symptoms related to a neurodegenerative disease when administered to an animal in combination with the BAG inhibitor or related-compounds thereof. This may be easily determined by monitoring the animal or patient and measuring those physical and biochemical parameters of health and disease that are indicative of the success of a given treatment. Such methods are routine in animal testing and clinical practice.

To attenuate symptoms related to neurodegenerative diseases or to inhibit or attenuate cell death or apoptosis, using the methods and compositions of the present invention, one would generally contact a cell with BAG inhibitor or related-compounds thereof in combination with an additional therapeutic agent. These compositions would be provided in a combined amount effective to inhibit cell death and/or apoptosis in the cell, or inhibit functional loss of the cell. This process may involve contacting the cells with BAG inhibitor or related-compounds thereof in combination with an additional therapeutic agent or factor(s) at the same time. This may be achieved by contacting the cell with a single composition or pharmacological formulation that includes both agents, or by contacting the cell with two distinct compositions or formulations, at the same time, wherein one composition includes the BAG inhibitor or derivatives thereof and the other includes the additional agent.

Alternatively, treatment with BAG inhibitor or related-compounds thereof may precede or follow the additional agent treatment by intervals ranging from minutes to weeks. In embodiments where the additional agent is applied separately to the cell, one would generally ensure that a significant period of time did not expire between the time of each delivery, such that the agent would still be able to exert an advantageously combined effect on the cell. In such instances, it is contemplated that one would contact the cell with both modalities within about 12-24 hr of each other and, more preferably, within about 6-12 hr of each other, with a delay time of only about 12 hr being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either BAG inhibitor or related-compounds thereof in combination with an additional therapeutic agent such as an anti-Parkinsonian agent will be desired. Various combinations may be employed, where BAG inhibitor or related-compounds thereof is “A” and the additional therapeutic agent is “B”, as exemplified below:

A/B/A B/A/B B/B/A A/A/B B/A/A A/B/B B/B/B/A B/B/A/B

A/A/B/B A/B/A/B A/B/B/A B/B/A/A B/A/B/A B/A/A/B B/B/B/A

A/A/A/B B/A/A/A A/B/A/A A/A/B/A A/B/B/B B/A/B/B B/B/A/B

In addition to pharmaceuticals that can be used in combination with a BAG inhibitor, electrical stimulation may be used to alter the activity of a BAG-family protein, see for instance, U.S. Pat. No. 6,016,449 and U.S. Pat. No. 6,176,242, each of which is incorporated herein by reference in its entirety.

Still further, the present invention can be used with other gene therapies and/or stem cell therapies. It is envisioned that the transplanted cells can replace damaged, degenerating or dead neuronal cells, deliver a biologically active molecule to the predetermined site or to ameliorate a condition and/or to enhance or stimulate existing neuronal cells. Such transplantation methods are described in U.S. Application No. US20040092010, which is incorporated herein by reference in its entirety.

Cells that can be transplanted can be obtained from stem cell lines (i.e., embryonic stem cells, non-embryonic stem cells, etc.) and/or brain biopsies, including tumor biopsies, autopsies and from animal donors. (See U.S. Application No. US20040092010; U.S. Pat. Nos. 5,735,505 and 6,251,669; Temple, Nature Reviews 2:513-520 (2000); Bjorklund and Lindvall, Nat. Neurosci. 3:537-544 (2000)), each of which is incorporated herein by reference in its entirety). Brain stem cells can then be isolated (concentrated) from non-stem cells based on specific “marker” proteins present on their surface. In one such embodiment, a fluorescent antibody specific for such a marker can be used to isolate the stem cells using fluorescent cell sorting (FACS). In another embodiment an antibody affinity column can be employed. Alternatively, distinctive morphological characteristics can be employed.

VII. Formulations and Routes for Administration of BAG Inhibitors or Related-Compounds Thereof

Where clinical applications are contemplated, it will be necessary to prepare pharmaceutical compositions of BAG inhibitors or related-compounds thereof, or any additional therapeutic agent disclosed herein in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The composition(s) of the present invention may be delivered orally, nasally, intramuscularly, intraperitoneally, or intracranially. In some embodiments, local or regional delivery of BAG inhibitors or related-compounds thereof, alone or in combination with an additional therapeutic agent, to a patient with a neurodegenerative condition will be a very efficient method of delivery to counteract the clinical disease. Other examples of delivery of the compounds of the present invention that may be employed include intra-arterial, intravesical, intrathecal, intracranial and intraperitoneal routes.

Intra-arterial administration is achieved using a catheter that is inserted into an artery to an organ or to an extremity. Typically, a pump is attached to the catheter. Because most drugs do not penetrate the blood/brain barrier, intrathecal therapy is used to reach cancer cells in the central nervous system. To do this, drugs are administered directly into the cerebrospinal fluid.

The active compositions of the present invention may include classic pharmaceutical preparations. Administration of these compositions according to the present invention will be via any common route so long as the target tissue is available via that route. This includes but is not limited to, oral, nasal, or buccal routes. Alternatively, administration may be by intradermal, subcutaneous, intramuscular, intraperitoneal or intravenous injection. Such compositions would normally be administered as pharmaceutically acceptable compositions, described supra. The drugs and agents also may be administered parenterally or intraperitoneally. The term “parenteral” is generally used to refer to drugs given intravenously, intramuscularly, or subcutaneously.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to the well known parameters. Suitable excipients for formulation with BAG inhibitors or related-compounds thereof include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

In further embodiments intracranial administration of the BAG inhibitor previously described, may involve the use of devices, systems, and methods for delivery of BAG inhibitors or related compounds to target locations of the brain. The envisioned route of delivery is through the use of implanted, indwelling, intraparenchymal catheters that provide a means for injecting small volumes of fluid directly into local brain tissue. The proximal end of these catheters may be connected to an implanted, intracerebral access port surgically affixed to the patient's cranium, or to an implanted drug pump located in the patient's torso.

Any type of infusion pump can be used in the present invention. For example, “active pumping” devices or so-called peristaltic pumps are described in U.S. Pat. Nos. 4,692,147, 5,840,069, and 6,036,459, which are incorporated herein by reference in their entirety. Peristaltic pumps are used to provide a metered amount of a drug in response to an electronic pulse generated by control circuitry associated within the device. An example of a commercially available peristaltic pump is SynchroMed® implantable pump from Medtronic, Inc., Minneapolis, Minn.

Other pumps that may be used in the present invention include accumulator-type pumps, for example certain external infusion pumps from Minimed, Inc., Northridge, Calif. and Infusaid® implantable pump from Strato/Infusaid, Inc., Norwood, Mass. Passive pumping mechanisms can be used to release an agent in a constant flow or intermittently or in a bolus release. Passive type pumps include, for example, but are not limited to gas-driven pumps described in U.S. Pat. Nos. 3,731,681 and 3,951,147; and drive-spring diaphragm pumps described in U.S. Pat. Nos. 4,772,263, 6,666,845, 6,620,151 all of which are incorporated by reference in their entirety. Pumps of this type are commercially available, for example, Model 3000® from Arrow International, Reading, Penn. and IsoMed® from Medtronic, Inc., Minneapolis, Minn.; AccuRx® pump from Advanced Neuromodulation Systems, Inc., Plano, Tex.

VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Cloning of BAG5 and Vector Construction

Rat Bag5 was cloned by screening a cerebellar library combined with RT-PCR and 5′-RNA Ligase Mediated RACE from rat brain RNA (RLM-RACE, Ambion) (see FIG. 1) (GenBank® Accession Numbers rat is AY366364; human is NM_{—}004873 and mouse is XM_{—}127149).

Human Bag5 and Bag1 were cloned by RT-PCR from SH-SY5Y cells (ATCC) and were transferred into pDONR201 (Gateway, Invitrogen). The 5′ end of human Bag5 was cloned by RLM-RACE using human brain derived cDNA (Ambion). Sequencing was performed in both directions using multiple overlapping primers (ACGT, Toronto, ON). Discrepancies between sequences were resolved by analysis of chromatogram data. BAG5(DARA) was prepared with the QuikChangeXL kit (Stratagene). Human parkin and Hsp70 were transferred into pDONR201 by PCR from human brain cDNA (Ambion) and pMSHsp70, respectively. Human synphilin was subcloned into a C-terminus FLAG expression vector (Sigma). The GFPu proteasome reporter construct was generated as described by Bence et al., 2001, which is incorporated by reference. Control vectors included pEGFP-C1 (Clontech), pDsRed-C1 (Clontech) and pSV-beta galactosidase (Promega). Expression vectors were generated by transfer to a Destination vector (LR clonase, Invitrogen). Destination vectors (all N-terminal fusion): pDEST15 (GST fusion, prokaryotic), pDEST17 (hexa-histidine fusion, prokaryotic), pDEST26 or pDEST31 (hexa-histidine fusion), pDEST-EGFP (N-terminal GFP fusion), and pDEST-HA, -myc, or -FLAG. pDEST-EGFP, -HA, -myc, and -FLAG were generated using the Gateway Conversion System (Invitrogen) and pEGFP-C1 (Clontech), pCMV-HA (Clontech), pCMV-Myc (Clontech) and pFLAG-CMV2 (Sigma), respectively. For each construct the integrity of the epitope tag and cDNA was verified by sequencing.

### EXAMPLE 2

### Northern Blots

mRNA Northern blots (Origene) were probed with rat or human Bag5 cDNA or β-actin (Ambion) labeled with [³²P]ATP (NEN) using the StripEZ probe NorthernMax-Gly kits (Ambion).

### EXAMPLE 3

### In situ Hybridization (ISH), Immunohistochemistry (IHC) and Immunocytochemistry (ICC)

IHC was performed as we have previously described by Crocker et al., 2001, which is incorporated by reference. Rat BAG5 probes for ISH were labeled using [³³P]UTP (NEN) or digoxygenin (DIG) (Roche) via in vitro transcription with SP6 or T7 RNA polymerase (Promega). ICC was performed using conditions described by Junn et al. (2002) and Chung et al. (2001) and cells were analyzed using confocal microscopy (LSM-510 Meta, Zeiss).

### EXAMPLE 4

### Antibodies

Tyrosine hydroxylase (Immunostar), alpha-synuclein (BD Bioscience), Hsp70 (monoclonal, Stressgen), FLAG (M2, Cy3-M2 and polyclonal, Sigma), His-tag (Amersham), HA-tag (Roche), ubiquitin (Chemicon), parkin (Cell Signal), GAPDH (Chemicon), Beta-galactosidase (Promega) and GFP (polyclonal, Molecular Probes). BAG5 polyclonal antibodies were generated by Exalpha Biologicals against KLH-conjugated BAG5(118-134) peptide (Dalton). Affinity purified antibody was generated using a sulfhydrl coupling column (Sulfolink Kit, Pierce).

### EXAMPLE 5

### Cell Culture

HEK293T, NIH 3T3 and SH-SY5Y cells were maintained in DMEM growth medium supplemented with 10% fetal bovine serum, 0.5 mg/mL amphotericin B, 100 U/mL penicillin, and 100 mg/mL streptomycin (Invitrogen) at 37° C. in 5% CO₂.

### EXAMPLE 6

### Fusion Proteins

All GST- and His-fusion proteins were expressed in E. coli and affinity purified using Glutathione Sepharose 4B beads and HiTrap Columns (Amersham), respectively. His-Ubc6AC (C-terminal truncated Ubc6) and His-Ubc7 (Imai et al., 2001) were affinity purified using Ni-NTA Agarose (Qiagen).

### EXAMPLE 7

### Pull-Down Assays (PDAs) and Immunoprecipitation (IPs)

Samples were harvested in lysis-buffer (50 mM Tris-HCl pH 7.4, 150 mM NaCl, 2 mM EDTA, 1% Triton X-100) supplemented with protease inhibitors (Roche). For complete solubilization of the pellet in synphilin ubiquitinylation experiments boiling lysis-buffer with 1% SDS was used. IPs were performed overnight followed by incubation with Protein G sepharose beads (Amersham). PDAs were performed with equimolar amounts of GST- and His-fusion proteins for 1 hr (4° C.). Beads were washed once with lysis-buffer and three times with PBS (pH 7.4).

### EXAMPLE 8

### Luciferase Refolding Assay

Refolding assays were performed as described by Nollen et al. (2001) using indicated vectors and normalized for transfection efficiency by β-galactosidase activity (pSV-β-Galactosidase Control Vector, Promega).

### EXAMPLE 9

### Parkin Ubiquitinylation Assays

Ubiquitinylation was carried out by incubating 2 μg GST or GST-Parkin, 3 μg His-BAG5, 3 μg His-Hsp70, with mammalian E1 ubiquitin-activating enzyme (Boston Biochem) (180 nM), the E2 ubiquitin conjugating enzymes Ubc6ΔC (21 g) and Ubc7 (2 μg) and 20 μM Ub (Sigma), in 50 mM Tris (pH7.4), 100 mM NaCl, 5 mM MgCl₂, 1 mM DTT, 0.5 μM Ubiquitin Aldehyde (Boston Biochem) and 4 mM ATP. The reaction was incubated at 37° C. for 45 minutes and resolved by SDS-PAGE. Synphilin ubiquitinylation assays were performed in HEK293T cells as described by others (Chung et al., 2004; Chung et al., 2001). Cells were co-transfected with indicated vectors, and HA-ubiquitin and treated with 20 μM of the proteasome inhibitor MG132 for 6 hours prior to harvesting.

### EXAMPLE 10

### GFPu Proteasome Reporter Assays

The GFPu reporter construct was co-transfected with the indicated vectors in HEK293T cells and lysed at 48 hours using gel loading buffer. GFPu accumulation was measured by fluorescence microscopy and WB quantitation relative to transfection controls (DsRed and beta-galactosidase).

### EXAMPLE 11

### Cell Death Analysis

Similar to others (Chung et al., 2004) we transfected SH-SY5Y cells with the indicated plasmids for 24 hours followed by treatment with lactacystin for 18 hours and cell death was quantified with Hoechst33342 (Molecular probes) and propidium iodide (Molecular probes).

### EXAMPLE 12

### Intrastriatal Adenovirus Injection, MFB Axotomy and the MPTP model of PD

Intrastriatal adenovirus injections were performed in rats and mice as the inventors have previously described (Crocker et al., 2001; Smith et al., 2003). Adenovirus was generated by subcloning FLAG-BAG5 and FLAG-BAG5(DARA) into AdTrack-CMV using the AdEasy system (Quantum). Ad.GFP was generated using empty AdTrack-CMV shuttle vector. MFB axotomy in rats and MPTP injections in mice and analysis was performed as previously described (Crocker et al., 2001; Smith et al., 2003).

### EXAMPLE 13

### shRNA-Mediated Knock-Down of BAG5

Primers targeting human BAG5 were designed using the web-based RNAi OligoRetriever and cloned using standard PCR-based protocols available at the world wide website of Cold Spring Harbor Laboratory, for example. For U6_shRNA_b5, the primer (SEQ.ID.NO.7) 5′ AAA AAA ATC AAG ATA ACT AAG AAT ATT CTG CGC AAC AAG CTT CTT GCG CAG AAT ATT CTC AGC TAT CTC GAC GGT GTT TCG TCC TTT CCA CAA 3′ was used. Both Cassettes were integrated into pDEST31 at position 5485 by PCR-mediated insertion using QuikChange (Stratagene) (Geiser et al., 2001). RT-PCR to confirm depression of endogenous BAG5 mRNA was performed with primers that bridge an intron: 5′GCC CCG CTC AGA CCT AGT 3′ (SEQ.ID.NO.24) and 5′ ACG TTC TGT CTC CTG TGC TG 3′ (SEQ.ID.NO.25).

### EXAMPLE 14

### Differential Display

At 24 hours post-medial forebrain bundle (MFB) axotomy, utilizing twenty-four arbitrary primers and three different oligo dT anchored primers, we detected twenty-six differentially expressed transcripts (DETs) by differential display, on comparison of the ipsilateral axotomized nigral region to the contralateral control nigral region. The DETs are designated Substantia nigra axotomy model 1 (SNam1) through SNam26 (See Table 1). SNam11, a DET induced at 24 hours post-MFB axotomy, was identified as BAG5.

TABLE 1
Differentially Expressed Transcripts in the Ventral Mesencephalic
Region (VMR) at Twenty-Four Hours Following MFB- Axotomy.

| | Fragment | | | GenBank ® |
|---|---|---|---|---|
| DET | Size | Δ | Sequence Homology | Accession No. |
| SNam 1 | 352 bpr | ↓ | Karyopherin alpha-3 | NM_008466.2 |
| | | | (importin alpha-4) | |
| SNam 2 | 153 bpr | ↑ | Striatal mRNA of | AK081290 |
| | | | unknown function. | |
| SNam 3 | 147 bpr | ↑ | Developmentally | U95001 |
| | | | Regulated | |
| | | | Cardiac Factor | |
| SNam 4 | 449 bpr | ↑ | Ubiquitin-specific | XM_343415 |
| | | | protease 3 (USP 3) | |
| SNam 5 | 101 bpr | ↑ | AP-50 assembly protein | M23674 |
| SNam 6 | 130 bpr | ↑ | protocadherin 2C mRNA | XM_341600 |
| SNam 7 | 411 bpr | ↓ | rab-28 ras-homologous | NM_053978 |
| | | | G-protein | |
| SNam 8 | 215 bpr | ↑ | No Match | n/a |
| SNam 9 | 351 bpr | ↑ | ribosomal protein S19 | XM_218456 |
| SNam 10 | 322 bpr | ↑ | Mss4 protein | X70496 |
| SNam 11 | 144 bpr | ↑ | bcl2-associated | BC055762 |
| | | | athanogene 5 (BAG5) | |
| SNam 12 | 176 bpr | ↓ | N-acetylglucosamine- | BC055328 |
| | | | 6-sulfatase | |
| SNam 13 | 402 bpr | ↑ | protein | NM_178586 |
| | | | phosphatase 2B | |
| SNam 14 | 114 bpr | ↑ | No Match | n/a |
| SNam 15 | 175 bpr | ↑ | DNAJ domain- | XM_214238 |
| | | | containing protein MCJ | |
| SNam 16 | 86 bpr | ↑ | Inconclusive match- | n/a |
| | | | Multiple Hits | |
| SNam 17 | 147 bpr | ↑ | Multiple Hits: CDK109, | n/a |
| | | | 103 and rRNA | |
| SNam 18 | 254 bpr | ↑ | Multiple Hits: CDK109, | n/a |
| | | | 103 and rRNA | |
| SNam 19 | 222 bpr | ↑ | No Match - some | n/a |
| | | | homology to small | |
| | | | inducible cytokine | |
| | | | subfamily A20 and | |
| | | | to CLCN4 | |
| SNam 20 | 432 bpr | ↑ | ATPase Na/K | BC078902 |
| | | | Transport Beta Sub | |
| SNam 21 | 305 bpr | ↓ | No Match | n/a |
| SNam 22 | 306 bpr | ↑ | ADP-ribosylation | NM_026011 |
| | | | factor-like 10C (Arl10c) | |
| SNam 23 | 158 bpr | ↑ | Hypothetical protein | XM_238385 |
| | | | XP_238385 | |
| SNam 24 | 573 bpr | ↑ | Protein tyrosine | AF508964 |
| | | | kinase non-catalytic | |
| | | | form (NTRK2) (TrkB) | |
| SNam 25 | 435 bpr | ↓ | DIX domain containing | BC039960 |
| | | | protein DIXdc-1 protein | |
| SNam 26 | 307 bpr | ↑ | SNAP25 interacting | BC063144 |
| | | | protein 30 | |

↑—increased intensity on differential display gel
↓—decreased intensity on differential display gel
DET—differentially expressed transcript
SNam—substantia nigra axotomy model
bpr—base pairs
Acc. No.—GenBank ® Accession Number
n/a—not applicable

### EXAMPLE 15

### BAG5 Expression is Induced in Dopaminergic Neurons Following Injury

To identify molecular mediators of dopaminergic neurodegeneration, differential display analysis was employed following the unilateral transection of the axons of dopaminergic SNpc neurons within their projection pathway, the medial forebrain bundle (MFB), in rat. The partial cDNA of a transcript that increased in the SNpc when compared to the contralateral untransected side at 24 hours post-axotomy was expanded by RT-PCR and RACE. The full-length cDNA encoded for a 447 amino acid protein with high identity to human BAG5 (90.4% identity, GenBank® accession number NM_{—}004873) and mouse BAG5 (bcl-2 associated athanogene 5) (93.7% identity, GenBank®® accession number XM_{—}127149) (FIG. 1A).

Using species-specific probes directed against the open reading frame of BAG5 two transcripts of BAG5, which were widely and differentially expressed in multiple tissue types including rat and human brain, were identified (See FIG. 1B). In situ hybridization (ISH) analysis in rat brain using an anti-sense cRNA probe directed against rat BAG5 revealed BAG5 expression in cortex, hippocampus, red nucleus and the SNpc (FIG. 1C, upper panel). A control sense cRNA probe did not reveal any structure-specific signal above background (FIG. 1C, lower panel). ISH with digoxygenin (DIG)-labeled anti-sense rat BAG5 probes combined with immunohistochemistry (1HC) for tyrosine hydroxylase (TH), a marker for dopaminergic neurons, demonstrated that BAG5 mRNA was expressed in the dopaminergic neurons of the SNpc (FIG. 1D). An increase in BAG5 expression was verified in these neurons at 24 hours following MFB axotomy both qualitatively (FIG. 1D, upper panel vs. middle panel) and quantitatively (FIG. 1E).

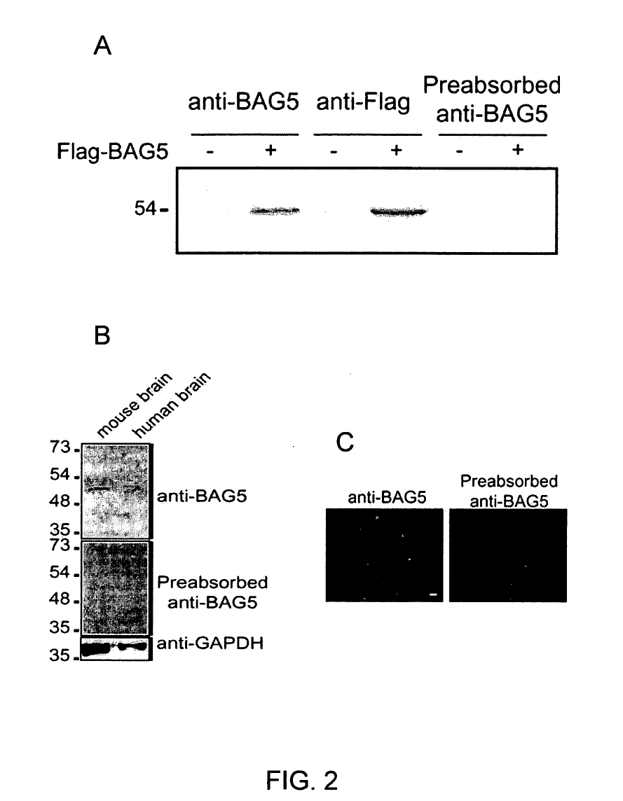

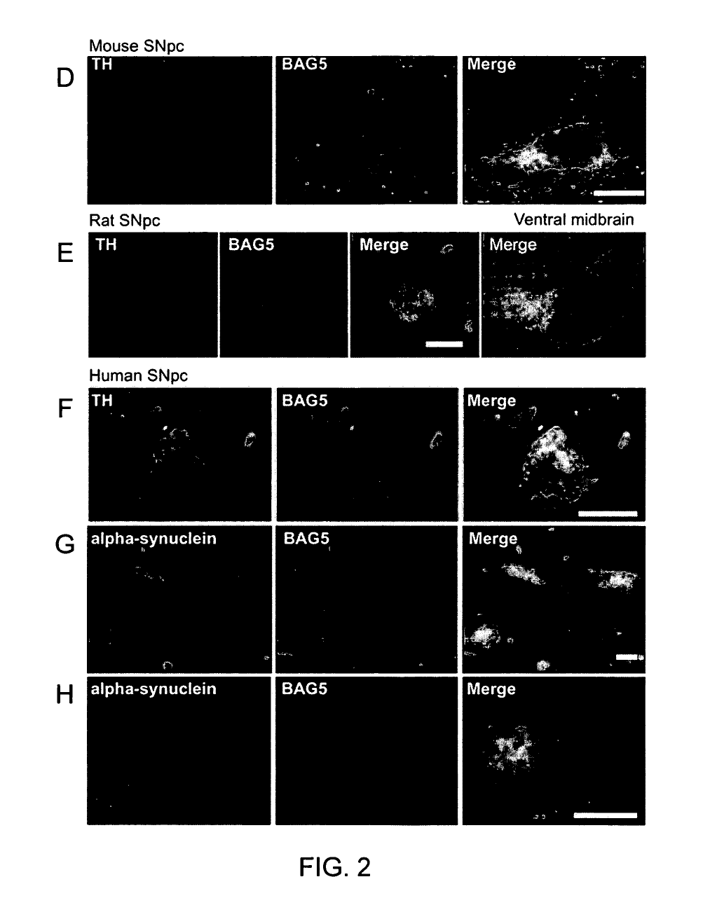

Polyclonal antibodies directed against BAG5 were used to examine BAG5 protein expression. The specificity of the BAG5 antibody was confirmed by Western blot analysis of cell lysates from the dopaminergic cell line SH-SY5Y transfected with N-terminus FLAG epitope-tagged BAG5 (FLAG-BAG5) (FIG. 2A). Both anti-FLAG and anti-BAG5 antibodies recognized a band with the predicted molecular weight. Pre-absorption of the anti-BAG5 antibody with its corresponding peptide immunogen competed out the signal. Probing lysates from mouse and human brain revealed a band of the expected molecular weight of approximately 51 kDa (FIG. 2B, upper panel); pre-incubation of the anti-BAG5 antibody with its corresponding peptide immunogen also competed out the signal as seen by Western blot (FIG. 2B, lower panel) and immunohistochemistry (FIG. 2C). The anti-BAG5 antibody also recognized rat BAG5, albeit with decreased sensitivity and occasionally a minor band of approximately 35 kDa in BAG5-transfected cell lysates and tissue lysates (data not illustrated). In mouse and rat SNpc, BAG5 immunoreactivity was found in all TH-immunopositive (TH+) dopaminergic neurons, as well as some TH-immunonegative (TH−) neurons (FIGS. 2D and 2E). Similarly, in human SNpc from a patient with Diffuse Lewy Body (DLB) disease, a neurodegenerative disorder similar to PD, BAG5 immunoreactivity was found in TH+ neurons (FIG. 2F). Alpha-synuclein is a major component of LBs (Spillantini et al., 1997) and it was found BAG5 immunoreactivity in alpha-synuclein immunoreactive neurons (FIG. 2G) and localized within all LBs (FIG. 2H). The finding that BAG5 expression was induced following dopaminergic neuron injury and was localized within dopaminergic neurons and LBs suggested that BAG5 may play a role in neurodegeneration.

### EXAMPLE 16

### BAG5 Interacts with and Inhibits the Molecular Chaperone Hsp70

Since other BAG-family proteins interact with the chaperone Hsp70 (Briknarova et al., 2001; Hohfeld and Jentsch, 1997; Sondermann et al., 2001; Takayama et al., 1997; Zeiner et al., 1997), a protein known to be present in LBs (Auluck et al., 2002; McLean et al., 2002) and to prevent both protein aggregation and neurodegeneration (Adachi et al., 2003; Auluck et al., 2002; Cummings et al., 2001; Sherman and Goldberg, 2001), in vitro GST pull-down assays were used to determine whether BAG5 also interacts directly with Hsp70.

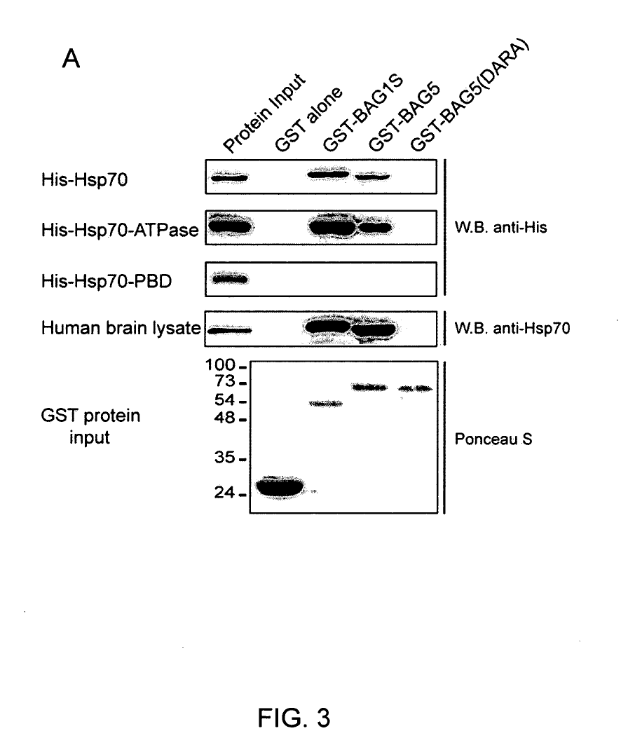

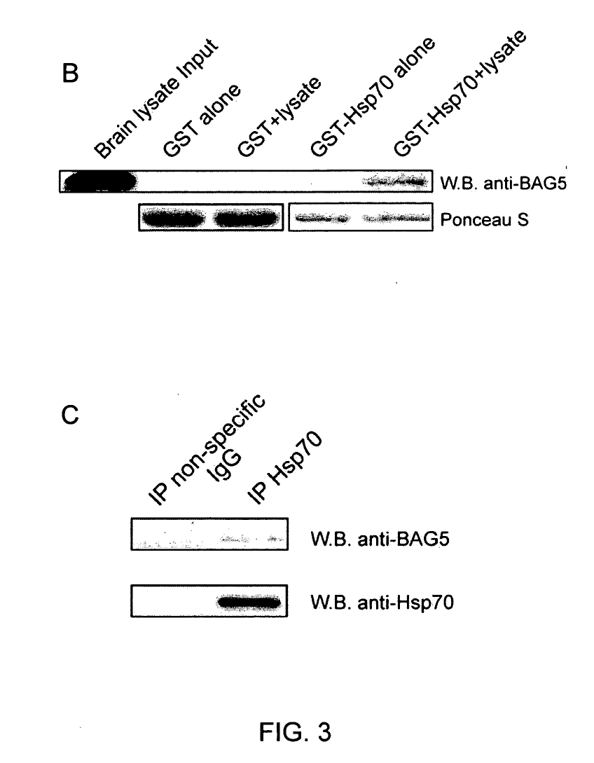

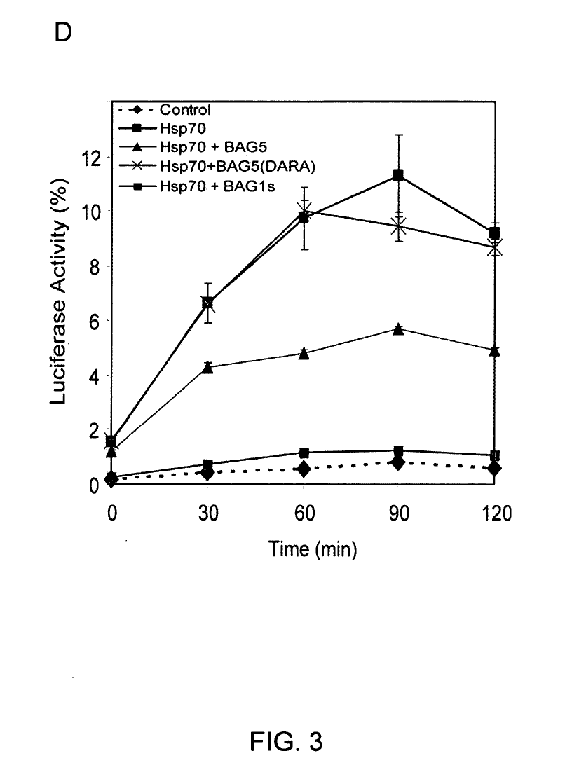

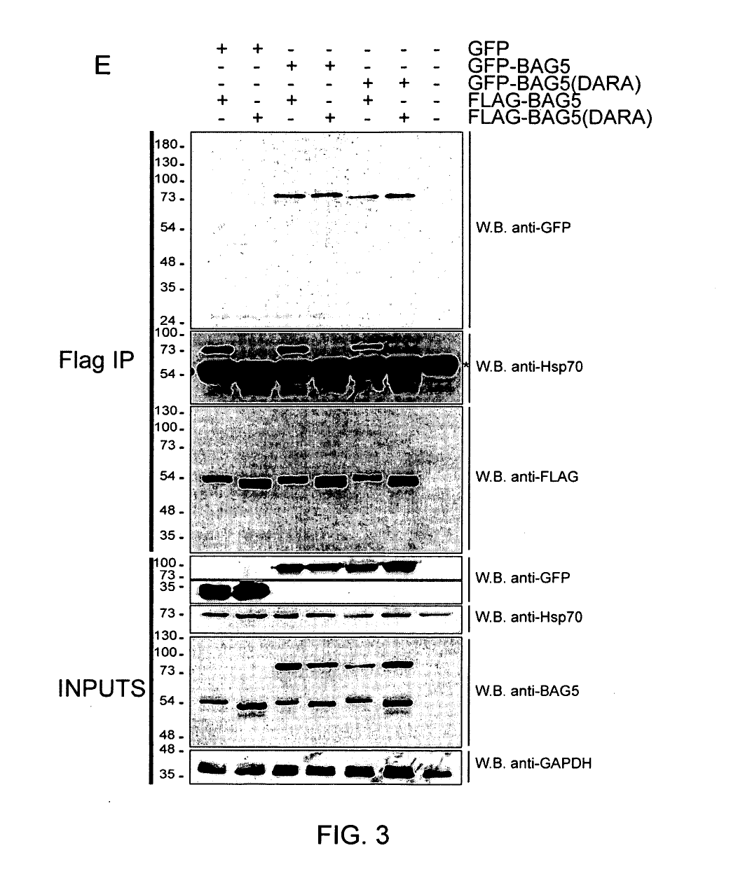

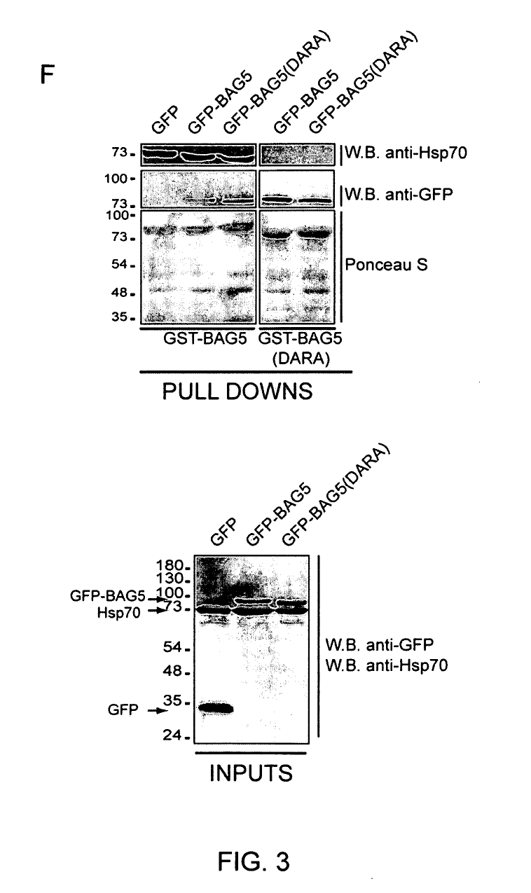

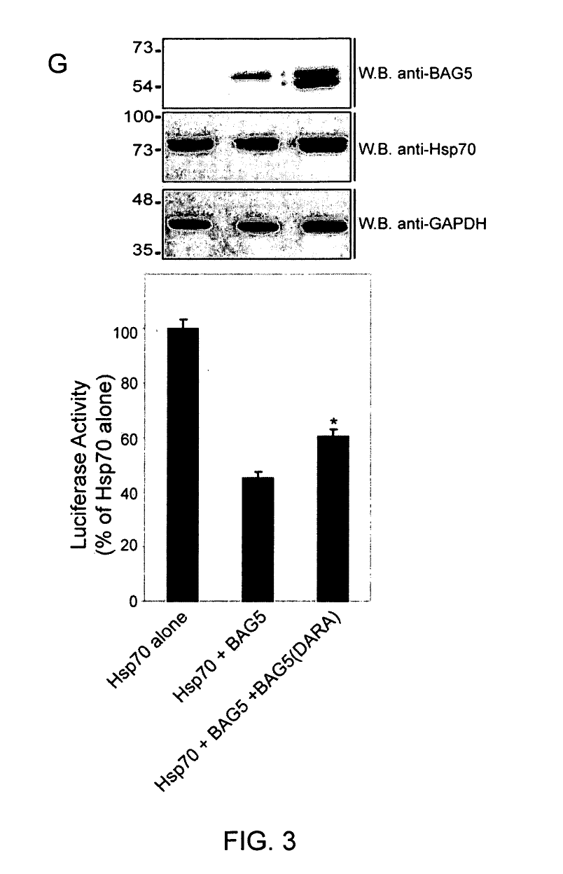

As shown in FIG. 3, GST-BAG5, like GST-BAG1, pulled down full-length Hsp70 in vitro whereas GST-alone did not (FIG. 3A). To identify the region of Hsp70 which mediates the interaction with BAG5, His-Hsp70 deletion constructs containing only the ATPase Domain (ATPase) or Protein Binding Domain (PBD) of Hsp70 were constructed. Both GST-BAG5 and GST-BAG1, but not GST-alone, pulled down the Hsp70 ATPase domain in vitro. In contrast, GST-BAG5, GST-BAG1 and GST-alone did not pull down the Hsp70 PBD (FIG. 3A). Therefore, the ATPase domain of Hsp70 was both necessary and sufficient for the interaction between BAG5 and Hsp70.

To further confirm the specificity of the interaction between BAG5 and Hsp70, BAG domains were aligned with the BAG domain of BAG1 to identify conserved amino acids. Based on our alignment and the crystal structure of the BAG domain of BAG1 bound to the Hsp70 ATPase domain (Sondermann et al., 2001) was substituted the conserved Aspartate (D) and Arginine (R) with Alanine (A) in all four predicted BAG domains by site-directed mutagenesis to generate the mutant BAG5(DARA) (FIG. 1A). Consistent with predictions from previous structural studies (Briknarova et al., 2001; Briknarova et al., 2002; Sondermann et al., 2001), substitution of these conserved residues in all four BAG domains abolished the interaction with full-length Hsp70 and the Hsp70 ATPase domain in pull-down assays using GST-BAG5(DARA). Furthermore, GST-BAG5 associated with endogenous Hsp70 in human brain lysate while GST alone or the mutant GST-BAG5(DARA) did not (FIG. 3A). In the converse experiment, the GST-Hsp70 fusion protein also associated with endogenous BAG5 (FIG. 3B). The physical association between BAG5 and Hsp70 in vivo was confirmed by performing co-immunoprecipitating experiments using human brain lysate (FIG. 3C). Thus, BAG5 interacts with Hsp70 in vitro and in vivo, and substitution of select residues within the BAG domains was sufficient to abolish this interaction.

The functional consequence of the interaction between Hsp70 and BAG5 were examined. Other BAG-family proteins, including BAG1, are co-chaperones of Hsp70 and can negatively regulate the ability of Hsp70 to refold misfolded proteins both in vitro (Hohfeld and Jentsch, 1997; Takayama et al., 1997; Zeiner et al., 1997) and in vivo (Nollen et al., 2001). Using a previously described system to investigate the effect of BAG1 on Hsp70 chaperone activity in cells (Nollen et al., 2000; Nollen et al., 2001), the effect of BAG5 on Hsp70 activity was examined. HEK293T cells with BAG1, BAG5 or the mutant BAG5(DARA) were transiently co-transfected with Hsp70 and a luciferase reporter. Luciferase activity was thermally inactivated and its reactivation by Hsp70 was measured at 30 minute intervals over 2 hours. By 2 hours post-inactivation, BAG5 and BAG1 significantly diminished Hsp70-mediated luciferase reactivation whereas the mutant BAG5(DARA) had no significant effect (FIG. 3D). These results showed that BAG5 negatively regulated the chaperone activity of Hsp70.

### EXAMPLE 17

### BAG5 Associates with Itself and with the Mutant BAG5(DARA)

Takayama et al. (1997) have suggested a minimal heterotetramer stoichiometry (2:2) for the BAG1-Hsp70 interaction and have confirmed dimer formation by BAG1 alone. Therefore, it was tested whether BAG5 may associate with itself by co-transfecting N-terminus GFP-tagged BAG5 (GFP-BAG5) with FLAG-BAG5 in HEK293T cells. Co-immunoprecipitation of GFP-BAG5 but not GFP alone with FLAG-BAG5 suggested that BAG5 can associate with itself (FIG. 3E, lane 3). Furthermore GFP-BAG5(DARA) also co-immunoprecipitated with FLAG-BAG5 suggesting that site-directed substitution within the BAG domains does not significantly alter the conformation of the mutant BAG5(DARA) (FIG. 3E, lane 5). Indeed, both GFP-BAG5 and GFP-BAG5(DARA) co-immunoprecipitated with FLAG-BAG5(DARA) (FIG. 3E, lane 4 and 6, respectively). It was also confirmed that Hsp70 co-immunoprecipitated with FLAG-BAG5 and not FLAG-BAG5(DARA) (FIG. 3E, lanes 1, 3 and 5 vs. 2, 4 and 6). Pull-down assays in cell lysates transfected with GFP-BAG5 and GFP-BAG5(DARA) demonstrated the interaction between BAG5 and itself, and BAG5 and the mutant BAG5(DARA). Similar to results from FIG. 3E, GST-BAG5 and GST-BAG5(DARA) interacted with both GFP-BAG5 and GFP-BAG5(DARA), whereas only GST-BAG5 interacted with Hsp70 (FIG. 3F). To determine if this association has a functional consequence, cells were co-transfected with BAG5 and BAG5(DARA) together in the luciferase refolding assay described in the previous examples, and it was found that the mutant BAG5(DARA) inhibited the effect of BAG5 on Hsp70-mediated refolding of the luciferase (FIG. 3G). Therefore complexes of BAG5 which contained BAG5(DARA) were unable to bind Hsp70 and inhibited BAG5 activity.

### EXAMPLE 18

### BAG5 Interacts Directly with Parkin

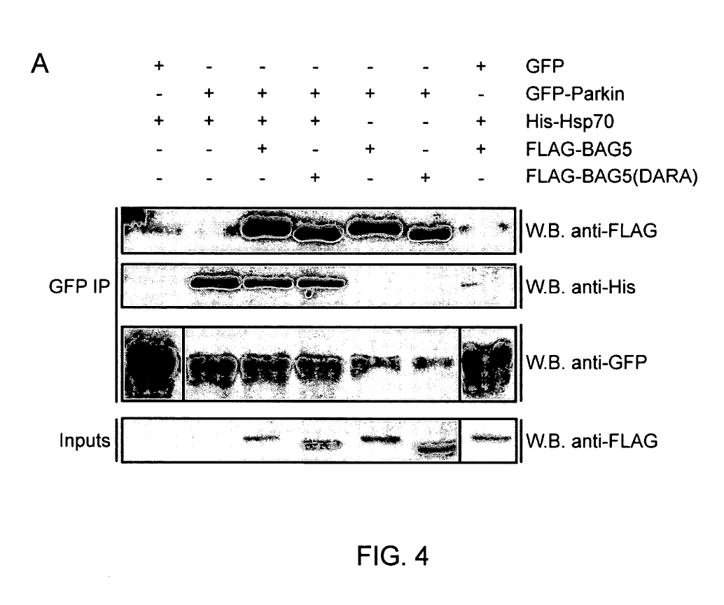

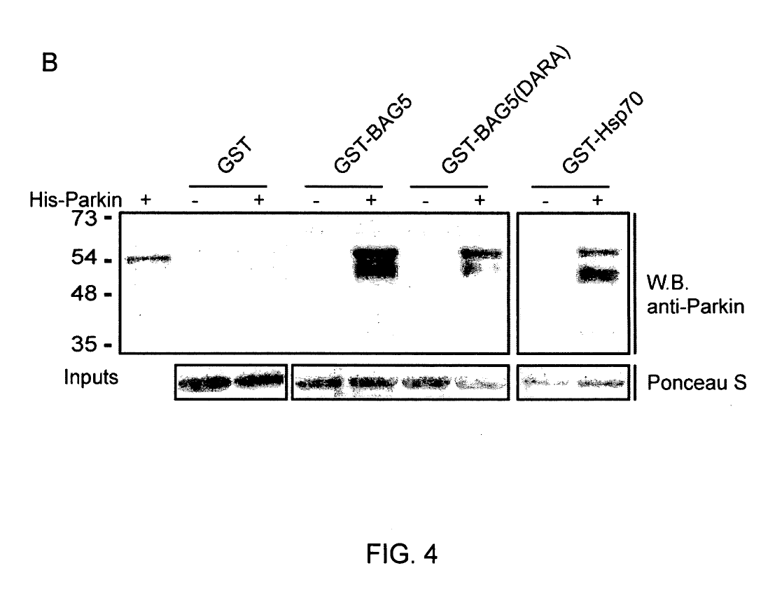

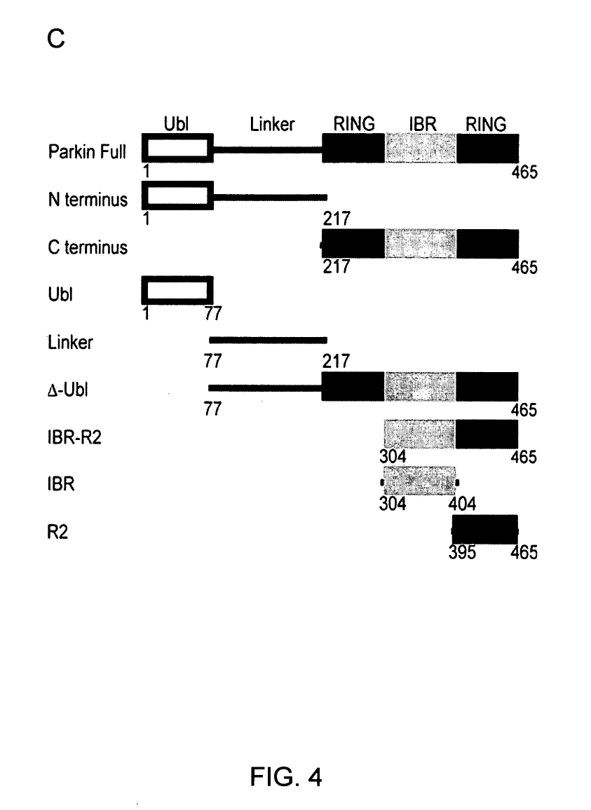

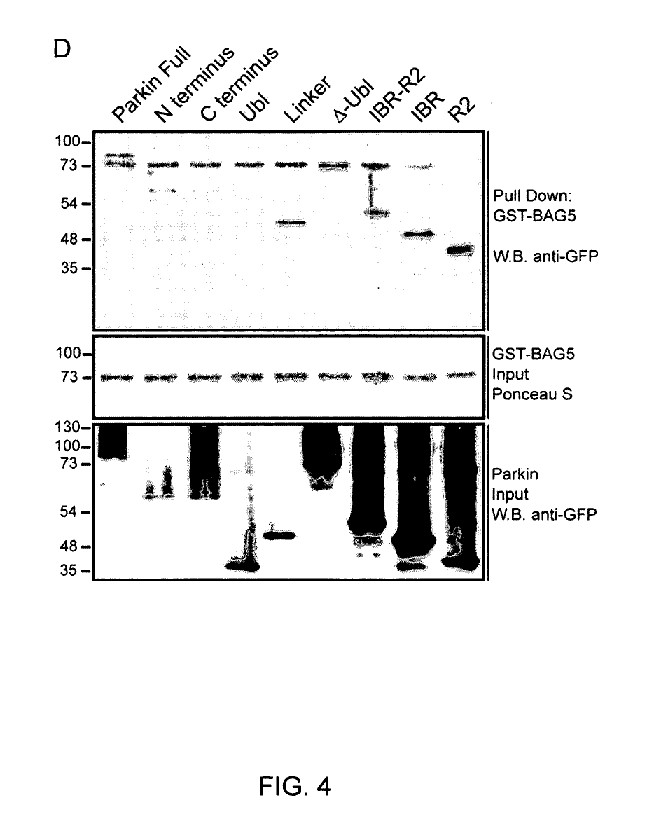

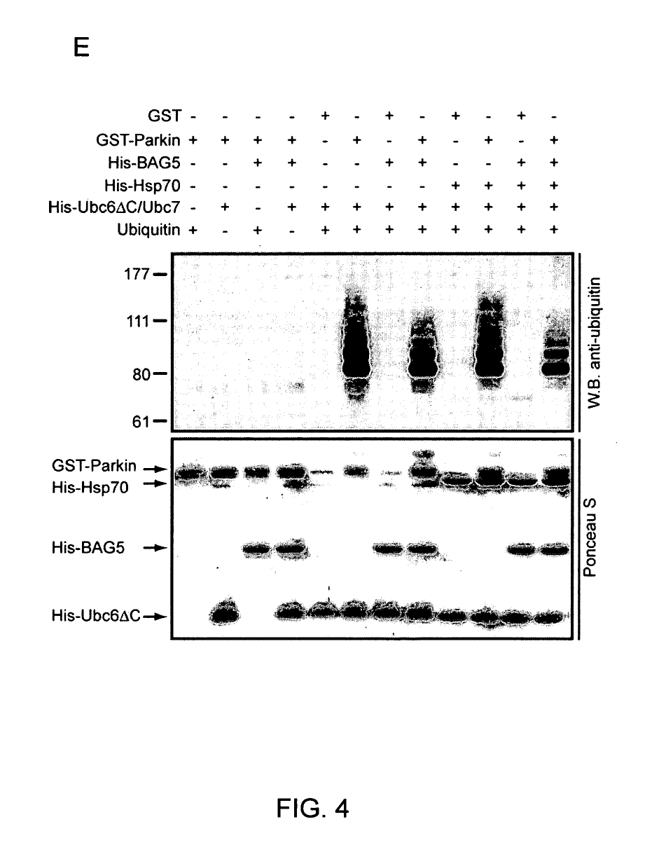

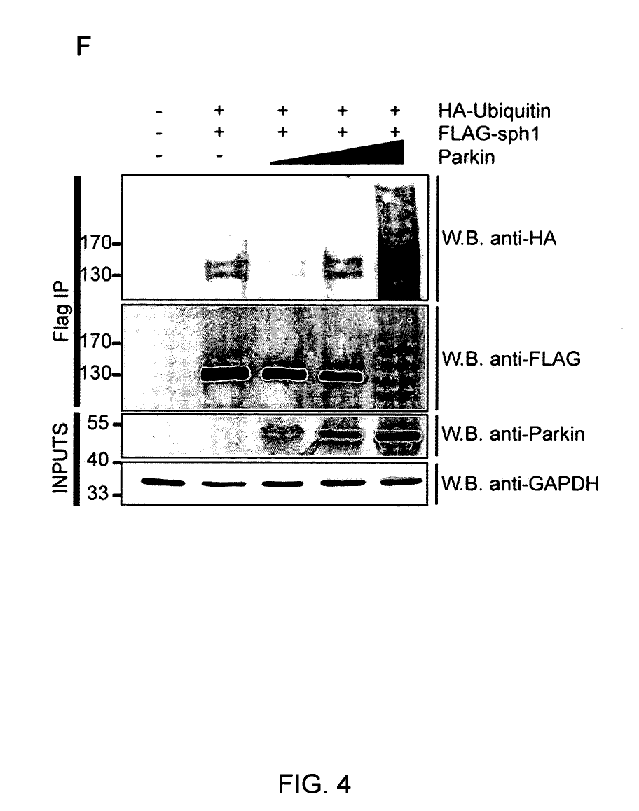

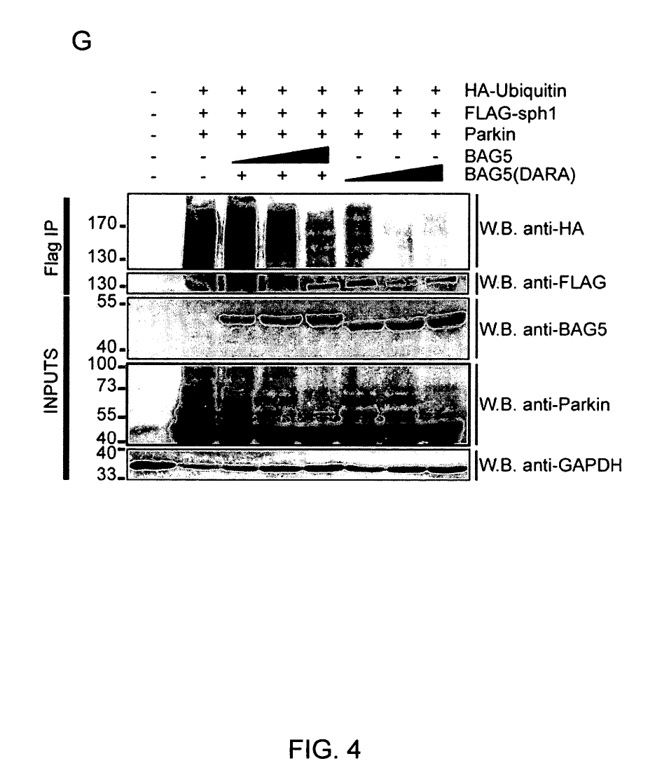

In brain, Hsp70 associates with parkin (Imai et al., 2002). Because of the importance of parkin in the pathogenesis of PD and its association with Hsp70, it was tested whether BAG5 associates in a complex with Hsp70 and parkin. Thus, HeK293T cells were co-transfected with GFP-parkin with FLAG-BAG5 or FLAG-BAG5(DARA) and His-Hsp70. Immunoprecipitation of GFP-parkin resulted in the co-immunoprecipitation of both Hsp70 and BAG5 or BAG5(DARA) (FIG. 4A). Furthermore, immunoprecipitation of GFP-parkin resulted in the co-immunoprecipitation of BAG5 or BAG5(DARA) in the absence of overexpressed Hsp70.

To further examine the nature of the association between parkin, Hsp70 and BAG5, in vitro pull-down experiments using GST-Hsp70, GST-BAG5, or GST-BAG5(DARA) with recombinant N-terminus His-tagged parkin were used. GST-Hsp70, GST-BAG5 and GST-BAG5(DARA) all interacted directly with His-parkin whereas GST-alone did not (FIG. 4B). Thus, BAG5 interacted directly with parkin in the absence of Hsp70.

It was sought to further dissect the interaction between BAG5 and parkin using a series of N-terminal GFP-tagged parkin deletion constructs (FIG. 4C). The parkin deletion constructs were individually transfected in HEK293T cells and pull-down assays were performed using GST-BAG5. BAG5 associated with both the N-terminus and C-terminus deletion constructs of parkin. Within the N-terminus of parkin, the BAG5 interaction was further mapped exclusively to the linker region of parkin (FIG. 4D). In agreement with previous results (Imai et al., 2002; Tsai et al., 2003), Hsp70 binding was limited to the C-terminus of parkin (data not illustrated). Taken together with the findings from the co-immunoprecipitation experiments and in vitro pull-down assays described above, these results established that BAG5, Hsp70 and parkin may associate in a complex. Furthermore, BAG5 interacted directly with parkin in the absence of Hsp70.

### EXAMPLE 19

### BAG5 Inhibits Parkin E3 Activity

Parkin, like other E3s, can undergo auto-ubiquitinylation (Chung et al., 2004; Staropoli et al., 2003; Zhang et al., 2000) and parkin E3 activity may be modulated by Hsp70 (Imai et al., 2002; Tsai et al., 2003). To investigate the potential role of BAG5 on parkin E3 activity, auto-ubiquitinylation of parkin in the presence or absence of BAG5 and/or Hsp70 in vitro was examined. GST-parkin, but not GST-alone, underwent in vitro auto-ubiquitinylation in the presence of E1 and the E2s Ubc6AC and Ubc7 (Imai et al., 2001) as detected by anti-ubiquitin antibodies (FIG. 4E). The removal of either ubiquitin or the E2s Ubc6AC and Ubc7 prevented auto-ubiquitinylation of parkin. The addition of His-Hsp70 alone did not affect auto-ubiquitinylation of parkin, whereas the addition of His-BAG5 alone significantly inhibited auto-ubiquitinylation. Furthermore, the addition of Hsp70 was unable to rescue the BAG5-mediated inhibition of auto-ubiquitinylation of parkin (FIG. 4E). Parkin-ubiquitinylation was confirmed by re-probing the blot with an anti-parkin antibody. The integrity and equivalent loading of all fusion proteins was verified by Ponceau S staining (FIG. 4E). Taken together, these data demonstrated that BAG5 directly inhibited parkin-mediated auto-ubiquitinylation independently of Hsp70.

Parkin has been shown to ubiquitinylate several substrates including the alpha-synuclein interacting protein synphilin (Chung et al., 2004; Chung et al., 2001). It was also observed an enhancement in the ubiquitinylation of C-terminus FLAG-tagged synphilin (FLAG-sph1) when co-transfected with parkin in HEK293T cells treated with the proteasome inhibitor MG132 (FIG. 4F). A BAG5 dose-dependent decrease in the amount of immunoprecipitated ubiquitinylated synphilin was observed (FIG. 4G). Similarly, the mutant BAG5(DARA), which retains the ability to directly interact with parkin, was tested and found that it also significantly inhibited parkin-mediated ubiquitinylation of synphilin (FIG. 4G). Therefore, BAG5 was a putative inhibitor of parkin E3 activity.

### EXAMPLE 20

### BAG5 Promotes Parkin Sequestration through the Inhibition of Hsp70

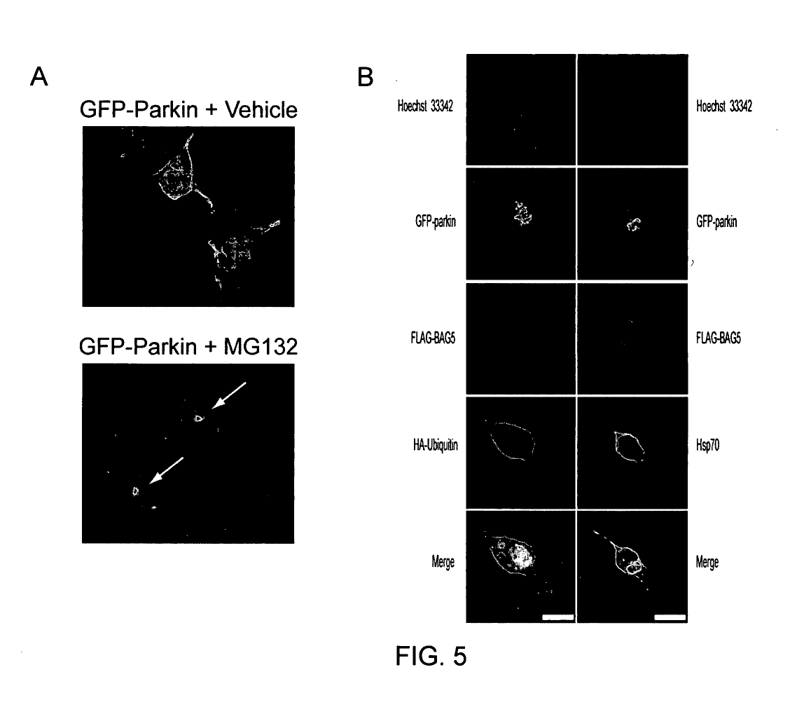

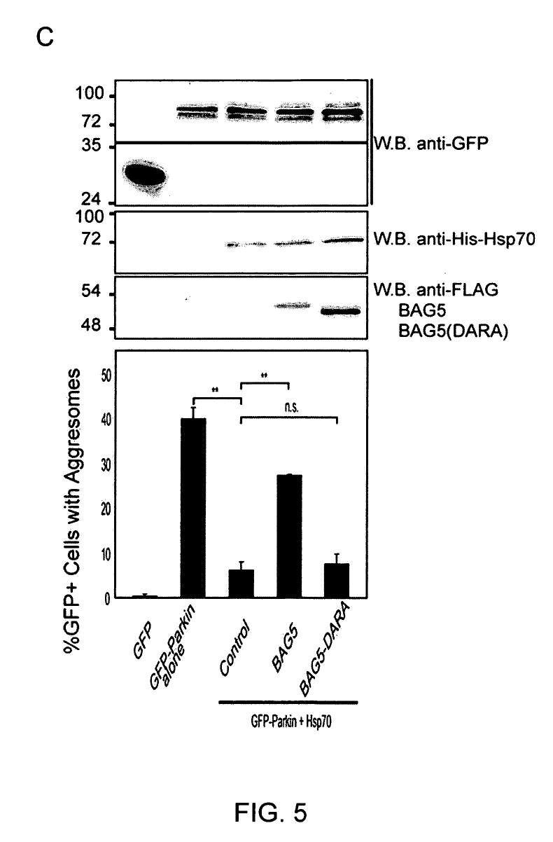

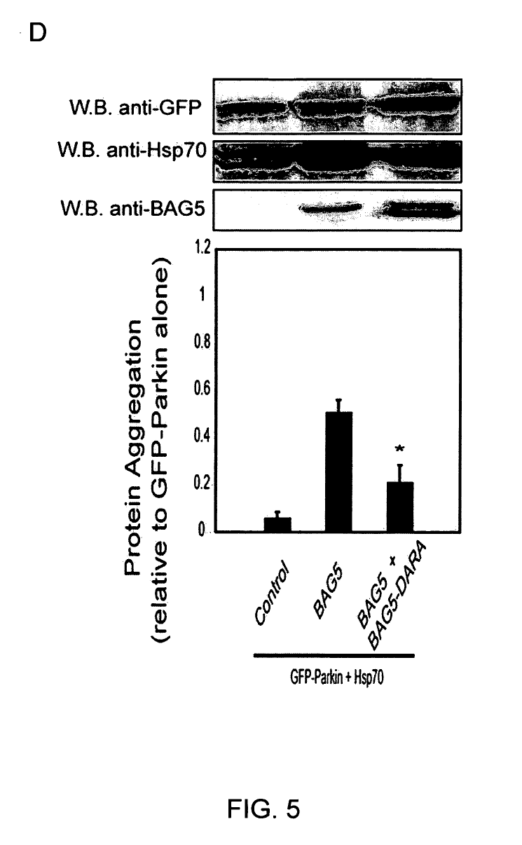

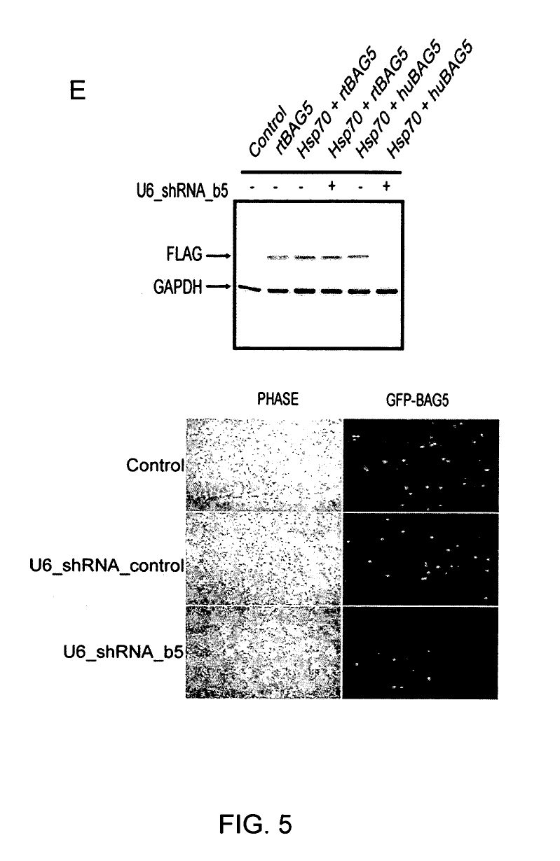

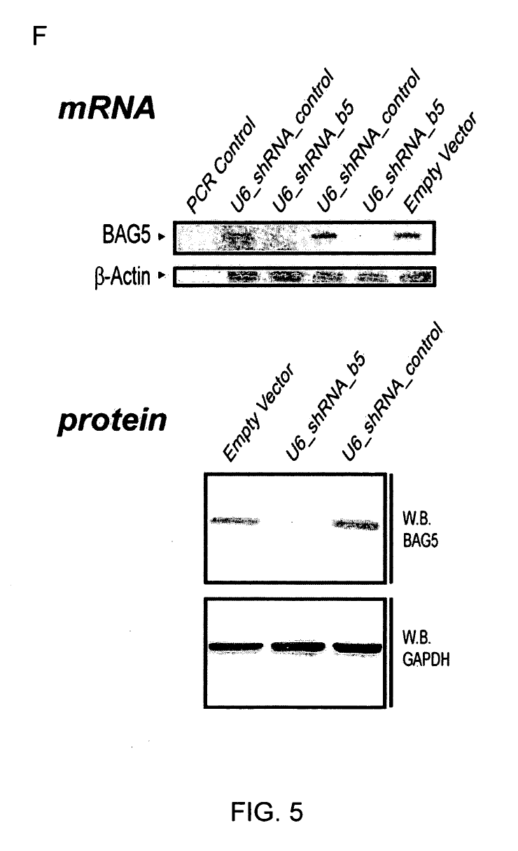

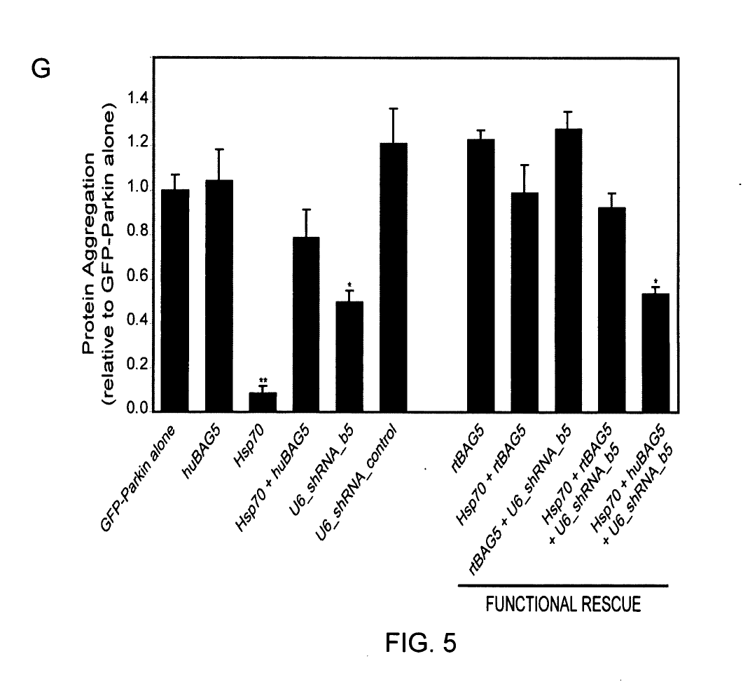

Proteasomal inhibition mediates the sequestration of parkin within perinuclear protein aggregates similar to the LBs of PD (Ardley et al., 2003; Junn et al., 2002; Muqit et al., 2003; Tsai et al., 2003; Winklhofer et al., 2003). In experiments similar to Junn et al. (2002) the inventors transfected HEK293T cells with GFP-parkin and treated the cells with MG132 to initiate the formation of protein aggregates (FIG. 5A). It was confirmed that the perinuclear parkin protein aggregates were immunoreactive for ubiquitin, and Hsp70 immunoreactivity was localized mainly at the periphery of the aggregate (FIG. 5B) as previously described (Junn et al., 2002). BAG5 immunoreactivity was also co-localized within these LB-like aggregates (FIG. 5B) as was alpha-synuclein and synphilin (data not illustrated). Furthermore, in agreement with previous studies (Ardley et al., 2003; Junn et al., 2002), 40±3% of GFP-parkin+ cells contained aggregates after MG132 treatment. To verify that the sequestration of parkin within protein aggregates was specific to parkin, the inventors transfected cells with GFP alone and found that less than 1% of cells contained any detectable inclusions (FIG. 5C).

The solubility of parkin and its mutants within a cell can be enhanced by the co-expression of the chaperones Hsp70 and Hsp40 with parkin (Winklhofer et al., 2003). Thus, it was examined whether Hsp70 and BAG5 had an effect on the formation of parkin-containing perinuclear aggregates. The co-transfection of BAG5 or BAG5(DARA) alone with GFP-parkin did not influence the formation of parkin-containing protein aggregates. Co-transfection of Hsp70 with GFP-parkin prior to proteasome inhibition significantly reduced the sequestration of parkin within aggregates to only 6±2% of GFP-parkin+ cells (p<0.001, Student t-test). However, BAG5 significantly inhibited the ability of Hsp70 to prevent aggregation, whereas co-transfection of the mutant BAG5(DARA) did not (FIG. 5C). Given that the mutant BAG5(DARA) inhibited the effect of BAG5 on Hsp70 (FIG. 3G) it was tested whether the co-expression of BAG5(DARA) could also mitigate BAG5 inhibition of Hsp70-mediated suppression of parkin sequestration. A significant decrease in the number of GFP-parkin containing protein aggregates was found when BAG5 was co-transfected with BAG5(DARA) (FIG. 5D). The expression levels of GFP-parkin were similar in all conditions tested. Taken together, these data suggested that BAG5, through its inhibition of Hsp70, promoted parkin sequestration and enhanced the formation of LB-like protein aggregates.

### EXAMPLE 21

### shRNA-Mediated Knock-Down of BAG5 Inhibits the Sequestration of Parkin

To further validate the functional role of BAG5 as an enhancer of parkin sequestration in protein aggregates, short hairpin RNA (shRNA)-mediated “knock-down” (Paddison et al., 2002) of BAG5 was used to determine its effect on the sequestration of parkin. U6_shRNA_b5, a U6 promoter driven shRNA directed against human BAG5 was able to significantly depress the expression of transfected FLAG-tagged human BAG5 in HEK293T cells whereas a U6_shRNA_control vector targeting luciferase did not (FIG. 5E, upper panel). Also, co-transfection of U6_shRNA_b5 with N-terminal GFP-tagged BAG5 in HEK293T cells depressed the expression of GFP-BAG5 protein whereas U6_shRNA_control had no effect on GFP-BAG5 (FIG. 5E, lower panels). Next, it was determined if U6_shRNA_b5 could depress the endogenous huBAG5 in cells, and it was found that U6_shRNA_b5 significantly depressed the relative expression of both endogenous BAG5 mRNA and protein whereas U6_shRNA_control did not (FIG. 5F). Then it was examined the relative effect of shRNA-mediated BAG5 knock-down on the sequestration of parkin in protein aggregates. Co-transfection of U6_shRNA_b5 with GFP-parkin significantly inhibited the sequestration of parkin in aggregates to 50±5% relative to GFP-parkin alone whereas U6_shRNA_control had no significant effect (FIG. 5G).

To confirm that the observed knock-down effect was due to the specific depression of BAG5, functional rescue experiments were performed with rtBAG5, which was highly homologous to huBAG5 but resistant to U6_shRNA_b5-mediated knock-down (FIG. 5E). Since BAG5 promotes parkin sequestration through its inhibition of Hsp70 (FIG. 5C), it was first examined if rtBAG5 also mitigated Hsp70-mediated inhibition of parkin sequestration in protein aggregates. Indeed, co-expression of rtBAG5 with Hsp70 and parkin prevented Hsp70-mediated inhibition of parkin sequestration. Significantly, rtBAG5 was able to functionally rescue the loss of huBAG5 inhibition of Hsp70 function in the presence of U6_shRNA_b5 (FIG. 5G). Taken together, these data confirmed that BAG5 has a functional role in the sequestration of parkin in protein aggregates.

### EXAMPLE 22

### BAG5 Inhibits Parkin-Mediated Suppression of UPS Dysfunction and Cell Death

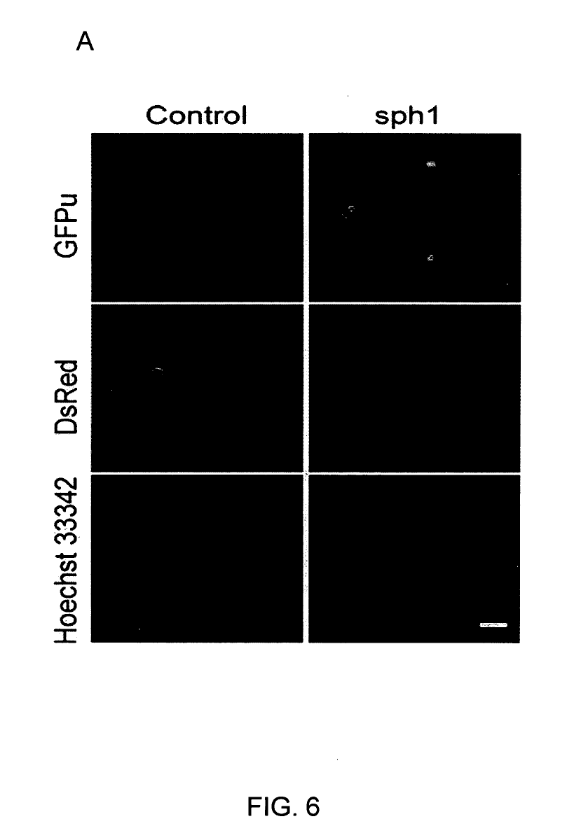

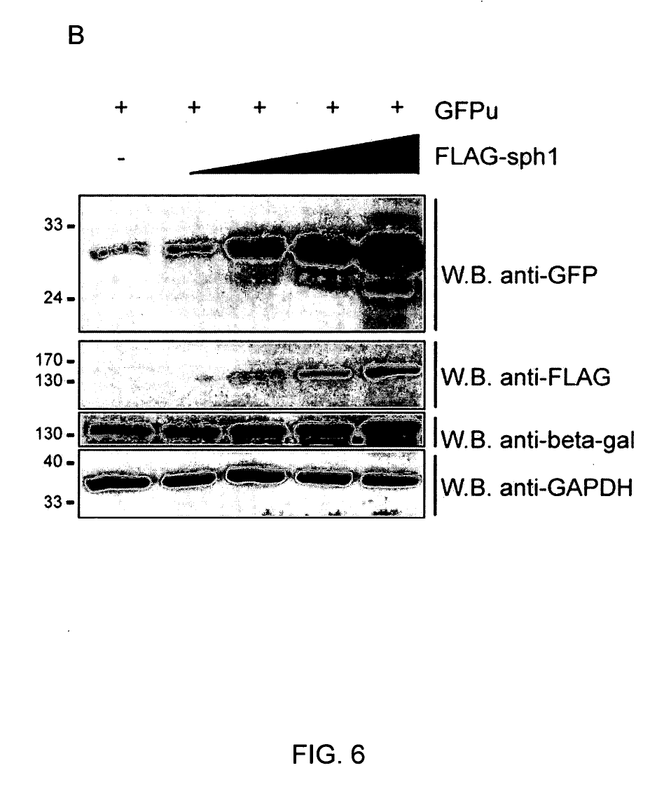

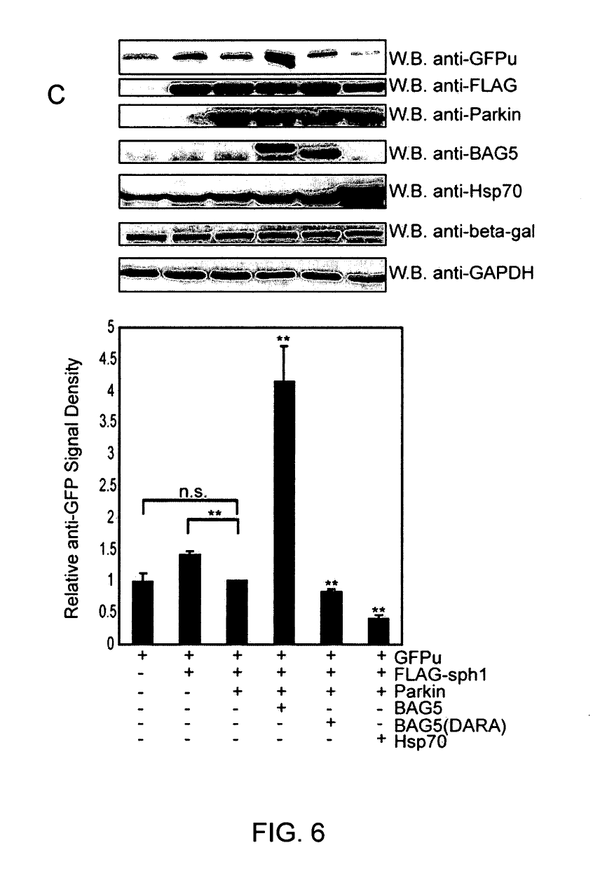

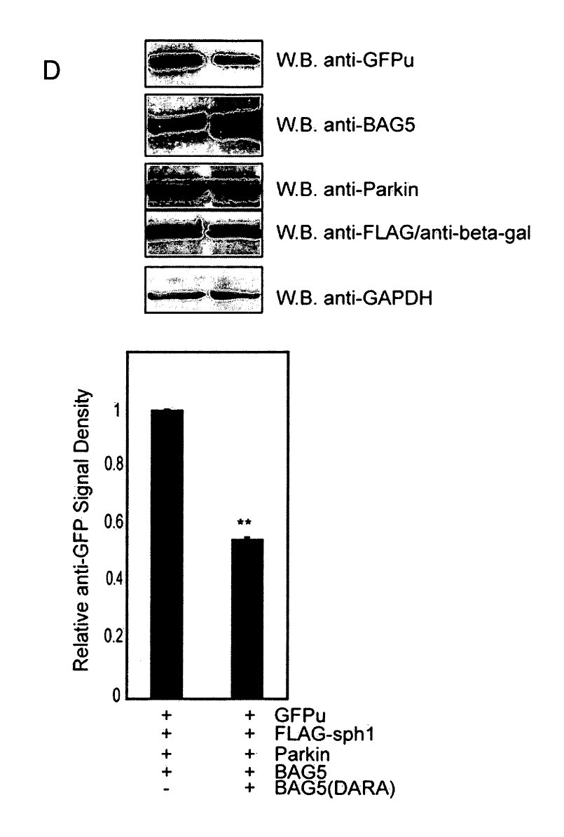

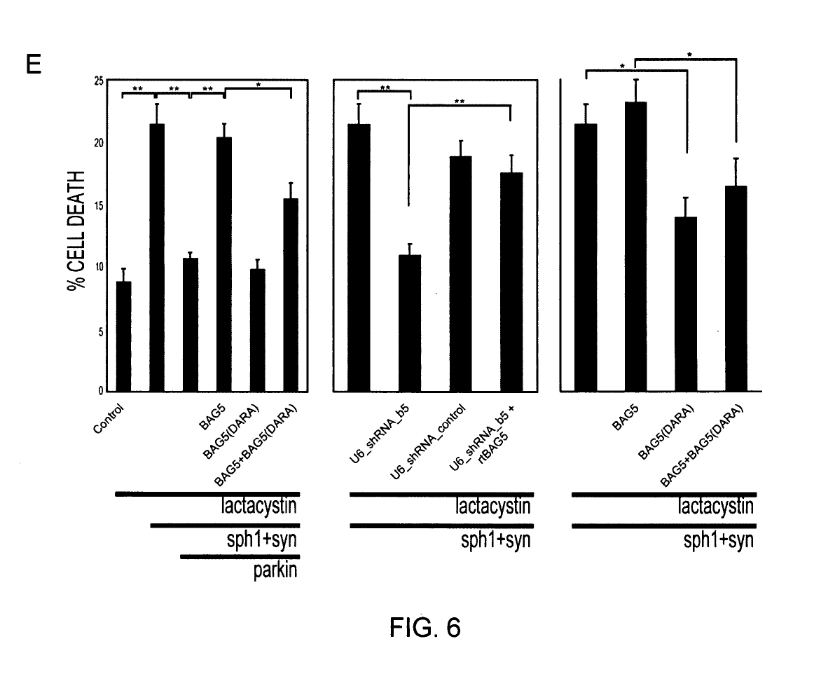

A GFPu reporter construct (Bence et al., 2001) allows for the measurement of proteasomal activity in living cells. GFPu has been shown to rapidly accumulate in settings of UPS dysfunction due to the expression of mutant proteins associated with neurodegenerative diseases (Bence et al., 2001; Petrucelli et al., 2002; Tsai et al., 2003). Upon co-transfection of synphilin with GFPu in HEK293T cells it was observed a significant accumulation and aggregation of GFPu relative to those cells co-transfected with empty vector (FIGS. 6A and 6B). To ensure similar transfection efficiency between conditions, DsRed and beta-galactosidase as transfection controls were used. Taken together these results suggested that the accumulation of synphilin alone was sufficient to induce proteasome inhibition as measured by the accumulation of GFPu.

The GFPu reporter is not a substrate of parkin and parkin has been shown to reduce proteasome impairment due to substrate accumulation as measured by the GFPu reporter system (Petrucelli et al., 2002; Tsai et al., 2003). Similarly, it was found that parkin mediated a significant decrease in GFPu accumulation due to synphilin expression (FIG. 6C). BAG5 reversed the inhibition of proteasome dysfunction by parkin and resulted in a significant increase in the accumulation of GFPu, whereas co-transfection of the mutant BAG5(DARA) or the chaperone Hsp70 resulted in a significant decrease in the accumulation of GFPu due to synphilin. Overexpression of BAG5 alone also resulted in a significant increase in the accumulation of GFPu due to synphilin expression (data not illustrated). Furthermore, co-transfection of the inhibitory mutant BAG5(DARA) with BAG5 mitigated proteasome dysfunction as measured by GFPu accumulation (FIG. 6D). Taken together these data suggested that BAG5 may contribute to proteasome impairment, in part, through the inhibition of parkin.

Parkin has also been shown to prevent cell death both in cell lines and in vivo (Imai et al., 2001; Imai et al., 2000; Kim et al., 2003; Oluwatosin-Chigbu et al., 2003; Petrucelli et al., 2002; Yang et al., 2003). Therefore, it was sought to determine whether the interaction between parkin and BAG5 has consequences on cell survival. As previously reported by others (Chung et al., 2004; Ihara et al., 2003) co-expression of synphilin and wild-type alpha-synuclein in SH-SY5Y cells in the presence of the irreversible proteasome inhibitor lactacystin results in increased cell death which can be suppressed by parkin (FIG. 6E, left panel). The protective effect of parkin was significantly reduced by co-expression of BAG5 (FIG. 6E, left panel), while the co-expression of BAG5 alone with synphilin and alpha-synuclein expression in the presence of lactacystin did not significantly enhance cell death (FIG. 6E, right panel). To further validate these results, the U6_shRNA_b5 construct was used to depress the expression of BAG5 and a significant decrease in cell death was found whereas expression of a U6_shRNA_control construct had no effect (FIG. 6E, middle panel). The specificity of this effect was demonstrated with functional rescue by co-expressing rat BAG5 with the U6_shRNA_b5 construct. Finally, the mutant BAG5(DARA) was found to suppress both the inhibitory effect of BAG5 on parkin (FIG. 6E, left panel) and when expressed alone (FIG. 6E, right panel).

### EXAMPLE 23

### BAG5 Enhances Dopaminergic Neurodegeneration In Vivo

The present invention has implicated BAG5 as a negative modulator of parkin and Hsp70, two molecules with known neuroprotective properties.

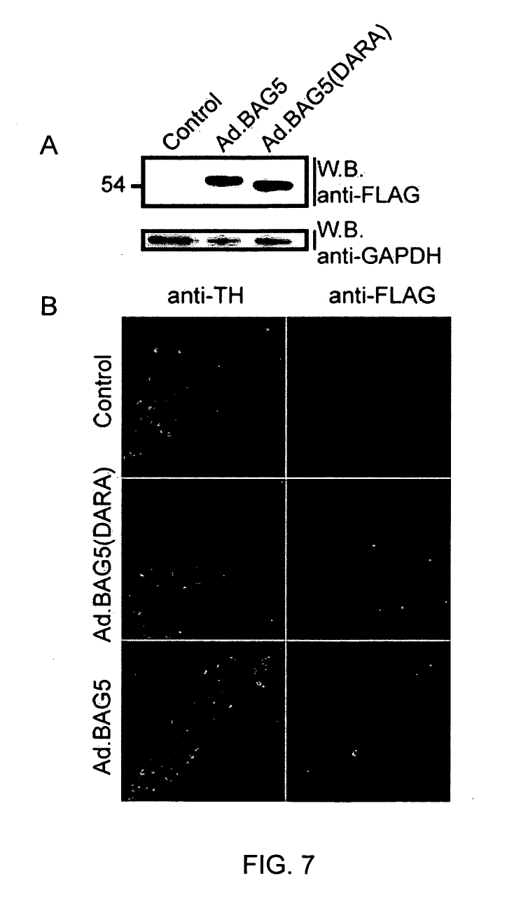

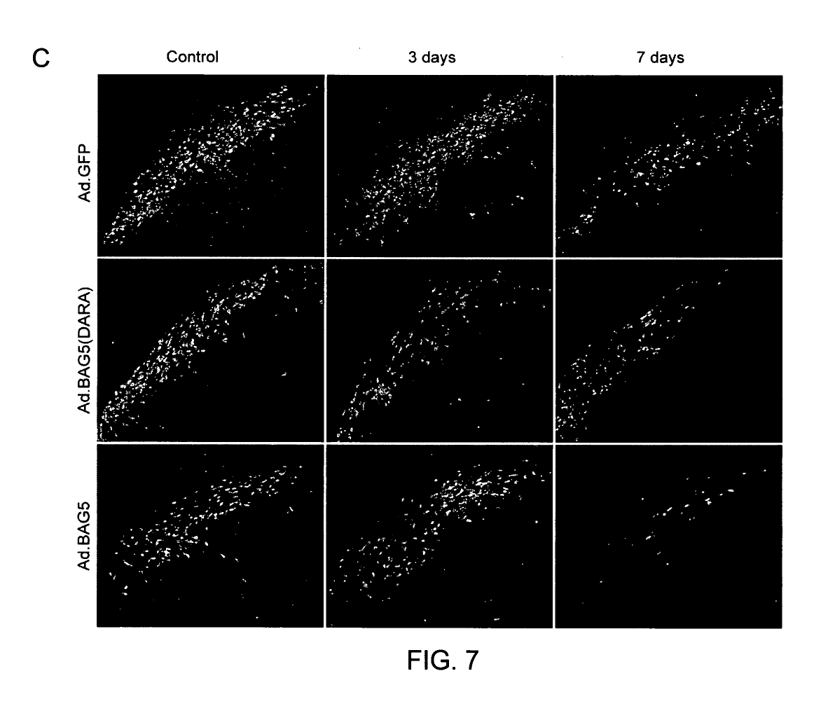

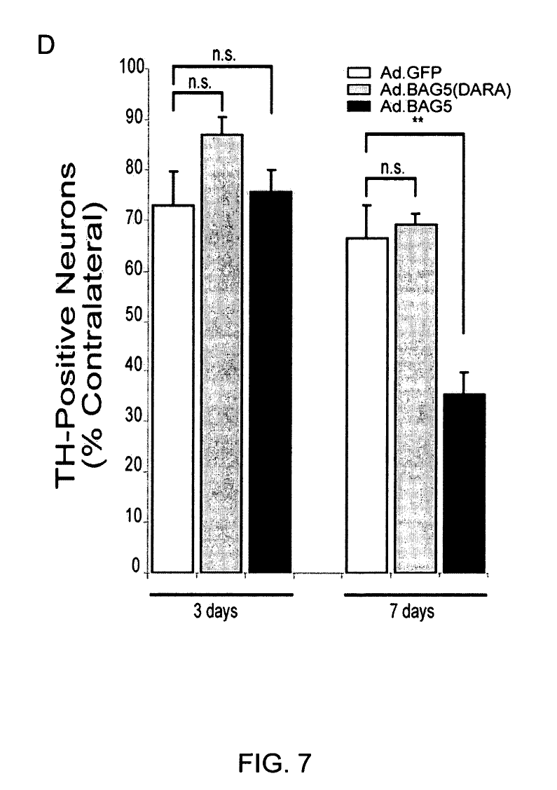

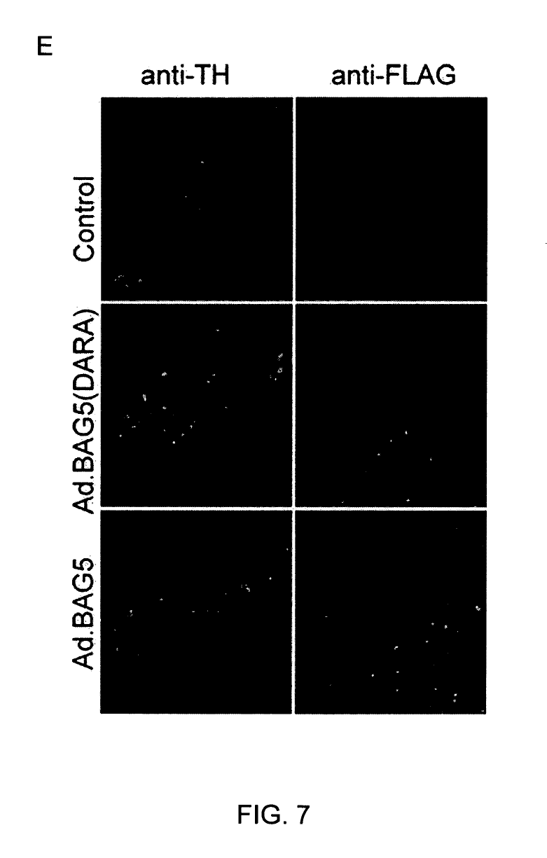

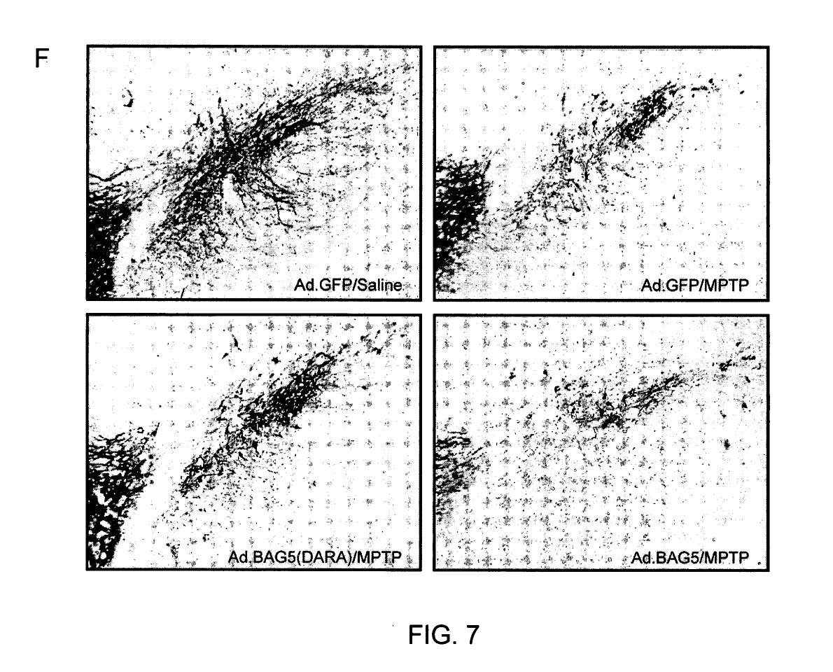

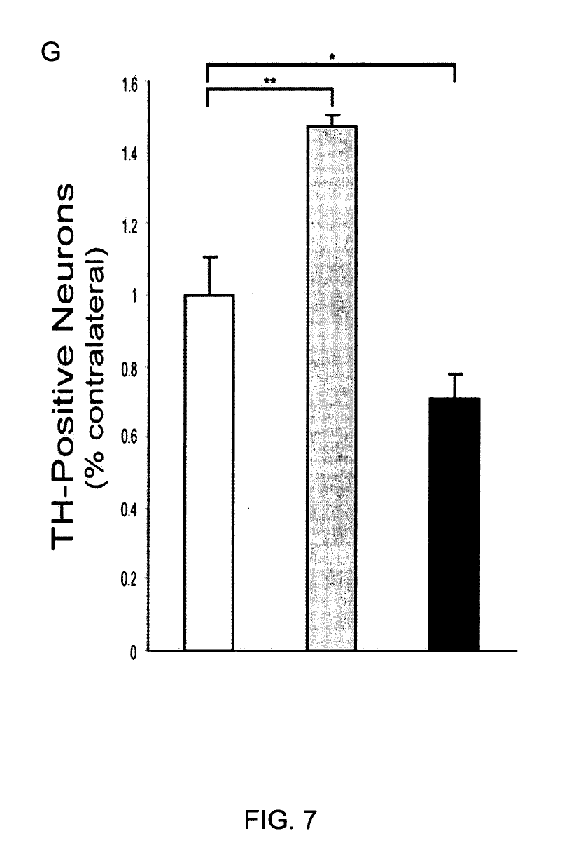

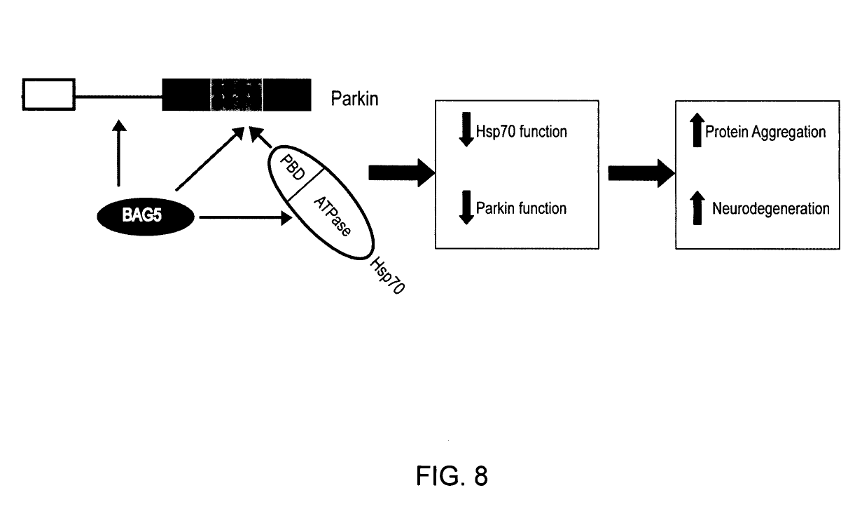

Briefly, a FLAG-BAG5 expressing adenovirus (Ad.BAG5), a FLAG-BAG5(DARA) expressing adenovirus (Ad.BAG5(DARA)) and a control adenovirus (Ad.GFP) were constructed. The expression of both FLAG-BAG5 and FLAG-BAG5(DARA) was verified by Western blot analysis of infected cell lysates using both anti-FLAG antibodies (FIG. 7A) and anti-BAG5 antibodies (data not illustrated). Targeted over-expression of adenovirus was achieved in the SNpc at the level of the medial terminal nucleus (MTN) in rat following the unilateral injection of virus in the ipsilateral striatum as we have described previously (Crocker et al., 2001; Smith et al., 2003). Viral expression in the SNpc was verified by immunohistochemistry with FLAG-BAG5 or FLAG-BAG5(DARA) immunoreactivity detected in TH+ dopaminergic neurons at 4 weeks post-injection (FIG. 7B).

To determine whether BAG5 enhances the loss of dopaminergic neurons following injury, MFB axotomy was performed 1 week after injection of Ad.GFP, Ad.BAG5 or Ad.BAG5(DARA) and examined the degree of dopaminergic neuron loss at 3 and 7 days post-MFB axotomy. At day 3 post-axotomy, quantitation of the TH+ neurons of the treated SNpc relative to the untreated contralateral side revealed no difference in neuronal survival between animals treated with Ad. BAG5, Ad.BAG5(DARA) or Ad.GFP (FIG. 7C). However, by day 7 post-axotomy, animals receiving Ad.BAG5 showed increased death of dopaminergic neurons with only 35±5% of neurons surviving relative to control vs. 66±7% (mean±s.e.m., p=0.001, Student t test) of neurons surviving relative to control in animals receiving Ad.GFP (FIGS. 7C and 7D). Loss of dopaminergic neurons was verified by cresyl-violet staining to rule out the possibility that TH protein expression was down-regulated post-axotomy.

Next, the effect of targeted expression of BAG5 and the inhibitory mutant BAG5(DARA) was examined in the SNpc in the well defined 1-methyl-4-phenyl-1,2,3,6-tetrahydropyridine (MPTP) model of PD (Dauer and Przedborski, 2003; Vila and Przedborski, 2003). Recently the loss of parkin function has also recently been implicated in neuronal death in this model (Chung et al., 2004; Yao et al., 2004). It was found that the targeted expression of BAG5 in the SNpc of mice by Ad.BAG5 (FIG. 7E) significantly enhanced the loss of dopaminergic neurons in the SNpc ipsilateral to the intrastriatal viral injection at 2 weeks following MPTP administration when compared to those animals injected with the control virus Ad.GFP (FIGS. 7F and G). Next, effect of mutant BAG5(DARA) (FIG. 7E) was examined, which was shown to bind to and mitigate the inhibitory effects of BAG5 on Hsp70 and parkin function (FIGS. 3E-G, 5D, 6D-E). It was determined that targeted expression of BAG5(DARA) resulted in a significant increase in dopaminergic neuronal survival (FIGS. 7F and G). Therefore, targeted expression of BAG5 in the SNpc enhanced dopaminergic neurodegeneration whereas targeted expression of the inhibitory mutant BAG5(DARA) in the SNpc suppressed dopaminergic neurodegeneration in an in vivo model of PD.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Adachi, H., et al., (2003). J Neurosci 23, 2203-2211.
Alberti, S., et al., (2002). J Biol Chem 277, 45920-45927.
Ardley, H. C., et al., (2003). Mol Biol Cell 22, 22.
Auluck, P. K., et al., (2002). Science 295, 865-868.
Batulan, Z., et al., (2003). J Neurosci 23, 5789-5798.
Bence, N. F., et al., (2001). Science 292, 1552-1555.
Brehmer, D., et al, (2001). Nat Struct Biol 8, 427-432.
Briknarova, K., et al, (2001). Nat Struct Biol 8, 349-352.
Briknarova, K., et al., (2002). J Biol Chem 277, 31172-31178.
Brive, L., et al., (2001). Biochem Biophys Res Commun 289, 1099-1105.
Chan, H. Y., et al., (2000). Hum Mol Genet 9, 2811-2820.
Chung, K. K., et al., (2004). Science 304, 1328-1331.
Chung, K. K., et al., (2001). Nat Med 7, 1144-1150.
Corti, O., et al., (2003). Hum Mol Genet 12, 1427-1437.
Crocker, S. J., et al., (2001). PNAS 98, 13385-13390.
Cummings, C. J., et al., (2001). Hum Mol Genet 10, 1511-1518.
Dauer, W., and Przedborski, S. (2003). Neuron 39, 889-909.
Demand, J., et al., (2001). Curr Biol 11, 1569-1577.
Doong, H., et al., (2003). J Biol Chem 278, 28490-28500.
Geiser, M., et al., (2001). Biotechniques 31, 88-90, 92.
Giasson, B. I., and Lee, V. M. (2003). Cell 114, 1-8.
Hershko, A., and Ciechanover, A. (1998). Annu Rev Biochem 67, 425-479.
Hohfeld, J., and Jentsch, S. (1997). Embo J 16, 6209-6216.
Huynh, D. P., et al., (2003). Hum Mol Genet 12, 12.
Ihara, M., et al., (2003). J Biol Chem 278, 24095-24102.
Imai, Y., et al., (2002). Mol Cell 10, 55-67.
Imai, Y., et al., (2001). Cell 105, 891-902.
Imai, Y., et al., (2000). J Biol Chem 275, 35661-35664.
Junn, E., et al., (2002). J Biol Chem 277, 47870-47877.
Kermer, P., et al., (2003). Brain Pathol 13, 495-506.
Kim, S. J., et al., (2003). J Biol Chem 12, 12.
Kitada, T., et al., (1998). Nature 392, 605-608.
Klucken, J., et al., (2004). J Biol Chem 279, 25497-25502.
Lang, A. E., and Lozano, A. M. (1998). N Engl J Med 339, 1044-1053.
Luders, J., et al., (2000). J Biol Chem 275, 4613-4617.
McLean, P. J., et al., (2002). J Neurochem 83, 846-854.
Muchowski, P. J., et al., (2000). Proc Natl Acad Sci USA 97, 7841-7846.
Muqit, M. M. K., et al., (2003). Hum Mol Genet 13, 117-135.
Nollen, E. A., et al., (2000). Mol Cell Biol 20, 1083-1088.
Nollen, E. A., et al., (2001). J Biol Chem 276, 4677-4682.
Nollen, E. A., and Morimoto, R. I. (2002). J Cell Sci 115, 2809-2816.
Oluwatosin-Chigbu, Y., et al., (2003). Biochem Biophys Res Commun 309, 679-684.
Paddison, P. J et al., (2002). Genes Dev 16, 948-958.
Petrucelli, L., et al., (2002). Neuron 36, 1007-1019.
Ren, Y., et al., (2003). JNeurosci 23, 3316-3324.
Schlossmacher, M. G., et al., (2002). Am J Pathol 160, 1655-1667.
Sherman, M. Y., and Goldberg, A. L. (2001). Neuron 29, 15-32.
Shimura, H., et al., (2000). Nat Genet 25, 302-305.
Shimura, H., et al., (2001). Science 293, 263-269.
Smith, P. D., et al., (2003). Proc Natl Acad Sci USA 100, 13650-13655.
Sondermann, H., et al., (2002). J Biol Chem 277, 33220-33227.
Sondermann, H., et al., (2001). Science 291, 1553-1557.
Song, J., et al., (2001). Nat Cell Biol 3, 276-282.
Spillantini, M. G., et al., (1997). Nature 388, 839-840.
Staropoli, J. F., et al., (2003). Neuron 37, 735-749.
Takayama, S., et al., (1997). Embo J 16, 4887-4896.
Takayama, S., and Reed, J. C. (2001). Nat Cell Biol 3, E237-241.
Takayama, S., et al., (2003). Oncogene 22, 9041-9047.
Takayama, S., et al., (1995). Cell 80, 279-284.
Tsai, Y. C., et al., (2003). J Biol Chem 278, 22044-22055.
Vila, M., and Przedborski, S. (2003). Nat Rev Neurosci 4, 365-375.
Vila, M., and Przedborski, S. (2004). Nat Med 10, S58-62.
Warrick, J. M., et al., (1999). Nat Genet 23, 425-428.
Winklhofer, et al., (2003). J Biol Chem 12, 12.
Yang, Y., et al., (2003). Neuron 37, 911-924.
Yao, D., et al., (2004). Proc Natl Acad Sci USA 101, 10810-10814.
Zeiner, M., et al., (1997). Embo J 16, 5483-5490.
Zhang, Y., et al., (2000). PNAS 97, 13354-13359.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of attenuating cell loss in an individual comprising the step of administering to at least one cell of the individual a bcl-2 associated athanogene (BAG) inhibitor, wherein the BAG inhibitor decreases expression and/or activity of BAG thereby attenuating cell loss.

2. The method of claim 1, wherein the cell loss comprises neuronal cell loss.

3. The method of claim 2, wherein neuronal loss is associated with neurodegeneration.

4. The method of claim 2, wherein the neuronal cell is selected from the group consisting of cholinergic, adrenergic, noradrenergic, dopaminergic, serotonergic, glutaminergic, GABAergic, and glycinergic.

5. The method of claim 4, wherein the neuronal cell is a dopaminergic cell.

6. The method of claim 1, wherein the inhibitor is an antibody.

7. The method of claim 1, wherein the BAG inhibitor is selected from the group consisting of BAG1 inhibitor, BAG2 inhibitor, BAG3 inhibitor, BAG4 inhibitor, and BAG5 inhibitor.

8. The method of claim 7, wherein the BAG inhibitor is BAG5 inhibitor.

9. The method of claim 8, wherein the inhibitor is a nucleic acid molecule.

10. The method of claim 9, wherein the nucleic acid molecule comprises a mutated BAG5 nucleic acid molecule.

11. The method of claim 10, wherein the mutated BAG5 nucleic acid molecule encodes a polypeptide having the sequence of SEQ. ID. NO. 8.

12. The method of claim 9, wherein the nucleic acid molecule is an antisense molecule.

13. The method of claim 12, wherein the antisense molecule is an siRNA molecule.

14. The method of claim 12, wherein the antisense molecule is an shRNA molecule.

15. The method of claim 14, wherein the shRNA molecule is encoded by the nucleic acid sequence of SEQ. ID. NO. 7.

16. The method of claim 9, wherein the nucleic acid sequence is comprised in an expression vector.

17. The method of claim 1, wherein the BAG inhibitor is prepared by the process of designing or selecting a candidate substance suspected of having the ability of decreasing BAG activity or BAG expression.

18. The method of claim 1, wherein the inhibitor increases HSP-70 chaperone activity in at least one cell of the individual.

19. The method of claim 1, wherein the inhibitor increases parkin E3 ubiquitin-ligase activity in at least one cell of the individual.

20. The method of claim 1, wherein the inhibitor decreases sequestration of parkin in at least one cell of the individual.

21. The method of claim 1, wherein the inhibitor decreases protein aggregation in at least one cell of the individual.

22. The method of claim 1, wherein the individual has a neurodegenerative disease.

23. The method of claim 1, wherein the individual is a human.

24. The method of claim 22, wherein the neurodegenerative disease is selected from the group consisting of Alzheimer's Disease, Parkinson's Disease, Huntington's Disease, amyotrophic lateral sclerosis (ALS), Guillain-Barre syndrome, multiple sclerosis, epilepsy, myasthenia gravis, chronic idiopathic demyelinating disease (CID), neuropathy, ataxia, dementia, chronic axonal neuropathy and stroke.

25. The method of claim 24, wherein the neurodegenerative disease is Parkinson's Disease.

26. The method of claim 25, wherein the Parkinson's Disease is sporadic Parkinson's Disease.

27. An isolated nucleic acid sequence that encodes a BAG5 shRNA molecule, wherein the sequence is SEQ. ID. NO. 7.

28. An isolated nucleic acid sequence that encodes a mutated BAG5 polypeptide having the sequence of SEQ. ID. NO. 8.

29. An expression vector comprising the nucleic acid sequence of claim 27.

30. An expression vector comprising the nucleic acid sequence of claim 28.

31. A method of manufacturing a BAG inhibitor comprising:
(a) providing a candidate substance suspected of decreasing BAG expression and/or activity;
(b) selecting the BAG inhibitor by assessing the ability of the candidate substance to decrease BAG expression and/or activity; and
(c) manufacturing the selected BAG inhibitor.

32. The method of claim 31, wherein the candidate substance is a protein, a nucleic acid molecule, an organo-pharmaceutical, or a combination thereof.

33. The method of claim 31, wherein the providing step is further defined as providing in a cell or a cell-free system a BAG polypeptide and the BAG polypeptide is contacted with the candidate substance.

34. The method of claim 31, wherein the candidate substance is a protein.

35. The method of claim 34, wherein the protein is an antibody that binds immunologically to BAG5.

36. The method of claim 31, wherein the providing step is further defined as providing a nucleic acid molecule that encodes the BAG polypeptide.

37. The method of claim 31, wherein the candidate substance is a nucleic acid molecule.

38. The method of claim 37, wherein the nucleic acid molecule is a mutated BAG nucleic acid molecule.

39. The method of claim 37, wherein the nucleic acid molecule is an antisense molecule.

40. The method of claim 37, wherein the nucleic acid molecule is an siRNA molecule.

41. The method of claim 37, wherein the nucleic acid molecule is an shRNA molecule.

42. The method of claim 31, further comprising the step of administering the BAG inhibitor to an individual in need thereof.

43. A pharmaceutical composition comprising an inhibitor manufactured according to claim 27 admixed with a pharmaceutical carrier.
